(19) United States
(12) Patent Application Publication
(10) Pub. No.: US 2024/0218022 A1
(43) Pub. Date: Jul. 4, 2024

(54) ANTI-VISTA MACROCYCLIC PEPTIDES AND COMPOSITIONS

(71) Applicant: BRISTOL-MYERS SQUIBB COMPANY, PRINCETON, NJ (US)

(72) Inventors: JENNIFER X. QIAO, ARLINGTON, MA (US); YUNHUI ZHANG, PRINCETON, NJ (US); KENNETH M. BOY, SOUTHBOROUGH, MA (US); MICHAEL A. POSS, LAWRENCEVILLE, NJ (US)

(73) Assignee: BRISTOL-MYERS SQUIBB COMPANY, PRINCETON, NJ (US)

(21) Appl. No.: 18/509,844

(22) Filed: Nov. 15, 2023

Related U.S. Application Data
(60) Provisional application No. 63/384,689, filed on Nov. 22, 2022.

(51) Int. Cl. C07K 7/64

(52) U.S. Cl. CPC C07K 7/64

(57) ABSTRACT

This invention relates to novel anti-VISTA macrocyclic peptides and their related analogs with appended pharmacokinetic-enhancing tails (PKEs) with general structure of formula (I), which can be used as VISTA inhibitors.

### CROSS REFERENCE TO RELATED APPLICATION

This application claims the benefit of U.S. Provisional Application Ser. No. 63/384,689 filed Nov. 22, 2022 which is incorporated herein in its entirety.

This invention relates to novel anti-VISTA macrocyclic peptides and their related analogs with appended pharmacokinetic-enhancing tails (PKEs) with general structure of formula (I), which can be used as VISTA inhibitors. The macrocyclic peptides described in this invention bind to VISTA, in particular to mouse VISTA and, thus are useful for identification of macrocyclic peptide VISTA tool compounds for animal studies in mice.

### BACKGROUND OF THE INVENTION

Negative immune checkpoints, such as cytotoxic T-lymphocyte-associated protein 4 (CTLA-4) and programmed cell death-ligand 1 (PD-L1) promote cancer growth by downregulation of T-cell activation. Thus, blocking these immune checkpoints restores the ability of immune system to attack cancer cells. FDA-approved monoclonal antibodies (mAbs) against negative immune checkpoints have revealed remarkable clinical success in different malignancies. However, overall response rates to mAbs in cancer immunotherapy are generally lower than 30%. V-domain Ig Suppressor of T-cell Activation (VISTA) is a negative immune checkpoint protein that shares significant homology to PD-L1 in its extracellular domain (ECD). The V-domain Ig-containing suppressor of T-cell activation, or VISTA, is a coinhibitory member of the B7 family of immunoreceptors expressed by myelomonocytic cells and other leukocytes. VISTA has been identified as a potential mediator of resistance to mAb-based immunotherapies in patients based on elevated VISTA levels in patients after administration of anti-CTLA-4 and anti-PD-L1 treatments. Moreover, VISTA has been introduced as a potential immunotherapeutic target in pancreatic cancer due to the engagement of VISTA in diminishing cytokine production in T cells isolated from metastatic pancreatic tumors. Thus, in view of the critical role of VISTA in cancer immunotherapy, it would be advantageous to identify inhibitors of VISTA. Provided herein are macrocyclic peptides that are binds to VISTA, in particular to mouse VISTA.

### SUMMARY OF THE INVENTION

This invention relates to novel anti-VISTA macrocyclic peptides with general structure of formula (I) and their related analogs with appended pharmacokinetic-enhancing tails (PKEs), which can be used as inhibitors of VISTA, in particular of mouse VISTA. The macrocyclic peptides and related analogs described in this invention bind to mouse VISTA and are capable of inhibiting mouse VISTA, thus are useful for identification of macrocyclic peptide VISTA tool compounds for animal studies in nice.

The first aspect of the present invention provides at least one compound of Formula (I)

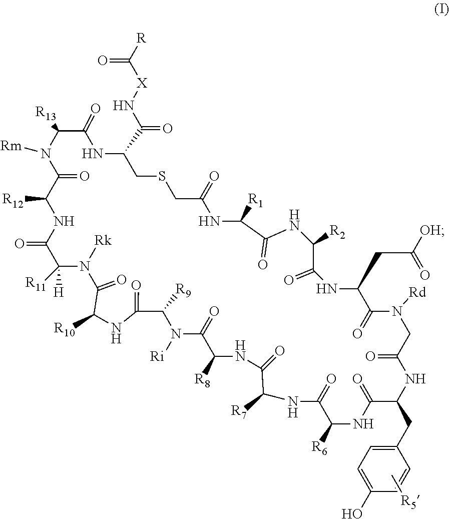

or a pharmaceutically acceptable salt thereof, wherein
R₁ is selected from the group consisting of arylC₁-C₃alkyl, and heteroarylC₁-C₃alkyl; wherein the aryl part of the arylC₁-C₃alkyl is optionally substituted with one, two, or three groups independently selected from halo, nitro, amino, C₁-C₄alkyl, aminocarbonyl, hydroxy, aminoC₁-C₄alkyl, aminoC₂-C₆alkoxy, trifluoromethyl, oxotrifluoromethyl, carboxy, cyano, carboxyC₁-C₄alkyl, and carboxyC₁-C₄alkoxy; and wherein the heteroaryl part of the heteroarylC₁-C₃alkyl is optionally substituted with one, two, or three groups independently selected from C₁-C₃alkyl or halo;
R₂ is selected from the group consisting of arylC₁-C₃alkyl, and heteroarylC₁-C₃alkyl; wherein the aryl part of the arylC₁-C₃alkyl is optionally substituted with one, two, or three groups independently selected from halo, C₁-C₄alkyl, hydroxy, trifluoromethyl, oxotrifluoromethyl, and cyano; and wherein the heteroaryl part of the heteroarylC₁-C₃alkyl is optionally substituted with one, two, or three groups independently selected from C₁-C₃alkyl or halo;
R₅′ is selected from hydrogen or halo;
R₆ is selected from the group consisting of C₁-C₆alkyl, C₃-C₆cycloalkyl, aminoC₃-C₆alkyl, carboxyC₃-C₆alkyl, guanidinylC₃-C₆alkyl, and arylC₁-C₆alkyl; wherein the aryl part of the arylC₁-C₆alkyl is optionally substituted with halo, nitro, hydroxy, carboxy, and carboxyC₁-C₃alkoxy;
R₇ is selected from the group consisting of hydrogen, C₁-C₆alkyl, carboxyC₁-C₄alkyl, aminoC₁-C₄alkyl, and aminocarbonylC₁-C₄alkyl;
R₈ is selected from the group consisting of C₁-C₆alkyl, guanidinylC₃-C₆alkyl, aminoC₁-C₆alkyl, and aminocarbonylaminoC₃-C₆alkyl;
R₉ is selected from the group consisting of hydrogen, C₁-C₆alkyl, guanidinylC₁-C₄alkyl, carboxyC₁-C₄alkyl, hydroxyC₁-C₄alkyl, aminocarbonylC₁-C₄alkyl, aminoC₁-C₄alkyl, arylC₁-C₆alkyl, and heteroarylC₁-C₆alkyl; wherein the aryl part of the arylC₁-C₆alkyl is optionally substituted with halo or hydroxy;
R₁₀ is selected from the group consisting of C₃-C₆alkyl, C₃-C₆cycloalkyl, and phenylC₂-C₄alkyl;
R₁₁ is C₁-C₄alkyl;
R₁₂ is selected from the group consisting of C₃-C₆alkyl, and C₃-C₆cycloalkyl;
R₁₃ is selected from the group consisting of hydrogen, C₁-C₆alkyl, C₃-C₆cycloalkyl, carboxyC₁-C₄alkyl, hydroxyC₁-C₄alkyl, guanidinylC₃-C₆alkyl, aminocarbonylC₁-C₄alkyl; arylC₁-C₃alkyl, and heteroarylC₁-C₃alkyl;
R_{d} is C₄₋₆alkyl or C₂-C₄-aryl;
Rᵢ is hydrogen or C₁-C₆alkyl; or Rᵢ and R₉, together with the carbon atom to which they are attached, form a 5-6 ring heterocycle ring, wherein the heterocycle is optionally fused with an aryl ring;
Rₖ is methyl or Rₖ and R₁₁, together with the carbon atom to which they are attached, form a 4-6 ring heterocycle ring, wherein the heterocycle is optionally substituted with halo, hydroxy or phenyl group;
Rₘ is hydrogen or methyl; or Rₘ and R₁₃, together with the carbon atom to which they are attached, form a 5-6 ring heterocycle ring, wherein the heterocycle is optionally substituted with a hydroxy group;
R is NH₂, OH or NH(CH₂)₁₀₋₁₂COOH;
X is selected from the group consisting of —X₁—, X₁—CONH—X₂—, X₁—CONH—X₂—CONH—X₃—, X₁—CONH—X₂—CONH—X₃—CONH—X₄—, wherein X₁, X₂, X₃, and X₄ is independently selected from CH₂, or any natural or unnatural amino acid side chains, or —(CH₂CH₂O)₂₋₃—; Alternatively X together with COR is a hydrogen.

In one embodiment of the invention, there is disclosed a compound of formula (I), or the pharmaceutically acceptable salt thereof, wherein R₁ is selected from the group consisting of benzyl, naphthyl, or heteroaryl-CH₂; wherein the aryl part of the benzyl group is optionally substituted with one, two, or three groups independently selected from fluoro, chloro, bromo, nitro, amino, C₁-C₃alkyl, aminocarbonyl, hydroxy, aminoC₁-C₄alkyl, aminoC₂-C₆alkoxy, trifluoromethyl, oxotrifluoromethyl, carboxy, cyano, carboxyC₁-C₂alkyl, and carboxymethoxy.

In another embodiment of the invention, there is disclosed a compound of formula (I), or the pharmaceutically acceptable salt thereof, wherein R₂ is selected from the group consisting of benzyl, naphthyl, or heteroaryl-CH₂; wherein the aryl part of the benzyl group is optionally substituted with one or two groups independently selected from fluoro, chloro, C₁-C₃alkyl, hydroxy, trifluoromethyl, and cyano; and wherein the heteroaryl part of the heteroarylC₁-C₃alkyl is optionally substituted with one, two, or three groups independently selected from C₁-C₃alkyl.

In another embodiment of the invention, there is disclosed a compound of formula (I), or the pharmaceutically acceptable salt thereof, wherein R₅′ is selected from hydrogen or chloro.

In another embodiment of the invention, there is disclosed a compound of formula (I), or the pharmaceutically acceptable salt thereof, wherein R₆ is selected from the group consisting of C₂-C₅alkyl, C₃-C₆cycloalkyl, aminoC₃-C₅alkyl, carboxyC₃-C₅alkyl, guanidinylC₃-C₅alkyl, and benzyl; wherein the aryl part of the benzyl group is optionally substituted with fluoro, chloro, nitro, hydroxy, carboxy, and carboxyC₁-C₂alkoxy;

In another embodiment of the invention, there is disclosed a compound of formula (I), or the pharmaceutically acceptable salt thereof, wherein R₇ is selected from hydrogen, C₁-C₄alkyl, carboxyC₁-C₂alkyl, aminoC₁-C₄alkyl, and aminocarbonylC₁-C₂alkyl.

In another embodiment of the invention, there is disclosed a compound of formula (I), or the pharmaceutically acceptable salt thereof, wherein R₈ is selected from the group consisting of C₁-C₄alkyl, guanidinylC₃-C₄alkyl, aminoC₁-C₄alkyl, and aminocarbonylaminoC₃-C₄alkyl.

In another embodiment of the invention, there is disclosed a compound of formula (I), or the pharmaceutically acceptable salt thereof, wherein R₉ is selected from the group consisting of hydrogen, C₁-C₄alkyl, guanidinylC₃-C₄alkyl, carboxyC₁-C₂alkyl, hydroxyC₁-C₄alkyl, aminocarbonylC₁-C₃alkyl, aminoC₁-C₄alkyl, benzyl, and heteroaryl-CH₂; wherein the aryl part of the benzyl group is optionally substituted with hydroxy; alternatively, R₁ and R₉, together with the carbon atom to which they are attached, form a 5-6 ring heterocycle ring, wherein the heterocycle is optionally fused with phenyl ring.

In another embodiment of the invention, there is disclosed a compound of formula (I), or the pharmaceutically acceptable salt thereof, wherein R₁₀ is selected from the group consisting of C₄-C₆alkyl, C₃-C₆cycloalkyl, and phenylC₂-C₄alkyl.

In another embodiment of the invention, there is disclosed a compound of formula (I), or the pharmaceutically acceptable salt thereof, wherein when Rₖ is methyl, R₁₁ is C₁-C₄alkyl; alternatively, Rₖ and R₁₁, together with the carbon atom to which they are attached, form a pyrrolidinyl, azetidinyl, morpholinyl, or piperidinyl ring, wherein the heterocycle is optionally substituted with fluoro, hydroxy or phenyl group.

In another embodiment of the invention, there is disclosed a compound of formula (I), or the pharmaceutically acceptable salt thereof, wherein R₁₂ is selected from the group consisting of C₃-C₄alkyl, and C₃-C₅cycloalkyl.

In another embodiment of the invention, there is disclosed a compound of formula (I), or the pharmaceutically acceptable salt thereof, wherein R₁₃ is selected from the group consisting of hydrogen, C₁-C₄alkyl, C₃-C₅cycloalkyl, carboxyC₁-C₂alkyl, hydroxyC₁-C₃alkyl, guanidinylC₃-C₄alkyl, aminocarbonylC₁-C₄alkyl; benzyl, and heteroaryl-CH₂; or Rₘ and R₁₃, together with the carbon atom to which they are attached, form a pyrrolidinyl or piperidinyl ring, wherein the heterocycle is optionally substituted with a hydroxy group.

In another embodiment of the invention, there is disclosed a compound of formula (I), or the pharmaceutically acceptable salt thereof, wherein R is selected from the group consisting of NH₂, OH, NH(CH₂)₁₀COOH, or NH(CH₂)₁₂COOH.

In another embodiment of the invention, there is disclosed a compound of formula (I), or the pharmaceutically acceptable salt thereof, wherein X₁, X₂, X₃, or X₄ is selected from the group consisting of CH₂, CH(CH₂COOH), CH(CH₂OH), CH(CH₂CH₂COOH), CH(CH₂NH₂), CH(CH₂CH₂NH₂), CH(CH₂CH₂CH₂NH₂), CH(CH₂CH₂CH₂CH₂NH₂), CH(CH₂CONH₂), CH(CH₂CH₂CONH₂), CH(CH₂propargyl), CH(CH₂CH₂CH₂guanidinyl), CH(CH₂(4-hydroxyphenyl)), CH(CH₂indol-3-yl), (CH₂CH₂O)₂, CH(CH₂CH₂)(CH₂CH₂), CH(COOH)CH₂, and CH₂CH(COOH).

In another embodiment of the invention, there is disclosed a compound of formula (I), or the pharmaceutically acceptable salt thereof, wherein
R₁ is benzyl, 2-pyridinylmethyl, 1-napthylmethyl, 2-naphthylmethyl, 4-indolylmethyl, 3-indolylmethyl, or 3-benzothiophenemethyl, 2-methylphenylmethyl, 2-O-allyl-phenylmethyl, 3,4,5-trifluorophenylmethyl, 3,4-dimethoxyphenylmethyl, 3-trifluoromethylphenylmethyl, 3-chlorophenylmethyl, 3-methylphenylmethyl, 3-bromophenylmethyl, 4-trifluoromethylphenylmethyl, 4-methylphenylmethyl, 4-fluorophenylmethyl, 4-iodophenylmethyl, 4-cyanophenylmethyl, 4-aminocarbonylphenylmethyl, 4-aminophenylmethyl, 4-hydroxyphenylmethyl, 4-ethoxyphenylmethyl, 4-O-allylphenylmethyl, 4-methoxyphenylmethyl, and 2,4-difluorophenylmethyl.
R₂ is benzyl, 2-cyanophenylmethyl, 2-O-allyl-phenylmethyl, 3-chlorophenylmethyl, 3-bromophenylmethyl, 3-methylphenylmethyl, 3-cyanophenylmethyl, 3-fluorophenylmethyl, 4-methylphenylmethyl, 4-trifluoromethylphenylmethyl, 4-hydorxyphenylmethyl, 3-indolylmethyl, N-methyl-3-indolylmethyl, and 2,4-difluorophenylmethyl;
R₅′ is hydrogen or chloro;
R₆ is selected from the group consisting of methyl, ethyl, CHMeEt, n-pentyl, isopropyl, n-propyl, isobutyl, n-butyl, cyclopropyl, cyclohexyl, 3-carboxyphenylmethyl, 4-carboxyphenylmethyl, 4-COOH—CH₂O-phenylmethyl, aminobutyl, carboxypropyl, and guanidinylpropyl;
R₇ is selected from the group consisting of hydrogen, methyl, carboxymethyl, carboxyethyl, aminobutyl, and aminocarbonylmethyl;
R₈ is selected from the group consisting of methyl, guanidinylpropyl, aminoethyl, aminopropyl, aminobutyl, and aminocarbonylaminopropyl;
R₉ is selected from the group consisting of hydrogen, methyl, isopropyl, CHMeEt, n-butyl, isobutyl, guanidinylpropyl, carboxymethyl, carboxyethyl, hydroxymethyl, hydroxyCHMe, aminocarbonylmethyl, aminobutyl, 4-carboxyphenylmethyl, 3-carboxyphenylmethyl, 4-hydroxyphenylmethyl, 3-indolylmethyl, 4-COOH—CH₂—O-phenylmethyl; alternatively, when R₁ is n-hexyl, R₉ is hydrogen; alternatively, Rᵢ and R₉, together with the carbon atom to which they are attached, form tetrahydroisoquinolin-3-yl;
R₁₀ is selected from the group consisting of npentyl, cyclopentyl, cyclopropyl, and phenylpropyl;

When Rₖ is methyl, R₁₁ is methyl, n-butyl, or isobutyl, alternatively, Rₖ and R₁₁, together with the carbon atom to which they are attached, form pyrrolidinyl, fluoropyrrolidinyl, hydroxypyrrolidinyl, phenylpyrrolidinyl, azetidinyl, morpholinyl, or piperidinyl ring;
R₁₂ is selected from the group consisting of tert-butyl, isopropyl, C(OH)(CH₃)₃, CH(CH₃)(CH₂CH₃), CH(CH₂CH₃)₂, cyclopropyl, and benzyl;
R₁₃ is selected from the group consisting of hydrogen, methyl, cyclopropyl, npentyl, isopropyl, carboxymethyl, carboxyethyl, hydroxymethyl, OH—CH(CH₃), isobutyl, guanidinylpropyl, aminocarbonylmethyl; benzyl, 4-hydroxyphenylmethyl, and 3-indolylmethyl; Rₘ is hydrogen or methyl; or alternatively, Rₘ and R₁₃, together with the carbon atom to which they are attached, form a pyrrolidinyl or piperidinyl, or hydroxypyrrolidinyl ring;

In another embodiment of the invention, there is disclosed a compound of formula (I), or the pharmaceutically acceptable salt thereof, wherein the compound is selected from the compounds listed in the following tables.

In another embodiment of the invention, there is disclosed a pharmaceutical composition comprising any one of the compounds of the invention, or a pharmaceutically acceptable. salt thereof, for use as an inhibitor of VISTA activity in mice;

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of formula (I) or a pharmaceutically acceptable salt thereof” includes, but is not limited to, a compound of formula (I), two compounds of formula (I), a pharmaceutically acceptable salt of a compound of formula (I), a compound of formula (I) and one or more pharmaceutically acceptable salts of the compound of formula (I), and two or more pharmaceutically acceptable salts of a compound of formula (I).

The term “C₂-C₆alkenyl,” as used herein, refers to a group derived from a straight or branched chain hydrocarbon containing one or more carbon-carbon double bonds containing two to six carbon atoms.

The term “C₁-C₆alkoxy”, as used herein, refers to a C₁-C₆alkyl group attached to the parent molecular moiety through an oxygen atom.

The term “alkyl,” as used herein, refers to a group derived from a straight or branched chain saturated hydrocarbon containing carbon atoms. The term “alkyl” may be proceeded by “C#-C#” wherein the # is an integer and refers to the number of carbon atoms. For example, C₁-C₂alkyl contains one to two carbon atoms and C₁-C₃alkyl contains one to three carbon atoms.

The term “C₁-C₂alkylamino,” as used herein, refers to a group having the formula —NHR, wherein R is a C₁-C₂alkyl group.

The term “C₁-C₂alkylaminoC₁-C₆alkyl,” as used herein, refers to a C₁-C₂alkylamino group attached to the parent molecular moiety through a C₁-C₆alkyl group.

The term “C₁-C₆alkylcarbonyl,” as used herein, refers to a C₁-C₆alkyl group attached to the parent molecular moiety through a carbonyl group.

The term “C₁-C₂alkylcarbonylamino,” as used herein, refers to —NHC(O)Ra, wherein Ra is a C₁-C₆alkyl group.

The term “C₁-C₆alkylcarbonylamino,” as used herein, refers to —NHC(O)Ra, wherein Ra is a C₁-C₂alkyl group.

The term “C₁-C₂alkylcarbonylaminoC₁-C₆alkyl,” as used herein, refers to a C₁-C₂alkylcarbonylamino group attached to the parent molecular moiety through a C₁-C₆alkyl group.

The term “C₁-C₆alkylcarbonylaminoC₁-C₆alkyl,” as used herein, refers to a C₁-C₆alkylcarbonylamino group attached to the parent molecular moiety through a C₁-C₆alkyl group.

The term “C₁-C₆alkylheteroaryl,” as used herein, refers to a heteroaryl group

The term “C₁-C₆alkylheteroarylC₁-C₆alkyl,” as used herein, refers to a C₁-C₆alkylheteroaryl group attached to the parent molecular moiety through a C₁-C₆alkyl group.

The term “C₁-C₆alkylimidazolyl,” as used herein, refers to an imidazolyl ring substituted with one, two, or three C₁-C₆alkyl groups.

The term “C₁-C₆alkylimidazolylC₁-C₂alkyl,” as used herein, refers to a C₁-C₆alkylimidazolyl group attached to the parent molecular moiety through a C₁-C₂alkyl group.

The term “C₂-C₆alkynyl,” as used herein, refers to a group derived from a straight or branched chain hydrocarbon containing one or more carbon-carbon triple bonds containing two to six carbon atoms.

The term “C₂-C₆alkynylmethoxy,” as used herein, refers to a C₂-C₆alkynylmethyl group attached to the parent molecular moiety through an oxygen atom.

The term “C₂-C₆alkynylmethyl,” as used herein, refers to a C₂-C₆alkynyl group attached to the parent molecular moiety through a CH₂ group.

The term “amino,” as used herein, refers to —NH₂.

The term “aminoC₁-C₃alkyl,” as used herein, refers to an amino group attached to the parent molecular moiety through a C₁-C₃alkyl group.

The term “aminoC₁-C₆alkyl,” as used herein, refers to an amino group attached to the parent molecular moiety through a C₁-C₆alkyl group.

The term “aminobutyl,” as used herein, refers to —CH₂CH₂CH₂CH₂NH₂.

The term “aminocarbonyl,” as used herein, refers to an amino group attached to the parent molecular moiety through a carbonyl group.

The term “aminocarbonylC₁-C₂alkyl,” as used herein, refers to an aminocarbonyl group attached to the parent molecular moiety through a C₁-C₂alkyl group.

The term “aminocarbonylC₁-C₃alkyl,” as used herein, refers to an aminocarbonyl group attached to the parent molecular moiety through a C₁-C₃alkyl group.

The term “aminocarbonylC₁-C₆alkyl,” as used herein, refers to an aminocarbonyl group attached to the parent molecular moiety through a C₁-C₆alkyl group.

The term “aminocarbonylamino,” as used herein, refers to an aminocarbonyl group attached to the parent molecular moiety through an amino group.

The term “aminocarbonylaminoC₁-C₆alkyl,” as used herein, refers to an aminocarbonylamino group attached to the parent molecular moiety through a C₁-C₆alkyl group.

The term “aminocarbonylaminoC₂-C₆alkyl,” as used herein, refers to an aminocarbonylamino group attached to the parent molecular moiety through a C₂-C₆alkyl group.

The term “aminocarbonylaminomethyl,” as used herein, refers to an aminocarbonylamino group attached to the parent molecular moiety through a CH₂ group.

The term “aminocarbonylaminopropyl,” as used herein, refers to an aminocarbonylamino group attached to the parent molecular moiety through a CH₂CH₂CH₂ group.

The term “aminocarbonylmethyl,” as used herein, refers to an aminocarbonyl group attached to the parent molecular moiety through a CH₂ group.

The term “aminoethyl,” as used herein, refers to —CH₂CH₂NH₂.

The term “aminomethyl,” as used herein, refers to —CH₂NH₂.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₂alkyl,” as used herein, refers to an aryl group attached to the parent molecular moiety through a C₁-C₂alkyl group.

The term “arylmethyl,” as used herein, refers to an aryl group attached to the parent molecular moiety through a CH₂ group.

The term “carbonyl,” as used herein, refers to —C(O)—.

The term “carboxy”, as used herein, refers to —CO₂H.

The term “carboxyC₁-C₆alkoxy,” as used herein, refers to a carboxyC₁-C₆alkyl group attached to the parent molecular moiety through an oxygen atom.

The term “carboxyC₁-C₆alkyl”, as used herein, refers to a carboxy group attached to the parent molecular moiety through a C₁-C₆alkyl group.

The term “carboxymethoxy,” as used herein, refers to —OCH₂CO₂H.

The term “carboxymethyl,” as used herein, refers to —CH₂CO₂H.

The term “cyano,” as used herein, refers to —CN.

The term “cyanoC₁-C₆alkyl,” as used herein, refers to a cyano group attached to the parent molecular moiety though a C₁-C₆alkyl.

The term “C₃-C₆cycloalkyl”, as used herein, refers to a saturated monocyclic or bicyclic hydrocarbon ring system having three to six carbon atoms and zero heteroatoms. The bicyclic rings can be fused, spirocyclic, or bridged. Representative examples of cycloalkyl groups include, but are not limited to, cyclopropyl, cyclopentyl, and cyclohexyl.

The term “C₃-C₅cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, cyclohexyl, cycloheptyl, and cyclooctyl.

The term “(C₃-C₆cycloalkyl)C₁-C₂alkyl”, as used herein, refers to a C₃-C₆cycloalkyl group attached to the parent molecular moiety through a C₁-C₂alkyl group.

The term “(C₃-C₆cycloalkyl)C₁-C₆alkyl”, as used herein, refers to a C₃-C₆cycloalkyl group attached to the parent molecular moiety through a C₁-C₆alkyl group.

The term “C₃-C₆cycloalkylcarbonyl,” as used herein, refers to a C₃-C₆cycloalkyl group attached to the parent molecular moiety through a carbonyl group.

The term “C₃-C₆cycloalkylcarbonylamino,” as used herein, refers to a C₃-C₆cycloalkylcarbonyl group attached to the parent molecular moiety through an amino group.

The term “C₃-C₆cycloalkylcarbonylaminoC₁-C₆alkyl,” as used herein, refers to a C₃-C₆cycloalkylcarbonylamino group attached to the parent molecular moiety through a C₁-C₆alkyl group.

The term “(C₃-C₆cycloalkyl)methyl”, as used herein, refers to a C₃-C₆cycloalkyl group attached to the parent molecular moiety through a CH₂ group.

The term “cyclopropylcarbonylaminoethyl,” as used herein, refers to —CH₂CH₂NHC(O)R, wherein R is a cyclopropyl group.

The term “difluorocyclohexylmethyl,” as used herein refers to a cyclohexyl group substituted with two fluoro groups that is attached to the parent molecular moiety through a CH₂ group.

The term “ethynylmethoxy,” as used herein, refers to —OCH₂C═CH.

The term “fluoroC₁-C₆alkyl,” as used herein, refers to a C₁-C₆alkyl group substituted by one, two, three, or four fluoro groups.

The term “fluoroC₁-C₆alkylcarbonyl,” as used herein, refers to a fluoroC₁-C₆alkyl group attached to the parent molecular moiety through a carbonyl group.

The term “fluoroC₁-C₆alkylcarbonylamino,” as used herein, refers to a fluoroC₁-C₆alkylcarbonyl group attached to the parent molecular moiety through an NH group.

The term “fluoroC₁-C₆alkylcarbonylaminoC₁-C₆alkyl,” as used herein, refers to a fluoroC₁-C₆alkylcarbonylamino group attached to the parent molecular moiety through a C₁-C₆alkyl group.

The term “fluoroC₄-C₆alkyl,” as used herein, refers to a C₄-C₆alkyl group substituted by one, two, three, or four fluoro groups.

The term “fluoroheterocyclyl,” as used herein, refers to a heterocycle group substituted with one, two, or three fluoro groups.

The term “fluoroheterocyclylC₁-C₆alkyl,” as used herein, refers to a fluoroheterocyclyl group attached to the parent molecular moiety through a C₁-C₆alkyl group.

The term “guanidinylC₁-C₆alkyl,” as used herein, refers to a NH₂C(NH)NH— group attached to the parent molecular moiety through a C₁-C₆alkyl group.

The term “guanidinylC₂-C₄alkyl,” as used herein, refers to a NH₂C(NH)NH— group attached to the parent molecular moiety through a C₂-C₄alkyl group.

The term “guanidinylC₂-C₆alkyl,” as used herein, refers to a NH₂C(NH)NH— group attached to the parent molecular moiety through a C₂-C₆alkyl group.

The terms “halo” and “halogen”, as used herein, refer to F, Cl, Br, or I.

The term “heteroaryl,” as used herein, refers to an aromatic five- or six-membered ring where at least one atom is selected from N, O, and S, and the remaining atoms are carbon.

The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl,” as used herein, refers to a heteroaryl group attached to the parent molecular moiety through a C₁-C₆alkyl group. The term “heteroarylmethyl,” as used herein, refers to a heteroaryl group attached to the parent molecular moiety through a CH₂ group.

The term “heterocyclyl,” as used herein, refers to a five-, six-, or seven-membered non-aromatic ring containing one, two, or three heteroatoms independently selected from nitrogen, oxygen, and sulfur. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure can be attached to the parent molecular moiety through any substitutable atom in the group. Examples of heterocyclyl groups include, but are not limited to, morpholinyl, piperazinyl, pyrrolidinyl, and thiomorpholinyl.

The term “heterocyclylC₁-C₆alkyl,” as used herein, refers to a heterocyclyl attached to the parent molecular moiety through a C₁-C₆alkyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₆alkyl,” as used herein, refers to a hydroxy group attached to the parent molecular moiety through a C₁-C₆alkyl group.

The term “hydroxyaryl,” as used herein, refers to an aryl group substituted with one, two, or three hydroxy groups.

The term “hydroxyarylC₁-C₂alkyl,” as used herein, refers to a hydroxyaryl group attached to the parent molecular moiety through a C₁-C₂alkyl group.

The term “indolylC₁-C₆alkyl,” as used herein, refers to an indolyl group attached to the parent molecular moiety through a C₁-C₆alkyl group.

The term “methoxy,” as used herein, refers to —OCH₃.

The term “methoxyC₁-C₂alkyl,” as used herein, refers to a methoxy group attached to the parent molecular moiety though a C₁-C₂alkyl group.

The term “methylcarbonylamino,” as used herein, refers to —NHC(O)CH₃.

The term “methylcarbonylaminobutyl,” as used herein, refers to —(CH₂)₄NHC(O)CH₃.

The term “methylcarbonylaminobutyl,” as used herein, refers to —(CH₂)₃NHC(O)CH₃.

The term “methylsulfanyl,” as used herein, refers to a —S—CH₃.

The term “methylsulfanylC₁-C₆alkyl,” as used herein, refers to a methylsulfanyl group attached to the parent molecular moiety through a C₁-C₆alkyl group.

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The term “treating” refers to i) inhibiting the disease, disorder, or condition, i.e., arresting its development; and/or ii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

### Peptide Synthesis

The macrocyclic peptides of the present disclosure can be produced by methods known in the art, such as they can be synthesized chemically, recombinantly in a cell free system, recombinantly within a cell or can be isolated from a biological source. Chemical synthesis of a macrocyclic peptide of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic peptides and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-C1-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the peptide analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown below.

### Examples of Orthogonally Protected Amino Acids Used in Solid Phase Synthesis

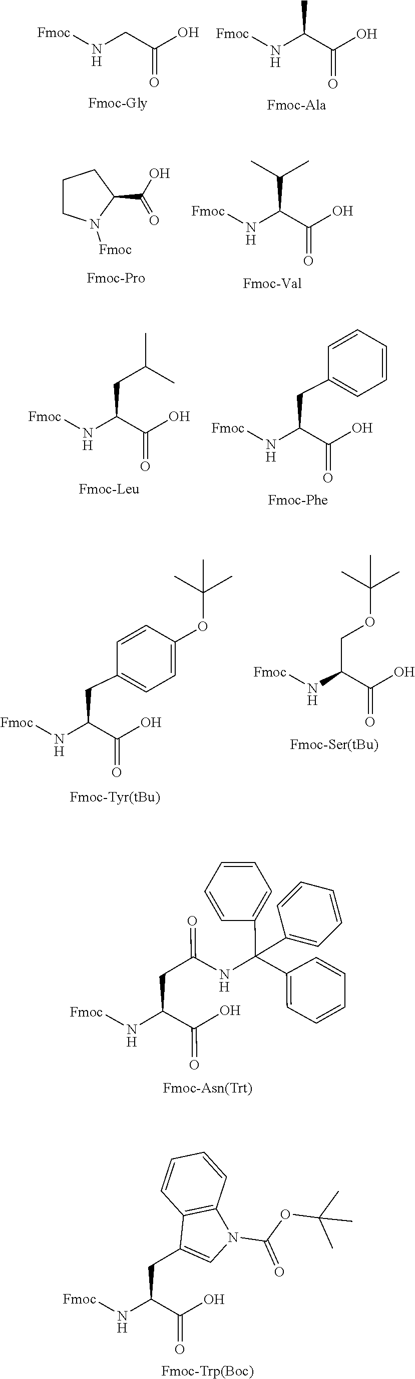

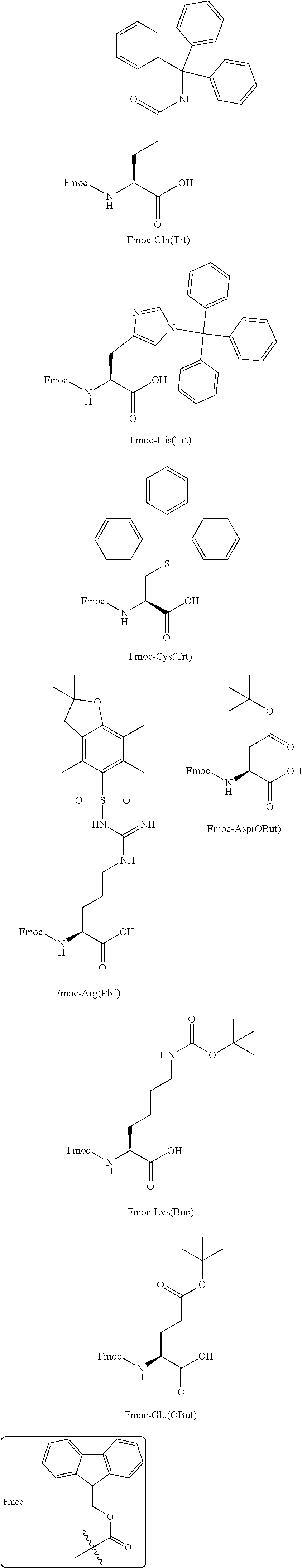

The peptidyl-resin precursors for their respective peptides may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 h at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Peptides with the desired purity can be obtained by purification using preparative HPLC, for example, on a Waters Model 4000 or a Shimadzu Model LC-8A liquid chromatography. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified peptides can be confirmed by electro-spray MS analysis.

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m/z” unit designation. Compounds with exact masses greater than 1000 were often detected as double-charged or triple-charged ions.

The crude material was purified via preparative LC/MS. Fractions containing the desired product were combined and dried via centrifugal evaporation.

### Analytical LC/MS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### The Following Abbreviations are Employed in the Examples and Elsewhere Herein:

| Abbreviation | Full Name |
|---|---|
| Ph | phenyl |
| Bn | benzyl |
| i-Bu | iso-butyl |
| i-Pr | iso-propyl |
| Me | methyl |
| Et | ethyl |
| Pr | n-propyl |
| Bu | n-butyl |
| t-Bu | tert-butyl |
| Trt | trityl |
| TMS | trimethylsilyl |
| TIS | triisopropylsilane |
| Et₂O | diethyl ether |
| HOAc or AcOH | acetic acid |
| MeCN or AcCN | acetonitrile |
| DMF | N,N-dimethylformamide |
| EtOAc | ethyl acetate |
| THF | tetrahydrofuran |
| TFA | trifluoroacetic acid |
| TFE | α,α,α-trifluoroethanol |
| Et₂NH | diethylamine |
| NMM | N-methylmorpholine |
| NMP | N-methylpyrrolidone |
| DCM | dichloromethane |
| TEA | triethylamine |
| min. | minute(s) |
| h or hr | hour(s) |
| L | liter |
| mL or ml | milliliter |
| μL | microliter |
| g | gram(s) |
| mg | milligram(s) |
| mol | mole(s) |
| mmol | millimole(s) |
| meq | milliequivalent |
| rt or RT | room temperature |
| sat or sat'd | saturated |
| aq. | aqueous |
| mp | melting point |
| BOP reagent | benzotriazol-1-yloxy-tris-dimethylamino- |
| | phosphonium hexafluorophosphate |
| | (Castro's reagent) |
| PyBOP reagent | benzotriazol-1-yloxy-tripyrrolidino |
| | phosphonium hexafluorophosphate |
| HBTU | 2-(1H-Benzotriazol-1-yl)-1,1,3,3- |
| | tetramethyluronim hexafluorophosphate |
| HATU | O-(7-Azabenzotriazol-1-yl)-1,1,3,3- |
| | tetramethyluronim hexafluorophosphate |
| HCTU | 2-(6-Chloro-1-H-benzotriazol-1-yl)- |
| | 1,1,3,3-tetramethyluronium |
| | hexafluorophosphate |
| T3P | 2,4,6-tripropyl-1,3,5,2,4,6- |
| | trioxatriphosphorinane-2,4,6-trioxide |
| DMAP | 4-(dimethylamino)pyridine |
| DIEA | diisopropylethylamine |
| Fmoc or FMOC | fluorenylmethyloxycarbonyl |
| Boc or BOC | tert-butyloxycarbonyl |
| HOBT or HOBT•H₂O | 1-hydroxybenzotriazole hydrate |
| Cl-HOBt | 6-Chloro-benzotriazole |
| HOAT | 1-hydroxy-7-azabenzotriazole |
| HPLC | high performance liquid chromatography |
| LC/MS | high performance liquid |
| | chromatography/mass spectrometry |
| MS or Mass Spec | mass spectrometry |
| NMR | nuclear magnetic resonance |
| Sc or SC or SQ | sub-cutaneous |
| IP or ip | intra-peritoneal |

### General Procedures

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis:

Fmoc-Ala-OH; Fmoc-Arg(Pbf)-OH; Fmoc-Asn(Trt)-OH; Fmoc-Asp(tBu)—OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)—OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)—OH; Fmoc-Thr(tBu)—OH; Fmoc-Trp(Boc)-OH; Fmoc-Tyr(tBu)—OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling 4 Equivalent Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling 4 Equivalent Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), then the vessel was closed. The automatic program was resumed and NMM (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure C:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing HATU (an equimolor amount relative to the unnatural amino acid), and NMM (4-8 equiv) was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure D:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing DIC (an equimolor amount relative to the unnatural amino acid), and HOAt (an equimolor amount relative to the unnatural amino acid), was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine:DMF (20:80 v/v, 3.0 mL). The mixture was periodically agitated for 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added bromoacetic acid (0.4 M in DMF, 1.0 mL, 8 eq), then DIC (0.4 M in DMF, 1.0 mL, 8 eq). The mixture was periodically agitated for 1 hour, then the reaction solution was drained through the frit. The resin was washed successively two times as follows: for each wash, DMF (4.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (3.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

Symphony Method: Manipulations were performed under automation on a 12-channel Symphony peptide synthesizer (Protein Technologies) using procedures similar to the ones described for Symphony X.

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-ml bio-rad poly-prep chromatography column was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1, v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH₃CN:H₂O (2:3, v/v, 30 mL), and concentrated to dryness on speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

Cell-Based Binding High-Content Assay For mVISTA

Human embryonic kidney 293T cells (293T) expressing mouse VISTA (mVISTA) were used for the assessment of compounds competing with the binding of a biotinylated peptide. Cryopreserved cells were thawed in a 37° C. water bath and incubated overnight at a 37° C./5% CO₂ incubator in assay culture media DMEM (Life Technologies Inc. Cat. No. 11995-126) supplemented with 10% (v/v) FBS (Sigma, Cat. No. F4135), 1× Penicillin/Streptomycin (Life Technologies Inc. Cat. No. 15140-122). Cells were harvested, washed and resuspended in DMEM media adjusted at. To test compounds under “no-wash” condition, cells were seeded into PDL-coated 384 well plates (Corning, Cat. No. 356663) at 4,000 cells/20 μL. After incubating the cell plates for two hours at a 37° C./5% CO₂ incubator, 125 nL of test compound was added using an ECHO. Alternatively, to test compounds under “wash” condition, test compounds were added first, followed by addition of 20 μL cells at a density of 4000 cells/well. After incubation for an hour, the media was removed, followed by addition of 20 μL of fresh media ( ), and plates were then incubated for another hour. To plates either under “no wash” or “wash” condition, the biotinylated peptide was added to a final concentration of 2 nM, and plates were incubated at room temperature for 30 minutes. Fifteen (15) μL of Alexa 647 conjugated Streptavidin (Life Tech, Cat. No. S21374), suspended at 2 μg/ml in media, was added to the assay plate and incubate for 30 minutes. Cells were fixed by adding 15 μL of formaldehyde at a final concentration of 8% (w/v) (Sigma Cat. No. 252549), 20 μg/mL Hoechst (Thermo Scientific Cat. No. 62249) in PBS for 10 minutes at room temperature. The plates were washed 3 times in dPBS, sealed, and read on a CellInsight NXT High Content Screening Platform IC903000 (Thermo Scientific). The 50% effective concentration (IC50) was calculated using the four-parameter logistic formula y=A+((B−A)/(1+((C/x)\hat{ }D))), where A and B denote minimal and maximal % inhibition, respectively, C is the EC50, D is hill slope and x represent compound concentration.

### Preparation of Example 1000

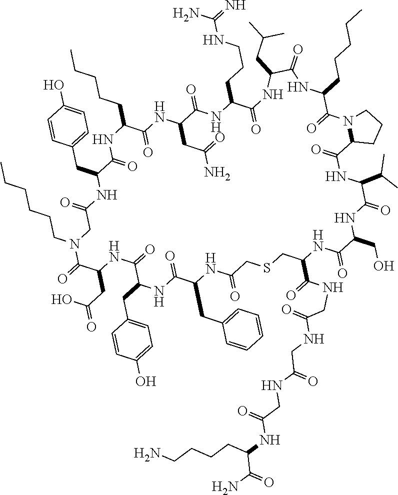 [SMILES: CCCCCCN1CC(=O)N[C@@H](Cc2ccc(O)cc2)C(=O)N[C@@H](CCCCC)C(=O)N[C@H](CC(N)=O)C(=O)N[C@@H](CCCNC(=N)N)C(=O)N[C@@H](CC(C)C)C(=O)N[C@@H](CCCCC)C(=O)N2CCC[C@H]2C(=O)N[C@@H](C(C)C)C(=O)N[C@@H](CO)C(=O)N[C@@H](C(=O)NCC(=O)NCC(=O)NCC(=O)N[C@H](CCCCN)C(N)=O)CSCC(=O)N[C@@H](Cc2ccccc2)C(=O)N[C@@H](Cc2ccc(O)cc2)C(=O)N[C@@H](CC(=O)O)C1=O]

To total 6×10-mL polypropylene solid-phase reaction vessels, each vessel was added rink amide resin (0.56 mmol/g loading) 80 μmol scale, and the reaction vessel was placed on the Symphony X peptide synthesizer. The following procedures were then performed sequentially:
“Symphony X Resin-swelling procedure” was followed;
“Symphony X Single-coupling procedure” was followed with Fmoc-Lys(Boc)-OH;
“Symphony X Single-coupling procedure” was followed with Fmoc-Gly-OH;
“Symphony X Single-coupling procedure” was followed with Fmoc-Gly-OH;
“Symphony X Single-coupling procedure” was followed with Fmoc-Gly-OH;
“Symphony X Single-coupling procedure” was followed with Fmoc-Cys(Trt)-OH;
“Symphony X Single-coupling procedure” was followed with Fmoc-Ser(OtBu)—OH;
“Symphony X Single-coupling procedure” was followed with Fmoc-Val-OH;
“Symphony Double-coupling procedure” was followed with Fmoc-Pro-OH;
“Symphony Double-coupling procedure” was followed with Fmoc-Ahp-OH;
“Symphony Double-coupling procedure” was followed with Fmoc-Leu-OH;
“Symphony X Single-coupling procedure” was followed with Fmoc-Arg(Pbf)-OH;
“Symphony Double-coupling procedure” was followed with Fmoc-Asn(Trt)-OH;
“Symphony X Single-coupling procedure” was followed with Fmoc-Ahp-OH;
“Symphony Double-coupling procedure” was followed with Fmoc-Tyr(tBu)—OH;
“Peptoid Installation Procedure” was followed with bromoacetic acid and hexylamine
“Symphony Double-coupling procedure” was followed with Fmoc-Asp(tBu)—OH;
“Symphony X Single-coupling procedure” was followed with Fmoc-Tyr(tBu)—OH;
“Symphony X Single-coupling procedure” was followed with Fmoc-Phe-OH;
“Symphony X Chloroacetic Anhydride coupling procedure” was followed;
“Global Deprotection Method A” was followed;
“Cyclization Method” was followed.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-45% B over 30 minutes, then a 5-minute hold at 100% B; Flow: 20 mL/min. Fractions containing the desired product were combined and dried via centrifugal evaporation. The yield of the product was 139.4 mg, and its estimated purity by LCMS analysis was 100%.

Retention time=1.68 min;ESI-MS(+)m/z[M+2H]²⁺:1056.2. Analysis condition A

Retention time=1.52 min;ESI-MS(+)m/z[M+2H]²⁺:1055.9. Analysis condition B

The following examples were prepared according to the procedures similar to the one described for Example 1000.

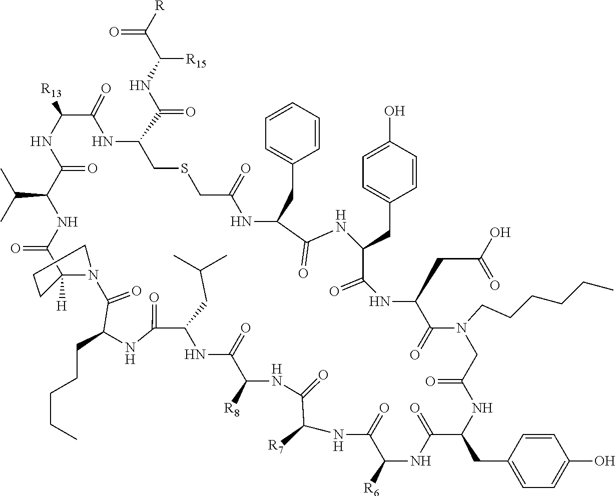

| | | | | | | | | | | | HC |
|---|---|---|---|---|---|---|---|---|---|---|---|
| | | | | | | | | | | | CBA |
| | | | | | | | | QC | Obs. | | mVISTA |
| Ex | | | | | | | Yield | Method | MS | RT | IC50 |
| No | R₆ | R₇ | R₈ | R₁₃ | R₁₅ | R | (mg) | IDs | Ion | (min) | (uM) |
| 1001 | n-butyl | —CH₂COOH | —(CH₂)₃- | CH₂COOH | CH₂COOH | NH2 | 15.5 | A | 1941.1 | 1.32 | 0.0127 |
| | | | guanidinyl | | | | | | | | |
| 1002 | n-pentyl | —CH₂COOH | —(CH₂)₃- | CH₂COOH | —CH₂COOH | NH₂ | 14.1 | B | 1956.4 | 1.75 | 0.0149 |
| | | | guanidinyl | | (d-) | | | | | | |
| 1003 | n-butyl | —CH₂COOH | —(CH₂)₃- | CH₂COOH | —CH₂COOH | NH₂ | 17.8 | A | 1941.3 | 1.31 | 0.0188 |
| | | | guanidinyl | | (d-) | | | | | | |
| 1004 | n-pentyl | —CH₂COOH | —(CH₂)₃- | CH₂COOH | CH₂CH₂ | NH₂ | 4.7 | A | 1969.1 | 1.46 | 0.0129 |
| | | | guanidinyl | | COOH | | | | | | |
| 1005 | n-butyl | —CH₂COOH | —(CH₂)₃- | CH₂COOH | CH₂CH₂ | NH₂ | 5.6 | A | 1954.9 | 1.43 | 0.0127 |
| | | | guanidinyl | | COOH | | | | | | |
| 1006 | n-pentyl | —CH₂CONH₂ | —(CH₂)₃- | methyl | H | NH₂ | 12.5 | A | 1852.1 | 1.67 | 0.0386 |
| | | | guanidinyl | | | | | | | | |
| 1007 | n-butyl | H | —(CH₂)₃- | methyl | H | OH | 6 | B | 1782 | 1.76 | 0.0151 |
| | | | guanidinyl | | | | | | | | |
| 1008 | —CH₂- | —CH₂CONH₂ | —(CH₂)₃- | methyl | H | OH | 21 | B | 1839 | 1.69 | 0.0349 |
| | CH(CH₃)₂ | | guanidinyl | | | | | | | | |
| 1009 | n-butyl | H | —(CH₂)₃- | CH₂COOH | H | OH | 13.9 | A | 1826.1 | 1.48 | 0.0080 |
| | | | guanidinyl | | | | | | | | |
| 1010 | n-butyl | —CH₂COOH | —(CH₂)₃- | CH₂COOH | H | OH | 12.4 | B | 1883.9 | 1.42 | 0.0056 |
| | | | guanidinyl | | | | | | | | |
| 1011 | —CH₂- | —CH₂CONH₂ | —(CH₂)₃- | CH₂COOH | H | OH | 23.8 | A | 1882.8 | 1.43 | 0.0159 |
| | CH(CH₃)₂ | | guanidinyl | | | | | | | | |
| 1012 | —(CH₂)₃- | —CH₂CONH₂ | —(CH₂)₃- | CH₂COOH | H | OH | 22.9 | A | 964.2 | 1.31 | 0.0211 |
| | guanidinyl | | guanidinyl | | | | | | | | |
| 1013 | n-pentyl | CH₂COOH | —(CH₂)₃- | CH₂COOH | H | OH | 17.7 | B | 1899.1 | 1.75 | 0.0171 |
| | | | guanidinyl | | | | | | | | |
| 1014 | —CH₂- | CH₂COOH | —(CH₂)₃- | CH₂COOH | H | NH₂ | 5.1 | A | 1882.7 | 1.44 | 0.0184 |
| | CH(CH₃)₂ | | guanidinyl | | | | | | | | |
| 1015 | n-pentyl | CH₂COOH | —(CH₂)₃- | CH₂COOH | H | NH₂ | 10 | A | 949.2 | 1.4 | 0.0087 |
| | | | guanidinyl | | | | | | | | |
| 1016 | —CH₂- | H | —(CH₂)₃- | CH₂COOH | H | OH | 20.3 | B | 913.1 | 1.6 | 0.0210 |
| | CH(CH₃)₂ | | guanidinyl | | | | | | | | |
| 1017 | n-butyl | H | —(CH₂)₃- | CH₂COOH | H | NH₂ | 20.9 | A | 913.2 | 1.5 | 0.0340 |
| | | | guanidinyl | | | | | | | | |
| 1018 | —CH(CH₃)₂ | H | —(CH₂)₃- | CH₂COOH | H | OH | 19.6 | B | 907.2 | 1.57 | 0.0339 |
| | | | guanidinyl | | | | | | | | |
| 1019 | n-butyl | —CH₂CONH₂ | —(CH₂)₃- | CH₂COOH | H | OH | 28.8 | B | 943.1 | 1.6 | 0.0079 |
| | | | NHCONH₂ | | | | | | | | |
| 1020 | n-butyl | —CH₂CONH₂ | —(CH₂)₃- | —CH₂COOH | H | OH | 6.3 | B | 1883.1 | 1.75 | 0.0225 |
| | | | guanidinyl | | | | | | | | |
| 1021 | n-butyl | —CH₂COOH | —(CH₂)₃- | n-pentyl | H | OH | 24.3 | A | 1896.2 | 1.63 | 0.0322 |
| | | | guanidinyl | | | | | | | | |
| 1022 | n-butyl | H | —(CH₂)₃- | —CH₂CH₂ | H | OH | 8.3 | A | 921.1 | 1.43 | 0.0415 |
| | | | guanidinyl | COOH | | | | | | | |
| 1023 | n-butyl | H | —(CH₂)₃- | H | H | OH | 17.1 | A | 1768.9 | 1.6 | 0.0310 |
| | | | guanidinyl | | | | | | | | |
| 1024 | n-butyl | —CH₂COOH | —(CH₂)₃- | H | H | OH | 15.7 | A | 1826.1 | 1.42 | 0.0136 |
| | | | guanidinyl | | | | | | | | |
| 1025 | n-butyl | —CH₂COOH | —(CH₂)₃- | —CH₂- | H | OH | 15.9 | B | 1882 | 1.68 | 0.0306 |
| | | | guanidinyl | CH(CH₃)₂ | | | | | | | |
| 1026 | n-pentyl | CH₂CONH₂ | —(CH₂)₃- | —CH₂CONH₂ | H | OH | 16.6 | B | 1895.9 | 1.77 | 0.0055 |
| | | | guanidinyl | | | | | | | | |
| 1027 | —CH₂- | CH₂CONH₂ | —(CH₂)₃- | CH₂CONH₂ | H | NH₂ | 21.4 | A | 9413 | 1.67 | 0.0368 |
| | CH(CH₃)₂ | | guanidinyl | | | | | | | | |
| 1028 | n-butyl | H | —(CH₂)₃- | CH₂CONH₂ | H | OH | 11.2 | B | 1825.3 | 1.7 | 0.0052 |
| | | | guanidinyl | | | | | | | | |
| 1029 | n-butyl | —CH₂COOH | —(CH₂)₃- | —CH₂CONH₂ | H | OH | 28.5 | A | 1884 | 1.4 | 0.0011 |
| | | | guanidinyl | | | | | | | | |
| 1030 | —CH₂- | CH₂CONH₂ | —(CH₂)₃- | —(CH₂)₃- | H | OH | 16.5 | A | 1924 | 1.6 | 0.0345 |
| | CH(CH₃)₂ | | guanidinyl | guanidinyl | | | | | | | |
| 1031 | n-pentyl | CH₂CONH₂ | —(CH₂)₃- | —CH₂OH | H | NH₂ | 38.1 | B | 935.2 | 1.64 | 0.0347 |
| | | | guanidinyl | | | | | | | | |
| 1032 | n-pentyl | CH₂CONH₂ | methyl | —CH₂OH | H | NH₂ | 29.1 | A | 1783.1 | 1.64 | 0.0111 |
| 1033 | n-butyl | CH₂CONH₂ | —(CH₂)₃- | —CH₂OH | H | NH₂ | 22.7 | A | 1854.1 | 1.6 | 0.0167 |
| | | | guanidinyl | | | | | | | | |
| 1034 | n-pentyl | CH₂CONH₂ | —(CH₂)₃- | —CH₂OH | H | OH | 29.9 | A | 1870 | 1.64 | 0.0037 |
| | | | guanidinyl | | | | | | | | |
| 1035 | n-pentyl | CH₂CONH₂ | —(CH2)4 | —CH₂OH | H | NH₂ | 28 | A | 1840.1 | 1.62 | 0.0304 |
| | | | NH2 | | | | | | | | |
| 1036 | n-pentyl | CH₂CONH₂ | —(CH2)3 | —CH₂OH | H | NH₂ | 30.3 | B | 1826 | 1.78 | 0.0201 |
| | | | NH2 | | | | | | | | |
| 1037 | n-pentyl | CH₂CONH₂ | —(CH2)2 | —CH₂OH | H | NH₂ | 14.5 | A | 1813 | 1.64 | 0.0364 |
| | | | NH2 | | | | | | | | |
| 1038 | n-pentyl | —CH₂COOH | —(CH₂)₃- | —CH₂OH | H | NH₂ | 12.8 | A | 1869.9 | 1.74 | 0.0384 |
| | | | guanidinyl | | | | | | | | |
| 1039 | n-pentyl | H | —(CH₂)₃- | —CH₂OH | H | NH₂ | 3.4 | A | 1810.9 | 1.91 | 0.0022 |
| | | | guanidinyl | | | | | | | | |
| 1040 | —CH₂- | —CH₂CONH₂ | —(CH₂)₃- | —CH₂OH | H | NH₂ | 4.5 | B | 1854.1 | 1.9 | 0.0054 |
| | CH(CH₃)₂ | | guanidinyl | | | | | | | | |
| 1041 | —CH₂- | —CH₂COOH | —(CH₂)₃- | —CH₂OH | H | NH₂ | 6.8 | A | 1854.9 | 1.64 | 0.0249 |
| | CH(CH₃)₂ | | guanidinyl | | | | | | | | |
| 1042 | n-pentyl | methyl | —(CH₂)₃- | —CH₂OH | H | OH | 14.1 | B | 1826.2 | 1.95 | 0.0363 |
| | | | guanidinyl | | | | | | | | |
| 1043 | —CH₂- | —CH₂CONH₂ | —(CH₂)₃- | —CH₂OH | H | OH | 19.3 | B | 1855.1 | 1.73 | 0.0177 |
| | CH(CH₃)₂ | | guanidinyl | | | | | | | | |
| 1044 | n-pentyl | H | —(CH₂)₃- | —CH₂OH | H | OH | 9.7 | B | 1812.1 | 1.87 | 0.0214 |
| | | | guanidinyl | | | | | | | | |
| 1045 | n-butyl | H | —(CH₂)₃- | —CH₂OH | H | OH | 30.5 | A | 1757.1 | 1.63 | 0.0274 |
| | | | guanidinyl | | | | | | | | |
| 1046 | n-butyl | CH₂CONH₂ | —(CH₂)₃- | —CH₂OH | H | OH | 20.7 | B | 1855.1 | 1.72 | 0.0140 |
| | | | guanidinyl | | | | | | | | |
| 1047 | n-propyl | CH₂CONH₂ | —(CH₂)₃- | —CH₂OH | H | OH | 12.8 | B | 1840.9 | 1.65 | 0.0322 |
| | | | guanidinyl | | | | | | | | |
| 1048 | n-butyl | —CH₂COOH | —(CH₂)₃NH₂ | —CH₂OH | H | NH₂ | 29.3 | A | 1813.1 | 1.6 | 0.0224 |
| 1049 | —CH₂- | H | —(CH₂)₃- | —CH₂OH | H | NH₂ | 9 | B | 1797 | 1.89 | 0.0095 |
| | CH(CH₃)₂ | | guanidinyl | | | | | | | | |
| 1050 | n-butyl | H | —(CH₂)₃- | —CH₂OH | H | NH₂ | 15 | B | 1798 | 1.89 | 0.0299 |
| | | | guanidinyl | | | | | | | | |
| 1051 | —(CH₂)₄NH₂ | —CH₂COOH | —(CH₂)₃NH₂ | —CH₂OH | H | OH | 12.8 | A | 1830.1 | 1.36 | 0.0115 |
| 1052 | —(CH₂)₄NH₂ | —CH₂COOH | —(CH₂)₃- | —CH₂OH | H | OH | 17.7 | B | 1871 | 1.46 | 0.0271 |
| | | | guanidinyl | | | | | | | | |
| 1053 | —(CH₂)₄NH₂ | H | —(CH₂)₃- | —CH₂OH | H | OH | 14.3 | B | 1812.7 | 1.62 | 0.0207 |
| | | | guanidinyl | | | | | | | | |
| 1054 | —CH(CH₃)₂ | H | —(CH₂)₃- | —CH₂OH | H | NH₂ | 16.4 | B | 1784.1 | 1.8 | 0.0297 |
| | | | guanidinyl | | | | | | | | |
| 1055 | —CH(CH₃) | H | —(CH₂)₃- | —CH₂OH | H | NH₂ | 19.8 | B | 1797.3 | 1.86 | 0.0369 |
| | (CH₂CH₃) | | guanidinyl | | | | | | | | |
| 1056 | n-pentyl | —(CH₂)₄ | —(CH₂)₃- | —CH₂OH | H | NH₂ | 8.4 | B | 1882 | 1.85 | 0.0171 |
| | | NH₂ | guanidinyl | | | | | | | | |
| 1057 | n-pentyl | CH₂- | —(CH₂)₃- | —CH₂OH | H | NH₂ | 21 | A | 1883.2 | 1.64 | 0.0351 |
| | | CH₂COOH | guanidinyl | | | | | | | | |
| 1058 | —(CH₂)₄NH₂ | —CH₂CONH₂ | —(CH₂)₃- | —CH₂OH | H | OH | 22.7 | B | 1871.2 | 1.48 | 0.0387 |
| | | | guanidinyl | | | | | | | | |
| 1059 | —CH(CH₃) | H | —(CH₂)₃- | —CH₂OH | H | OH | 17.6 | B | 1799.2 | 1.73 | 0.0300 |
| | (CH₂CH₃) | | guanidinyl | | | | | | | | |
| 1060 | n-propyl | H | —(CH₂)₃- | —CH₂OH | H | NH₂ | 9.7 | B | 1783 | 1.66 | 0.0299 |
| | | | guanidinyl | | | | | | | | |
| 1061 | n-propyl | H | —(CH₂)₃- | —CH₂OH | H | OH | 22.3 | A | 1784.1 | 1.47 | 0.0463 |
| | | | guanidinyl | | | | | | | | |
| 1062 | ethyl | H | —(CH₂)₃- | —CH₂OH | H | OH | 20.8 | A | 886.1 | 1.41 | 0.0152 |
| | | | guanidinyl | | | | | | | | |
| 1063 | —CH(CH₃)₂ | H | —(CH₂)₃- | —CH₂OH | H | OH | 21.9 | A | 1784 | 1.48 | 0.0286 |
| | | | guanidinyl | | | | | | | | |
| 1064 | n-pentyl | H | —(CH₂)₃- | —CH₂OH | H | NH₂ | 21.5 | A | 907 | 1.58 | 0.0093 |
| | | | NHCONH₂ | | | | | | | | |
| 1065 | n-butyl | —CH₂COOH | —(CH₂)₃- | —CH(CH₃)₂ | H | OH | 26.8 | A | 1868.2 | 1.71 | 0.0156 |
| | | | guanidinyl | | | | | | | | |
| 1066 | —CH₂- | —CH₂CONH₂ | —(CH₂)₃- | —CH(CH₃)₂ | H | OH | 20.4 | B | 1866.9 | 1.81 | 0.0024 |
| | CH(CH₃)₂ | | guanidinyl | | | | | | | | |
| 1067 | n-butyl | —CH₂COOH | —(CH₂)₃- | —CH₂- | H | OH | 13.1 | A | 1955 | 1.72 | 0.0289 |
| | | | guanidinyl | indol-3-yl | | | | | | | |
| 1068 | —CH₂- | —CH₂CONH₂ | —(CH₂)₃- | —CH₂- | H | OH | 29.6 | B | 977.9 | 1.74 | 0.0394 |
| | CH(CH₃)₂ | | guanidinyl | indol-3-yl | | | | | | | |
| 1069 | —(CH₂)₃- | —CH₂CONH₂ | —(CH₂)₃- | —CH₂- | H | OH | 20.6 | A | 999.2 | 1.49 | 0.0483 |
| | guanidinyl | | guanidinyl | indol-3-yl | | | | | | | |
| 1070 | n-butyl | H | —(CH₂)₃- | —CH₂- | H | OH | 14.8 | B | 1874.1 | 1.74 | 0.0181 |
| | | | guanidinyl | phenyl-4- | | | | | | | |
| | | | | OH | | | | | | | |
| 1071 | n-butyl | —CH₂COOH | —(CH₂)₃- | —CH₂- | H | OH | 17.1 | A | 1932 | 1.43 | 0.0177 |
| | | | guanidinyl | phenyl-4- | | | | | | | |
| | | | | OH | | | | | | | |
| 1072 | —CH₂- | —CH₂CONH₂ | —(CH₂)₃- | —CH(CH₃)₂ | —CH₂OH | OH | 14.5 | B | 949.3 | 1.75 | 0.0058 |
| | CH(CH₃)₂ | | guanidinyl | | | | | | | | |

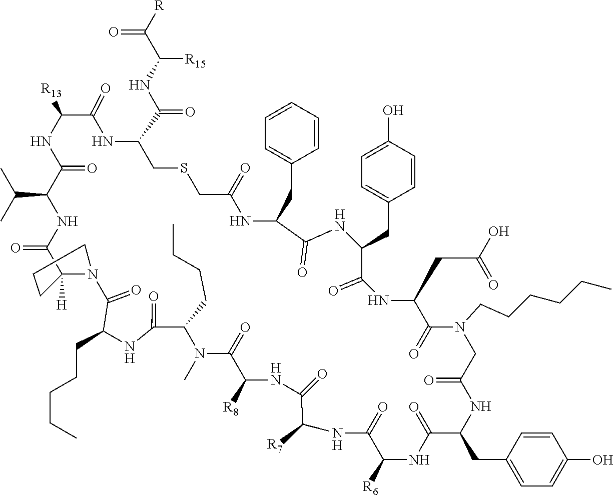

| | | | | | | | | | | | CBA |
|---|---|---|---|---|---|---|---|---|---|---|---|
| | | | | | | | | QC | Obs. | | mVISTA |
| Ex. | | | | | | | Yield | Method | MS | | IC50 |
| No. | R₆ | R₇ | R₈ | R₁₃ | R₁₅ | R | (mg) | IDs | Ion | RT | (uM) |
| 1073 | n-butyl | —CH₂COOH | —(CH₂)₃- | CH₂COOH | CH₂COOH | NH₂ | 18 | A | 1956.3 | 1.3 | 0.0066 |
| | | | guanidinyl | | | | | | | | |
| 1074 | n-pentyl | —CH₂COOH | —(CH₂)₃- | CH₂COOH | CH₂COOH | NH₂ | 14.5 | A | 1969.1 | 1.35 | 0.0097 |
| | | | guanidinyl | | | | | | | | |
| 1075 | n-butyl | CH₂COOH | —(CH₂)₃- | CH₂COOH | —CH₂COOH | NH₂ | 15.6 | B | 1955.3 | 1.67 | 0.0191 |
| | | | guanidinyl | | (d-) | | | | | | |
| 1076 | n-pentyl | CH₂COOH | —(CH₂)₃- | CH₂COOH | CH₂COOH | NH₂ | 17.7 | B | 985.59 | 1.59 | 0.0215 |
| | | | guanidinyl | | (d-) | | | | | | |
| 1077 | n-pentyl | CH₂COOH | —(CH₂)₃- | CH₂COOH | —CH₂CH₂ | NH₂ | 7.5 | A | 1983.3 | 1.49 | 0.0147 |
| | | | guanidinyl | | COOH | | | | | | |
| 1078 | n-butyl | CH₂COOH | —(CH₂)₃- | CH₂COOH | —CH₂CH₂ | NH₂ | 24.1 | A | | 1.24 | 0.0177 |
| | | | guanidinyl | | COOH | | | | | | |
| 1079 | n-pentyl | H | —(CH₂)₃- | —CH₂OH | H | NH₂ | 22.3 | A | 1824.9 | 1.7 | 0.0246 |
| | | | guanidinyl | | | | | | | | |
| 1080 | —CH₂CH | H | —(CH₂)₃- | CH₂OH | H | OH | 32.2 | A | 1813.1 | 1.48 | 0.0186 |
| | (CH₃)₂ | | guanidinyl | | | | | | | | |
| 1081 | —CH₂CH | H | —(CH₂)₃- | CH₂OH | H | NH₂ | 19.8 | B | 1811.9 | 1.72 | 0.0046 |
| | (CH₃)₂ | | guanidinyl | | | | | | | | |
| 1082 | n-butyl | CH₂CONH₂ | —(CH₂)₃- | CH₂COOH | H | OH | 22.1 | A | 950.4 | 1.25 | 0.0081 |
| | | | NHCONH₂ | | | | | | | | |
| 1083 | n-butyl | CH₂CONH₂ | —(CH₂)₃- | CH₂COOH | H | OH | 19.5 | A | 633.2 | 1.32 | 0.0391 |
| | | | guanidinyl | | | | | | | | |

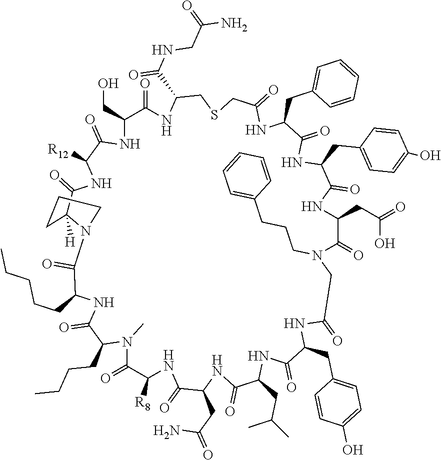

| | | | | QC | Obs. | | |
|---|---|---|---|---|---|---|---|
| Example | | | Yield | Method | MS | RT | HC CBA mVISTA |
| Number | R₈ | R₁₂ | (mg) | IDs | Ior | (min) | IC50 (UM) |
| 1084 | —(CH₂)₃NH₂ | tBu | 31.2 | B | 1874 | 1.64 | 0.0378 |
| 1085 | —(CH₂)₃NH₂ | isopropyl | 45.2 | B | 1860 | 1.62 | 0.0318 |
| 1086 | —(CH₂)₃-guanidinyl | isopropyl | 24.1 | B | 1902 | 1.65 | 0.0407 |
| 1087 | —(CH₂)₃-guanidinyl | tBu | 37.7 | A | 1916.1 | 1.6 | 0.0347 |
| 1088 | —(CH₂)₃NH₂ | tBu | 55 | B | 1826.3 | 1.59 | 0.0402 |

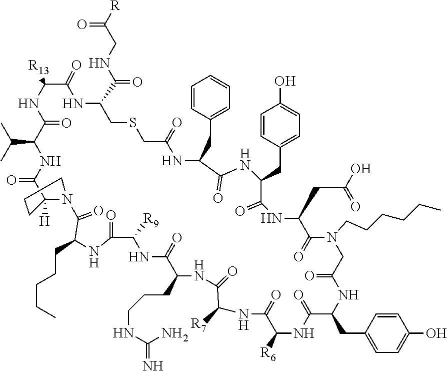

| | | | | | | | | | | HC |
|---|---|---|---|---|---|---|---|---|---|---|
| | | | | | | | | | | CBA |
| | | | | | | | QC | Obs. | | mVISTA |
| Example | | | | | | Yield | Method | MS | | IC50 |
| Number | R₆ | R₇ | R₉ | R₁₃ | R₁₆ | (mg) | IDs | Ion | RT | (uM) |
| 1089 | n-Pentyl | CH₂CONH₂ | methyl | —CH₂OH | NH₂ | 14.8 | A | 1827 | 1.47 | 0.0379 |
| 1090 | n-Pentyl | CH₂CONH₂ | methyl | —CH₂OH | OH | 28.6 | A | 1827.1 | 1.49 | 0.0080 |
| 1091 | CH₂CH(CH₃)₂ | H | —(CH₂)₄NH₂ | —CH₂OH | OH | 19.3 | A | 1813.1 | 1.36 | 0.0053 |
| 1092 | —CH₂CH(CH₃)₂ | H | —(CH₂)₄NH₂ | —CH₂OH | NH₂ | 13.6 | B | 1812 | 1.53 | 0.0271 |
| 1093 | n-Pentyl | H | —CH₂- | —CH₂OH | NH₂ | 12.9 | A | 1861.1 | 1.57 | 0.0404 |
| | | | phenyl-4-OH | | | | | | | |
| 1094 | CH₂CH(CH₃)₂ | H | —CH₂- | —CH₂OH | NH₂ | 20 | B | 1846.8 | 1.65 | 0.0349 |
| | | | phenyl-4-OH | | | | | | | |
| 1095 | CH₂CH(CH₃)₂ | H | —CH₂- | —CH₂OH | OH | 25.5 | B | 1849.1 | 1.54 | 0.0174 |
| | | | phenyl-4-OH | | | | | | | |
| 1096 | CH₂CH(CH₃)₂ | H | —CH₂- | —CH₂COOH | NH₂ | 22.3 | A | 1874.9 | 1.46 | 0.0155 |
| | | | phenyl-4-OH | | | | | | | |
| 1097 | CH₂CH(CH₃)₂ | H | —CH₂- | iPr | OH | 27.4 | B | 1860.1 | 1.62 | 0.0180 |
| | | | phenyl-4-OH | | | | | | | |
| 1098 | n-Pentyl | —CH₂CONH₂ | —CH₂- | —CH₂OH | OH | 11.2 | B | 1943 | 1.77 | 0.0147 |
| | | | indol-3-yl | | | | | | | |

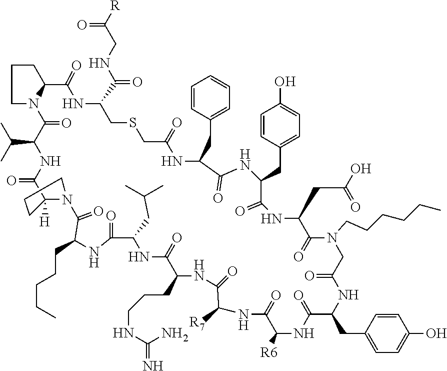

| | | | | | QC | Obs. | | HC CBA |
|---|---|---|---|---|---|---|---|---|
| Example | | | | Yield | Method | MS | | mVISTA |
| Number | R₆ | R₇ | R | (mg) | IDs | Ion | RT | IC50 (uM) |
| 1099 | n-butyl | H | OH | 24 | A | 1808.2 | 1.66 | 0.0216 |
| 1100 | n-pentyl | H | NH₂ | 17.9 | A | 1821 | 1.98 | 0.0176 |
| 1101 | n-butyl | —CH₂COOH | OH | 33.8 | A | 1866.2 | 1.65 | 0.0299 |
| 1102 | n-propyl | H | NH₂ | 8.4 | B | 1792.7 | 1.67 | 0.0257 |
| 1103 | n-propyl | CH₂CONH₂ | OH | 12.3 | B | 618.2 | 1.61 | 0.0026 |
| 1104 | n-butyl | CH₂CONH₂ | OH | 17.5 | A | 933 | 1.46 | 0.0324 |
| 1105 | —CH(CH3)2 | CH₂CONH₂ | OH | 6.2 | A | 926.1 | 1.4 | 0.0376 |
| 1106 | ethyl | H | OH | 13.7 | B | 891 | 1.53 | 0.0254 |
| 1107 | CH₂CH(CH₃)₂ | H | OH | 22.6 | B | 905.2 | 1.62 | 0.0323 |
| 1108 | —CH2CH(CH3)2 | H | NH₂ | 9.1 | B | 904.2 | 1.64 | 0.0335 |
| 1109 | —CH(CH₃)₂ | H | OH | 23.2 | B | 1793.9 | 1.72 | 0.0391 |

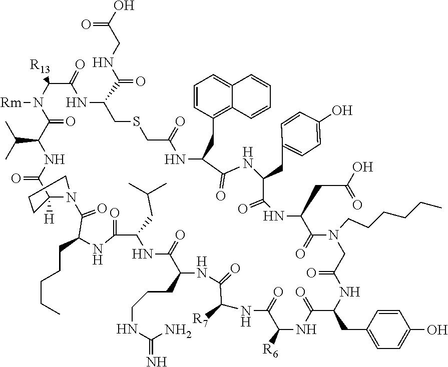

| | | | | | | | | | HC CBA |
|---|---|---|---|---|---|---|---|---|---|
| | | | | | | QC | Obs. | | mVISTA |
| Ex. | | | | | Yield | Method | MS | | IC50 |
| No. | R₆ | R₇ | Rₘ, R₁₃ | R | (mg) | IDs | Ion | RT | (uM) |
| 1110 | iBu | CH₂CONH₂ | n-Hexyl, H | OH | 7.2 | B | 1909 | 2.05 | 0.0368 |
| 1111 | n-Pentyl | H | Hyp | OH | 11.4 | B | 920 | 1.67 | 0.0158 |
| 1112 | n-Bu | CH₂COOH | Me, CH₂Phenyl | OH | 14 | B | 1931.1 | 1.74 | 0.0484 |
| 1113 | n-Bu | H | Me, H | OH | 29.1 | A | 1782 | 1.62 | 0.0352 |
| 1114 | n-Bu | CH₂COOH | Me, H | OH | 14 | B | 1839.8 | 1.56 | 0.0158 |
| 1115 | iBu | CH₂CONH₂ | Me, H | OH | 25.6 | B | 1840 | 1.69 | 0.0069 |
| 1116 | n-Bu | H | CH₂CH₂CH₂- | OH | 24 | A | 1808.2 | 1.66 | 0.0216 |
| 1117 | n-Pentyl | H | CH₂CH₂CH₂- | NH₂ | 17.9 | B | 1821 | 1.98 | 0.0176 |
| 1118 | n-Bu | CH₂COOH | CH₂CH₂CH₂- | OH | 33.8 | A | 1866.2 | 1.65 | 0.0299 |
| 1119 | nPr | H | CH₂CH₂CH₂- | NH₂ | 8.4 | B | 1792.7 | 1.67 | 0.0257 |
| 1120 | nPr | CH₂CONH₂ | CH₂CH₂CH₂- | OH | 12.3 | B | 618.2 | 1.61 | 0.0026 |
| 1121 | n-Bu | CH₂CONH₂ | CH₂CH₂CH₂- | OH | 17.5 | A | 933 | 1.46 | 0.0324 |
| 1122 | iPr | CH₂CONH₂ | CH₂CH₂CH₂- | OH | 6.2 | A | 926.1 | 1.4 | 0.0376 |
| 1123 | Et | H | CH₂CH₂CH₂- | OH | 13.7 | B | 891 | 1.53 | 0.0254 |
| 1124 | iBu | H | CH₂CH₂CH₂- | OH | 22.6 | B | 905.2 | 1.62 | 0.0323 |
| 1125 | iBu | H | CH₂CH₂CH₂- | NH₂ | 9.1 | B | 904.2 | 1.64 | 0.0335 |
| 1126 | iPr | H | CH₂CH₂CH₂- | OH | 23.2 | B | 1793.9 | 1.72 | 0.0391 |

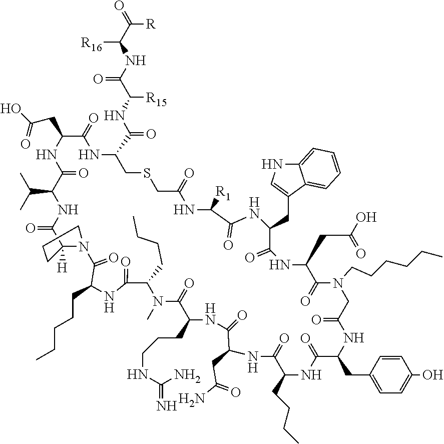

| | | | | | | QC | Obs. | | HC CBA |
|---|---|---|---|---|---|---|---|---|---|
| Ex. | | | | | Yield | Method | MS | | mVISTA |
| No. | R₁ | R₁₅ | R₁₆ | R | (mg) | IDs | Ion | RT | IC50 (uM) |
| 1127 | CH₂- | (CH₂)₄NH₂ | (CH₂)₂CONH₂ | OH | 14.2 | A | 708.04 | 1.38 | 0.0230 |
| | phenyl | | | | | | | | |
| 1128 | CH₂- | (CH₂)₄NH₂ | CH₂CONH₂ | OH | 8.9 | B | 1053 | 1.58 | 0.0157 |
| | phenyl | | | | | | | | |
| 1129 | CH₂- | (CH₂)₃NH₂ | CH₂CONH₂ | OH | 1.3 | B | 1046.4 | 1.54 | 0.0134 |
| | phenyl | | | | | | | | |
| 1130 | CH₂- | (CH₂)₃NH₂ | (CH₂)₂CONH₂ | OH | 3.3 | B | 1053.1 | 1.54 | 0.0175 |
| | phenyl | | | | | | | | |
| 1131 | CH₂- | (CH₂)₂NH₂ | CH₂CONH₂ | OH | 8.2 | B | 693.2 | 1.45 | 0.0231 |
| | phenyl | | | | | | | | |
| 1132 | CH₂- | (CH₂)₂NH₂ | (CH₂)₂CONH₂ | OH | 16.7 | B | 698 | 1.42 | 0.0360 |
| | phenyl | | | | | | | | |
| 1133 | CH₂- | CH₂NH₂ | CH₂CONH₂ | OH | 5.4 | B | 689.2 | 1.43 | 0.0115 |
| | phenyl | | | | | | | | |
| 1134 | CH₂-2- | CH₂NH₂ | (CH₂)₂CONH₂ | OH | 15.8 | B | 714.4 | 1.46 | 0.0137 |
| | naphthyl | | | | | | | | |
| 1135 | CH₂-2- | CH₂NH₂ | (CH₂)₂CONH₂ | OH | 4.8 | B | 710.1 | 1.57 | 0.0391 |
| | naphthyl | | | | | | | | |
| 1136 | CH₂-2- | CH₂NH₂ | CH₂CONH₂ | OH | 7 | B | 705.1 | 1.56 | 0.0095 |
| | naphthyl | | | | | | | | |
| 1137 | CH₂-2- | (CH₂)₄NH₂ | (CH₂)₂CONH₂ | OH | 13.6 | A | 724 | 1.45 | 0.0224 |
| | naphthyl | | | | | | | | |
| 1138 | CH₂-2- | (CH₂)₄NH₂ | CH₂CONH₂ | OH | 4.5 | B | 720.1 | 1.61 | 0.0172 |
| | naphthyl | | | | | | | | |
| 1139 | CH₂-2- | (CH₂)₃NH₂ | CH₂CONH₂ | OH | 1.8 | B | 714.5 | 1.57 | 0.0362 |
| | naphthyl | | | | | | | | |
| 1140 | CH₂-2- | (CH₂)₃NH₂ | (CH₂)₂CONH₂ | OH | 13.2 | B | 719.2 | 1.46 | 0.0191 |
| | naphthyl | | | | | | | | |

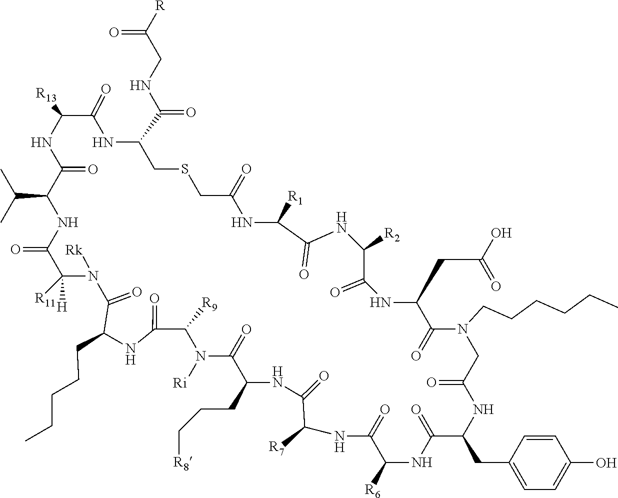

| | | | | | | | | | | | | | | HC |
|---|---|---|---|---|---|---|---|---|---|---|---|---|---|---|
| | | | | | | | | | | | | | | CBA |
| | | | | | | | | | | | | Obs. | | mVISTA |
| Ex. | | | | | | Rᵢ, | | | | Yield | QC | MS | | IC50 |
| No. | R₁ | R₂ | R₆ | R₇ | R_{8′} | R₉ | Rₖ, R₁₁ | R₁₃ | R | (mg) | Method | Ion | RT | (uM) |
| 1141 | CH₂- | CH₂- | nBu | CH₂CONH₂ | NHCONH₂ | Me, | Hyp | CH₂OH | NH₂ | 7.3 | B | 954.8 | 1.58 | 0.0503 |
| | phenyl | indol- | | | | nBu | | | | | | | | |
| | | 3-yl | | | | | | | | | | | | |
| 1142 | CH₂- | CH₂- | nBu | CH₂CONH₃ | NHCONH₂ | Me, | Hyp | CH₂OH | NH₂ | 8.4 | B | 993.2 | 1.93 | 0.0380 |
| | phenyl | indol- | | | | nBu | (Ph) | | | | | | | |
| | | 3-yl | | | | | | | | | | | | |
| 1143 | CH₂- | CH₂- | nBu | CH₂CONH₄ | guanidinyl | Me, | Me, Me | CH₂OH | NH₂ | 10.2 | A | 940.9 | 1.62 | 0.0176 |
| | phenyl | indol- | | | | nBu | | | | | | | | |
| | | 3-yl | | | | | | | | | | | | |
| 1144 | CH₂- | CH₂- | nBu | CH₂CONH₅ | NHCONH₂ | Me, | Me, Me | CH₂OH | NH₂ | 15.9 | B | 1881.2 | 1.79 | 0.0158 |
| | phenyl | indol- | | | | nBu | | | | | | | | |
| | | 3-yl | | | | | | | | | | | | |
| 1145 | CH₂- | CH₂- | nBu | CH₂CONH₆ | guanidinyl | Me, | Pip | CH₂OH | NH₂ | 31.5 | A | 1904.8 | 1.79 | 0.0286 |
| | phenyl | indol- | | | | nBu | | | | | | | | |
| | | 3-yl | | | | | | | | | | | | |
| 1146 | CH₂- | CH₂- | nBu | CH₂CONH₇ | NHCONH₂ | Me, | Pip | CH₂OH | NH₂ | 10.4 | A | 954.1 | 1.61 | 0.0217 |
| | phenyl | indol- | | | | nBu | | | | | | | | |
| | | 3-yl | | | | | | | | | | | | |
| 1147 | CH₂- | CH₂- | nBu | CH₂CONH₈ | guanidinyl | H, | CH₂ | CH₂COOH | OH | 27.8 | A | 1953.9 | 1.48 | 0.0281 |
| | phenyl | indol- | | | | iBu | CH₂CH₂ | | | | | | | |
| | | 3-yl | | | | | | | | | | | | |
| 1148 | CH₂-3- | CH₂4- | iBu | CH₂CONH₉ | guanidinyl | Me, | CH₂ | CH₂OH | NH₂ | 34 | A | 34 | 1.47 | 0.0427 |
| | pyridyl | OHphenyl | | | | nBu | CH₂CH₃ | | | | | | | |
| 1149 | CH₂- | CH₂4- | nBu | CH₂CONH₁₀ | guanidinyl | H, | CH₂ | CH₂COOH | OH | 11.7 | A | 11.7 | 1.57 | 0.0036 |
| | indol-4- | OHphenyl | | | | iBu | CH₂CH₄ | | | | | | | |
| | yl | | | | | | | | | | | | | |
| 1150 | CH₂-3- | CH₂- | nBu | CH₂CONH₁₁ | guanidinyl | H, | CH₂ | CH₂COOH | OH | 28.6 | A | 28.6 | 1.56 | 0.0120 |
| | benzo- | indol- | | | | iBu | CH₂CH₅ | | | | | | | |
| | thiophene | 3-yl | | | | | | | | | | | | |
| 1151 | CH₂-3- | CH₂4- | nBu | CH₂CONH₁₂ | guanidinyl | H, | CH₂₆ | CH₂COOH | OH | 32.3 | A | 32.3 | 1.58 | 0.0057 |
| | benzo- | OHphenyl | | | | iBu | CH₂CH | | | | | | | |
| | thiophene | | | | | | | | | | | | | |
| 1152 | CH₂- | CH₂4- | Ahp | CH₂CONH₁₃ | guanidinyl | H, | Me, Me | CH₂OH | NH₂ | 32.9 | A | 32.9 | 1.72 | 0.0383 |
| | phenyl | OHphenyl | | | | iBu | | | | | | | | |
| 1153 | CH₂- | CH₂4- | Ahp | CH₂CONH₁₄ | guanidinyl | H, | Pip | CH₂OH | NH₂ | 28.9 | A | 28.9 | 1.78 | 0.0346 |
| | phenyl | OHphenyl | | | | iBu | | | | | | | | |
| 1154 | CH₂- | CH₂4- | Ahp | H | guanidinyl | H, | Pip | CH₂OH | OH | 24.3 | A | 24.3 | 1.65 | 0.0126 |
| | phenyl | OHphenyl | | | | iBu | | | | | | | | |
| 1155 | CH₂- | CH₂4- | Ahp | H | guanidinyl | H, | Pip | CH₂OH | NH₂ | 10 | B | 10 | 1.74 | 0.0240 |
| | phenyl | OHphenyl | | | | iBu | | | | | | | | |
| 1156 | CH₂- | CH₂4- | Ahp | CH₂CONH₁₅ | guanidinyl | nHexyl, | CH₂₂ | CH₂CONH₂ | OH | 27.2 | A | 27.2 | 1.58 | 0.0367 |
| | phenyl | OHphenyl | | | | H | CH₂CH | | | | | | | |
| 1157 | CH₂- | CH₂4- | iBu | H | guanidinyl | Tic | CH₂ | CH₂OH | OH | 28 | A | 28 | 1.61 | 0.0144 |
| | phenyl | OHphenyl | | | | | CH₂CH₃ | | | | | | | |
| 1158 | CH₂- | CH₂4- | iBu | H | guanidinyl | Tic | CH₂ | S | NH₂ | 17.6 | A | 184 | 1.73 | 0.0111 |
| | phenyl | OHphenyl | | | | | CH₂CH₄ | | | | | 2.98 | | |

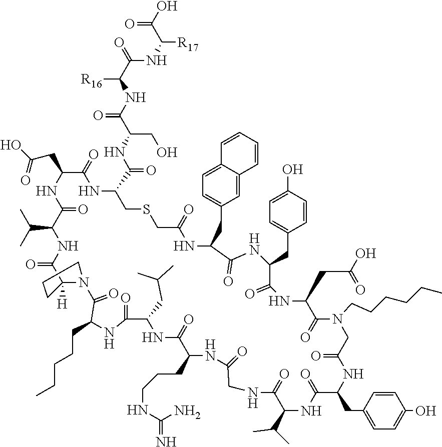

| | | | | | | | HC CBA |
|---|---|---|---|---|---|---|---|
| | | | | | | | mVISTA |
| | | | Yield | QC | Obs. | | IC50 |
| Ex. No. | R₁₆ | R₁₇ | (mg) | Method | MS Ion | RT | (uM) |
| 1159 | (CH₂)₃NH₂ | CH₂CONH₂ | 6.7 | A | 1061.1 | 1.55 | 0.0172 |
| 1160 | (CH₂)₃NH₂ | (CH₂)₂CONH₂ | 18.2 | B | 1067.9 | 1.38 | 0.0371 |
| 1161 | CH₂NH₂ | CH₂CONH₂ | 18.9 | A | 1047.1 | 1.43 | 0.0227 |
| 1162 | CH₂NH₂ | (CH₂)₂CONH₂ | 4.2 | B | 1053.9 | 1.52 | 0.0353 |
| 1163 | (CH₂)₄NH₂ | CH₂CONH₂ | 3.8 | B | 1068.2 | 1.52 | 0.0226 |
| 1164 | (CH₂)₂NH₂ | CH₂CONH₂ | 6.2 | B | 1054.2 | 1.49 | 0.0098 |
| 1165 | (CH₂)₄NH₂ | (CH₂)₂CONH₂ | 14.7 | B | 1075 | 1.4 | 0.0282 |
| 1166 | (CH₂)₂NH₂ | (CH₂)₂CONH₂ | 15.1 | B | 1061 | 1.37 | 0.0188 |

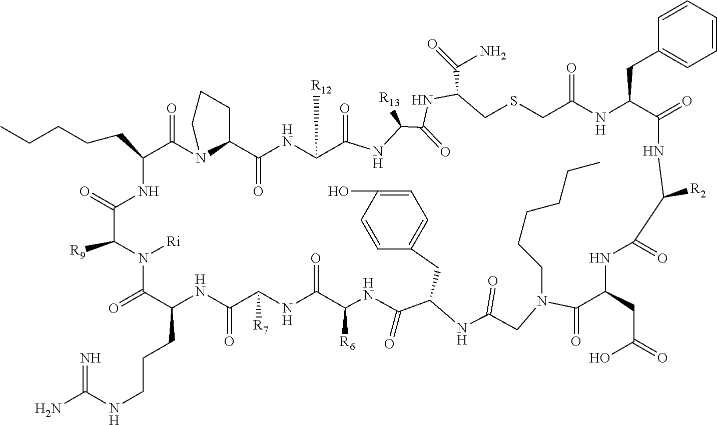

| | | | | | | | | | | | HC |
|---|---|---|---|---|---|---|---|---|---|---|---|
| | | | | | | | | | | | CBA |
| | | | | | | | | QC | Obs. | | mVISTA |
| Ex. | | | | Rᵢ, | | | Yield | Method | MS | | IC50 |
| No. | R₂ | R₆ | R₇ | R₉ | R₁₂ | R₁₃ | (mg) | IDs | Ion | RT | (uM) |
| 1167 | CH₂- | CH₂phenyl | CH₂CONH₂ | Me, | tBu | CH₂COOH | 15.4 | A | 978 | 1.81 | 0.0388 |
| | indol-3- | (4-COOH) | | nBu | | | | | | | |
| | yl | | | | | | | | | | |
| 1168 | CH₂- | CH₂-4- | CH₂CONH₂ | Me, | tBu | CH₂COOH | 6.3 | A | 992.9 | 1.63 | 0.0262 |
| | indol-3- | OCH₂COOH- | | nBu | | | | | | | |
| | yl | phenyl | | | | | | | | | |
| 1169 | CH₂- | (CH₂)₃COOH | CH₂CONH₂ | Me, | tBu | CH₂COOH | 6.3 | A | 1907 | 1.76 | 0.0384 |
| | indol-3- | | | nBu | | | | | | | |
| | yl | | | | | | | | | | |
| 1170 | CH₂- | CH₂-3- | CH₂CONH₂ | Me, | tBu | CH₂COOH | 7.7 | A | 977.9 | 1.37 | 0.0430 |
| | indol-3- | COOH- | | nBu | | | | | | | |
| | yl | phenyl | | | | | | | | | |
| 1171 | CH₂-4- | n-Bu | H | H, | iPr | CH₂COOH | 16.1 | B | 1041.1 | 1.77 | 0.0375 |
| | OHphenyl | | | iBu | | | | | | | |
| 1172 | CH₂-4- | n-Bu | H | H, | iPr | CH₂CONH₂ | 15.2 | B | 1040 | 1.77 | 0.0653 |
| | OHphenyl | | | iBu | | | | | | | |
| 1173 | CH₂-4- | n-pentyl | CH₂CONH₂ | H, | iPr | CH₂OH | 5 | A | 708.3 | 1.7 | 0.0203 |
| | OHphenyl | | | iBu | | | | | | | |

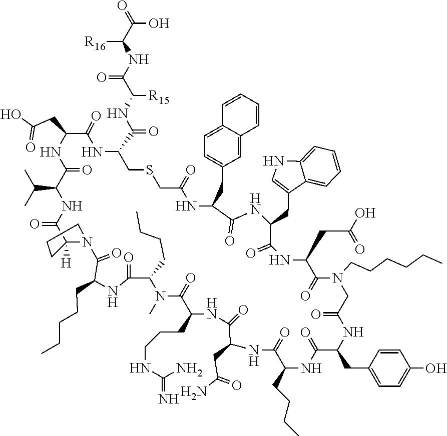

| | | | | QC | | | HC CBA |
|---|---|---|---|---|---|---|---|
| Ex. | | | Yield | Method | Obs. | | mVISTA |
| No. | R₁₅ | R₁₆ | (mg) | IDs | MS Ion | RT | IC50 (uM) |
| 1174 | (CH₂)₂NH₂ | (CH₂)₂CONH₂ | 15.8 | B | 714.4 | 1.46 | 0.0137 |
| 1175 | CH₂NH₂ | (CH₂)₂CONH₂ | 4.8 | B | 710.1 | 1.57 | 0.0391 |
| 1176 | CH₂NH₂ | CH₂CONH₂ | 7 | B | 705.1 | 1.56 | 0.0095 |
| 1177 | (CH₂)₄NH₂ | (CH₂)₂CONH₂ONH₂ | 13.6 | A | 724 | 1.45 | 0.0224 |
| 1178 | (CH₂)₄NH₂ | CH₂CONH₂ | 4.5 | B | 720.1 | 1.61 | 0.0172 |
| 1179 | (CH₂)₃NH₂ | CH₂CONH₂ | 1.8 | B | 714.5 | 1.57 | 0.0362 |
| 1180 | (CH₂)₃NH₂ | (CH₂)₂CONH₂ | 13.2 | B | 719.2 | 1.46 | 0.0191 |

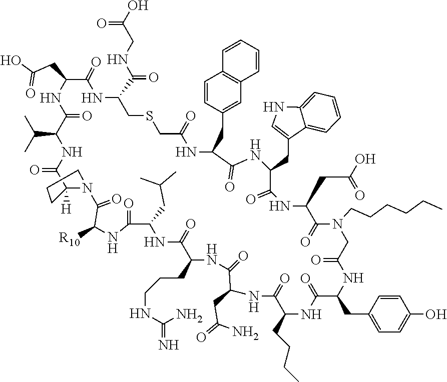

| | | Yield | QC Method | Obs. MS | | |
|---|---|---|---|---|---|---|
| Ex. No. | R₁₀ | (mg) | IDs | Ion | RT | HC CBA mVISTA IC50 (uM) |
| 1181 | cyclopentyl | 16 | B | 977.1 | 1.61 | 0.0159 |
| 1182 | cyclopropyl | 23.1 | A | 964.1 | 1.41 | 0.0236 |

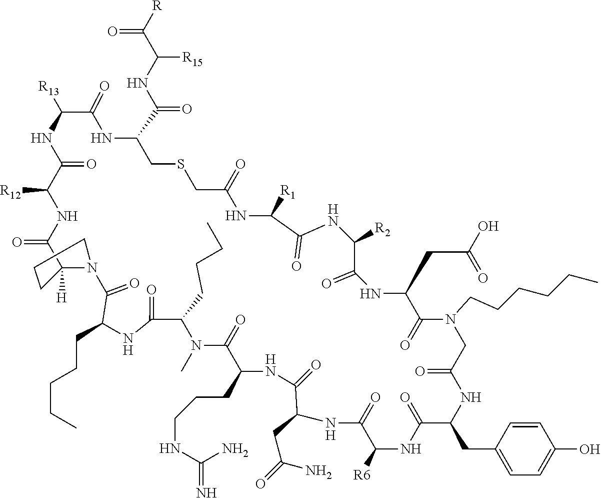

| | | | | | | | | | | | | HC |
|---|---|---|---|---|---|---|---|---|---|---|---|---|
| | | | | | | | | | | | | CBA |
| | | | | | | | | | QC | Obs. | | mVISTA |
| Ex. | | | | | | | | Yield | Method | MS | | IC50 |
| No. | R₁ | R₂ | R₆ | R₁₂ | R₁₃ | R₁₅ | R | (mg) | IDs | Ion | RT | (uM) |
| 1200 | CH₂- | CH₂- | nBu | iPr | CH₂CO | CH₂ | OH | 17.3 | B | 649.5 | 1.85 | 0.0448 |
| | Naphthyl- | 3- | | | OH | | | | | | | |
| | 2-yl | Me- | | | | | | | | | | |
| | | Phenyl | | | | | | | | | | |
| 1201 | CH₂- | CH₂- | n-Pentyl | iPr | CH₂CO | CH₂ | OH | 18.6 | B | 991.5 | 1.91 | 0.0119 |
| | Naphthyl- | 2,4- | | | OH | | | | | | | |
| | 2-yl | diF- | | | | | | | | | | |
| | | phenyl | | | | | | | | | | |
| 1202 | CH₂- | CH₂- | n-Pentyl | iPr | CH₂CO | CH₂OH | OH | 17.1 | B | 1006.4 | 1.83 | 0.0161 |
| | Naphthyl- | 2,4- | | | OH | | | | | | | |
| | 2-yl | diF- | | | | | | | | | | |
| | | phenyl | | | | | | | | | | |
| 1203 | CH₂- | CH₂- | CH₂(CH) | tBu | CH₂OH | H | NH₂ | 45.3 | B | 973.6 | 1.84 | 0.0507 |
| | phenyl | 3-Br- | Me₂ | | | | | | | | | |
| | | phenyl | | | | | | | | | | |
| 1204 | CH₂-2- | CH₂- | CH₂(CH) | iPr | CH₂OH | H | NH₂ | 41.5 | A | 1881.8 | 1.7 | 0.0184 |
| | Me- | 4- | Me₂ | | | | | | | | | |
| | Phenyl | OH- | | | | | | | | | | |
| | | phenyl | | | | | | | | | | |
| 1205 | CH₂-2- | CH₂- | CH₂(CH) | tBu | CH₂OH | H | NH₂ | 34.4 | B | 1895.8 | 1.74 | 0.0164 |
| | Me- | 4- | Me₂ | | | | | | | | | |
| | Phenyl | OH- | | | | | | | | | | |
| | | phenyl | | | | | | | | | | |
| 1206 | CH₂-2- | CH₂- | CH₂(CH) | tBu | CH₂OH | H | NH₂ | 32.2 | A | 1938 | 1.79 | 0.0282 |
| | O-allyl- | 4- | Me₂ | | | | | | | | | |
| | phenyl | OH- | | | | | | | | | | |
| | | phenyl | | | | | | | | | | |
| 1207 | CH₂-3- | CH₂- | CH₂(CH) | tBu | CH₂OH | H | NH₂ | 32.7 | A | 976.1 | 1.62 | 0.0245 |
| | CF₃- | 4- | Me₂ | | | | | | | | | |
| | Phenyl | OH- | | | | | | | | | | |
| | | phenyl | | | | | | | | | | |
| 1208 | CH₂-3- | CH₂- | CH₂(CH) | iPr | CH₂OH | H | NH₂ | 44.2 | A | 1902 | 1.72 | 0.0277 |
| | Cl- | 4- | Me₂ | | | | | | | | | |
| | Phenyl | OH- | | | | | | | | | | |
| | | phenyl | | | | | | | | | | |
| 1209 | CH₂-3- | CH₂- | CH₂(CH) | tBu | CH₂OH | H | NH₂ | 63.5 | A | 1916 | 1.76 | 0.0332 |
| | Cl- | 4- | Me₂ | | | | | | | | | |
| | Phenyl | OH- | | | | | | | | | | |
| | | phenyl | | | | | | | | | | |
| 1210 | CH₂-3 | CH₂- | CH₂(CH) | tBu | CH₂OH | H | NH₂ | 30.9 | B | 1906.8 | 1.68 | 0.0383 |
| | CN- | 4- | Me₂ | | | | | | | | | |
| | Phenyl | OH- | | | | | | | | | | |
| | | phenyl | | | | | | | | | | |
| 1211 | CH₂-3- | CH₂- | nBu | iPr | CH₂COOH | H | OH | 14.8 | B | 955.7 | 1.8 | 0.0221 |
| | Me- | 4- | | | | | | | | | | |
| | Phenyl | OH- | | | | | | | | | | |
| | | phenyl | | | | | | | | | | |
| 1212 | CH₂-4- | CH₂- | CH₂(CH) | tBu | CH₂OH | H | NH₂ | 47.5 | B | 1925 | 1.53 | 0.0131 |
| | CONH₂- | 4- | Me₂ | | | | | | | | | |
| | Phenyl | OH- | | | | | | | | | | |
| | | phenyl | | | | | | | | | | |
| 1213 | CH₂-4- | CH₂- | CH₂(CH) | iPr | CH₂OH | H | NH₂ | 29.9 | A | 1912 | 1.42 | 0.0271 |
| | CONH₂- | 4- | Me₂ | | | | | | | | | |
| | Phenyl | OH- | | | | | | | | | | |
| | | phenyl | | | | | | | | | | |
| 1214 | CH₂-4- | CH₂- | CH₂(CH) | iPr | CH₂OH | H | NH₂ | 49.6 | A | 1882 | 1.72 | 0.0233 |
| | Me- | 4- | Me₂ | | | | | | | | | |
| | Phenyl | OH- | | | | | | | | | | |
| | | phenyl | | | | | | | | | | |
| 1215 | CH₂-4- | CH₂- | CH₂(CH) | tBu | CH₂OH | H | NH₂ | 36.5 | A | 1895.9 | 1.76 | 0.0167 |
| | Me- | 4- | Me₂ | | | | | | | | | |
| | Phenyl | OH- | | | | | | | | | | |
| | | phenyl | | | | | | | | | | |
| 1216 | CH₂- | CH₂- | n-Pentyl | iPr | CH₂COOH | H | OH | 48.7 | B | 984.2 | 1.78 | 0.0129 |
| | 2,4- | 2,4- | | | | | | | | | | |
| | diF- | diF- | | | | | | | | | | |
| | phenyl | phenyl | | | | | | | | | | |
| 1217 | CH₂- | CH₂- | n-Pentyl | iPr | iPr | H | OH | 45.6 | A | 975.9 | 1.81 | 0.0162 |
| | 2,4- | 2,4- | | | | | | | | | | |
| | diF- | diF- | | | | | | | | | | |
| | phenyl | phenyl | | | | | | | | | | |
| 1218 | CH₂- | CH₂- | n-Pentyl | iPr | CH₂COOH | CH₂OH | OH | 11.5 | B | 999.1 | 1.89 | 0.0236 |
| | 2,4 | 2,4- | | | | | | | | | | |
| | diF- | diF- | | | | | | | | | | |
| | phenyl | phenyl | | | | | | | | | | |
| 1219 | CH₂- | CH₂- | n-Pentyl | iPr | iPr | CH₂OH | OH | 16.7 | A | 991.7 | 1.69 | 0.0339 |
| | 2,4- | 2,4- | | | | | | | | | | |
| | diF- | diF | | | | | | | | | | |
| | phenyl | phenyl | | | | | | | | | | |
| 1220 | CH₂- | CH₂- | n-Pentyl | iPr | iPr | H | OH | 24 | B | 1954.1 | 1.76 | 0.0247 |
| | 2,4- | indol- | | | | | | | | | | |
| | diF- | 3-yl | | | | | | | | | | |
| | phenyl | | | | | | | | | | | |
| 1221 | CH₂- | CH₂- | n-Pentyl | iPr | iPr | CH₂OH | OH | 20.6 | B | 993.2 | 1.87 | 0.0203 |
| | 2,4- | indol- | | | | | | | | | | |
| | diF- | 3-yl | | | | | | | | | | |
| | phenyl | | | | | | | | | | | |
| 1222 | CH₂-4- | CH₂- | CH₂(CH) | iPr | CH₂OH | H | NH₂ | 31.9 | A | 1923.8 | 1.75 | 0.0217 |
| | O-allyl- | 4- | Me2 | | | | | | | | | |
| | phenyl | OH- | | | | | | | | | | |
| | | phenyl | | | | | | | | | | |
| 1223 | CH₂-4- | CH₂- | CH₂(CH) | tBu | CH₂OH | H | NH₂ | 35.6 | B | 1937.8 | 1.79 | 0.0181 |
| | O-allyl- | 4- | Me2 | | | | | | | | | |
| | phenyl | OH- | | | | | | | | | | |
| | | phenyl | | | | | | | | | | |
| 1224 | CH₂-3- | CH₂- | CH₂(CH) | iPr | CH₂OH | H | NH₂ | 3.2 | B | 974.1 | 1.66 | 0.0226 |
| | Br- | 4- | Me2 | | | | | | | | | |
| | phenyl | OH- | | | | | | | | | | |
| | | phenyl | | | | | | | | | | |
| 1225 | CH₂-3- | CH₂- | CH₂(CH) | tBu | CH₂OH | H | NH₂ | 6.1 | A | 981.1 | 1.61 | 0.0312 |
| | Br- | 4- | Me2 | | | | | | | | | |
| | phenyl | OH- | | | | | | | | | | |
| | | phenyl | | | | | | | | | | |

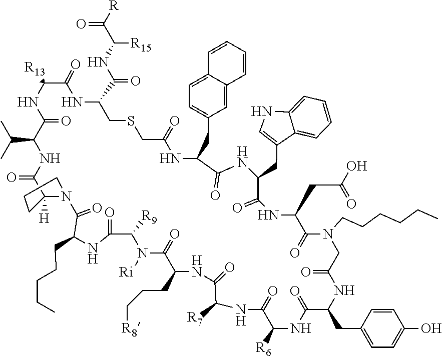

| | | | | | | | | | | | | HC |
|---|---|---|---|---|---|---|---|---|---|---|---|---|
| | | | | | | | | | | | | CBA |
| Ex. | | | | | | | | | QC | Obs. | | mVISTA |
| Num | | | | | | | | Yield | Method | MS | | IC50 |
| ber. | R₆ | R₇ | R₈ | Rᵢ, R₉ | R₁₃ | R₁₅ | R | (mg) | IDs | Ion | RT | (uM) |
| 1226 | nBu | CH₂COOH | guanidinyl | H, iBu | CH₂COOH | CH₂COOH | NH₂ | 12 | B | 1008 | 1.78 | 0.0144 |
| 1227 | nBu | CH₂COOH | NH2 | H, iBu | CH₂COOH | CH₂COOH | NH₂ | 4.9 | A | 1971.9 | 1.6 | 0.0322 |
| 1228 | iPr | H | guanidinyl | H, iBu | CH₂COOH | H | NH₂ | 5.8 | B | 942.2 | 1.67 | 0.0271 |
| 1229 | nBu | CH₂CONH₂ | guanidinyl | H, iBu | CH₂COOH | H | OH | 14.5 | A | 978.3 | 1.57 | 0.0132 |
| 1230 | iPr | H | guanidinyl | H, iBu | (CH₂)₃- | H | OH | 5.5 | A | 1926 | 1.67 | 0.0057 |
| | | | | | guanidinyl | | | | | | | |
| 1231 | iBr | H | guanidinyl | H, iBu | (CH₂)₃- | H | NH₂ | 3.9 | A | 1937.3 | 1.85 | 0.0096 |
| | | | | | guanidinyl | | | | | | | |
| 1232 | nBu | CH₂CONH₂ | guanidinyl | H, iBu | cPr | H | OH | 20.4 | A | 970 | 1.69 | 0.0360 |
| 1233 | nBu | CH₂CONH₂ | NHCONH2 | H, iBu | CH₂COOH | H | OH | 17 | B | 979.3 | 1.71 | 0.0105 |
| 1234 | nBu | CH₂CONH₂ | guanidinyl | H, iBu | CH₂COOH | CH₂- | OH | 16.9 | B | 998.2 | 1.75 | 0.0430 |
| 1235 | nBu | CH₂COOH | guanidinyl | H, iBu | CH₂COOH | CH₂OH | NH₂ | 15.2 | A | 994.63, | 1.48, | 0.0134 |
| | | | | | | | | | | 993.82 | 1.53 | |
| 1236 | nBu | CH₂COOH | NH2 | H, iBu | CH₂COOH | CH₂OH | NH₂ | 15.5 | A | 973 | 1.57, | 0.0277 |
| | | | | | | | | | | | 1.62 | |
| 1237 | nBu | CH₂CONH₂ | guanidinyl | Me, | CH₂COOH | H | OH | 16.1 | B | 1971.1 | 1.57 | 0.0091 |
| | | | | CHMe | | | | | | | | |
| | | | | Et | | | | | | | | |
| 1238 | nBu | CH₂CONH₂ | guanidinyl | Me, | CH₂COOH | H | OH | 13.3 | B | 1970.8 | 1.38 | 0.0252 |
| | | | | CH₂COOH | | | | | | | | |
| 1239 | nBu | CH₂CONH₂ | guanidinyl | Me, iBu | CH₂COOH | H | OH | 13.5 | B | 1969.9 | 1.62 | 0.0032 |
| 1240 | nBu | CH₂CONH₂ | guanidinyl | Me, iBu | CH₂COOH | CH₂OH | OH | 17.6 | A | 1001.16, | 1.42, | 0.0144 |
| | | | | | | | | | | 1001.1 | 1.45 | |
| 1241 | nBu | CH₂CONH₂ | guanidinyl | Me, | CH₂COOH | H | OH | 14.4 | A | 1969.7 | 1.36 | 0.0351 |
| | | | | CH₂CONH₂ | | | | | | | | |
| 1242 | nBu | CH₂COOH | NHCONH2 | Me, nBu | CH₂COOH | CH₂COOH | NH₂ | 17.2 | A | 1015.3 | 1.36 | 0.0089 |
| 1243 | nBu | CH₂COOH | guanidinyl | Me, nBu | CH₂COOH | CH₂COOH | NH₂ | 5 | A | 1014.5 | 1.61 | 0.0201 |
| 1244 | nBu | CH₂COOH | NH2 | Me, nBu | CH₂COOH | CH₂COOH | NH₂ | 11.2 | A | 1986 | 1.56 | 0.0135 |
| 1245 | nBu | CH₂CONH₂ | guanidinyl | Me, nBu | CH₂COOH | H | OH | 29.4 | B | 1970 | 1.61 | 0.0062 |
| 1246 | nBu | CH₂CONH₂ | guanidinyl | Me, | (CH₂)₃- | H | OH | 21.5 | B | 1006.1 | 1.76 | 0.0251 |
| | | | | nBu | guanidinyl | | | | | | | |
| 1247 | nBu | CH₂CONH₂ | guanidinyl | Me, | CH₂OH | H | OH | 26 | B | 1942.1 | 1.73 | 0.0352 |
| | | | | nBu | | | | | | | | |
| 1248 | nBu | CH₂CONH₂ | guanidinyl | Me, | iPr | H | OH | 12.8 | B | 1954.1 | 1.81 | 0.0384 |
| | | | | nBu | | | | | | | | |
| 1249 | nBu | CH₂CONH₂ | guanidinyl | Me, | CH₂CONH₂ | H | OH | 33.7 | A | 1969 | 1.57 | 0.0139 |
| | | | | nBu | | | | | | | | |
| 1250 | nPentyl | CH₂CONH₂ | guanidinyl | Me, | iPr | H | OH | 42.7 | B | 985.1 | 2.02 | 0.0221 |
| | | | | nBu | | | | | | | | |
| 1251 | nPentyl | CH₂CONH₂ | guanidinyl | Me, | CH₂COOH | H | OH | 43.9 | A | 992.9 | 1.61 | 0.0125 |
| | | | | nBu | | | | | | | | |
| 1252 | nPentyl | CH₂CONH₂ | guanidinyl | Me, | CH2OH | H | OH | 23.2 | A | 979.3 | 1.63 | 0.0064 |
| | | | | nBu | | | | | | | | |
| 1253 | nBu | CH₂CONH₂ | NHCONH₂ | Me, | CH₂COOH | H | OH | 27.2 | B | 658.3 | 1.73 | 0.0069 |
| | | | | nBu | | | | | | | | |
| 1254 | nPentyl | CH₂CONH₂ | guanidinyl | Me, | CH₂COOH | CH₂OH | NH₂ | 43 | A | 1007.1 | 1.61 | 0.0157 |
| | | | | nBu | | | | | | | | |
| 1255 | nBu | CH₂CONH₂ | guanidinyl | Me, | CH₂COOH | CH₂OH | OH | 13.3 | B | 1001.2 | 1.73 | 0.0131 |
| | | | | nBu | | | | | | | | |
| 1256 | nBu | CH₂COOH | NH2 | Me, | CH₂COOH | CH₂OH | NH₂ | 11.9 | B | 980 | 1.71 | 0.0129 |
| | | | | nBu | | | | | | | | |
| 1257 | nBu | CH₂COOH | guanidinyl | Me, | CH₂COOH | CH₂OH | NH₂ | 7.6 | A | 1001.2 | 1.45 | 0.0113 |
| | | | | nBu | | | | | | | | |
| 1258 | nBu | CH₂CONH₂ | guanidinyl | Me, | CH₂COOH | H | OH | 30.5 | A | 1006.6 | 1.52 | 0.0120 |
| | | | | (CH₂)₃- | | | | | | | | |
| | | | | guanidinyl | | | | | | | | |
| 1259 | nBu | CH₂CONH₂ | guanidinyl | Me, | CH₂COOH | H | OH | 24.8 | A | 1945.2 | 1.6 | 0.0080 |
| | | | | CH₂OH | | | | | | | | |
| 1260 | nBu | CH₂CONH₂ | guanidinyl | Me, iPr | CH₂COOH | H | OH | 9.5 | B | 1956.2 | 1.54 | 0.0094 |
| 1261 | nBu | CH₂CONH₂ | guanidinyl | Me, | CH₂COOH | H | OH | 10.7 | A | 2043.4 | 1.5 | 0.0117 |
| | | | | CH₂- | | | | | | | | |
| | | | | indol- | | | | | | | | |
| | | | | 3-yl | | | | | | | | |
| 1262 | nBu | CH₂CONH₂ | guanidinyl | CH₂, | CH₂COOH | H | OH | 22.3 | A | 1010.2 | 1.43 | 0.0216 |
| | | | | CH₂-4- | | | | | | | | |
| | | | | OH- | | | | | | | | |
| | | | | phenyl | | | | | | | | |
| 1263 | iBu | H | guanidinyl | H, | CH₂COOH | H | OH | 23.1 | A | 1948.9 | 1.45 | 0.0200 |
| | | | | CH₂-4- | | | | | | | | |
| | | | | OH- | | | | | | | | |
| | | | | phenyl | | | | | | | | |
| 1264 | iBu | H | guanidinyl | H, | CH₂COOH | H | NH₂ | 18.8 | A | 1948.3 | 1.56 | 0.0284 |
| | | | | CH₂-4- | | | | | | | | |
| | | | | OH- | | | | | | | | |
| | | | | phenyl | | | | | | | | |

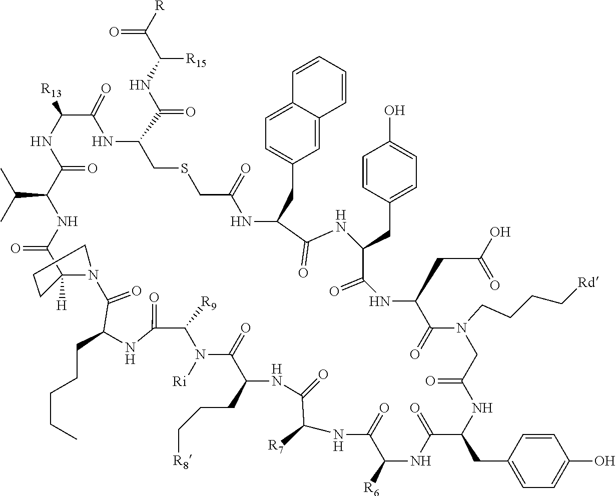

| | | | | | | | | | | | | | HC |
|---|---|---|---|---|---|---|---|---|---|---|---|---|---|
| | | | | | | | | | | | | | CBA |
| Ex | | | | | | | | | | QC | Obs. | | mVISTA |
| Num | | | | | | | | | Yield | Method | MS | | IC50 |
| ber. | R₄ | R₆ | R₇ | R₈ | Rᵢ, R₉ | R₁₃ | R₁₅ | R₁₆ | (mg) | IDs | Ion | RT | (uM) |
| 1265 | Et | nBu | CH₂COOH | NHCONH₂ | Me, | CH₂COOH | CH₂COOH | NH₂ | 5.8 | B | 1004.4 | 1.63 | 0.009 |
| | | | | | nBu | | | | | | | | |
| 1266 | Et | nBu | CH₂COOH | guanidinyl | Me, | CH₂COOH | CH₂COOH | NH₂ | 11.6 | B | 1003.73 | 1.61 | 0.0065 |
| | | | | | nBu | | | | | | | | |
| 1267 | Et | nBu | CH₂COOH | guanidinyl | H, iBu | CH₂COOH | CH₂COOH | NH₂ | 9.3 | A | 1991.1 | 1.54 | 0.0153 |
| 1268 | Et | nBu | CH₂COOH | NH₂ | H, iBu | CH₂COOH | CH₂COOH | NH₂ | 16.4 | B | 1949 | 1.78 | 0.0097 |
| 1269 | H | iPr | H | guanidinyl | H, iBu | CH₂COOH | H | OH | 17.9 | A | 918.1 | 1.31 | 0.0157 |
| 1270 | H | nBu | CH₂CONH₂ | guanidinyl | H, iBu | CH₂COOH | H | OH | 16.4 | A | 953.4 | 1.33 | 0.0024 |
| 1271 | H | nBu | CH₂CONH₂ | guanidinyl | H, iBu | CH₂COOH | H | NH₂ | 7.7 | A | 953.1 | 1.46 | 0.0080 |
| 1272 | Et | nBu | CH₂CONH₂ | guanidinyl | H, iBu | CH₂OH | H | OH | 28.1 | B | 1905 | 1.82 | 0.0028 |
| 1273 | Et | nPentyl | H | guanidinyl | H, iBu | CH₂OH | H | NH₂ | 19.2 | B | 621 | 1.77 | 0.0143 |
| 1274 | Et | iBu | H | guanidinyl | H, iBu | CH₂OH | H | OH | 20.3 | A | 924.1 | 1.61 | 0.0320 |
| 1275 | Et | iBu | H | guanidinyl | H, iBu | CH₂OH | H | NH₂ | 23.8 | A | 1847.1 | 1.7 | 0.0359 |
| 1276 | Et | Nle | H | guanidinyl | H, iBu | CH₂OH | H | NH₂ | 8 | B | 924.1 | 1.7 | 0.0406 |
| 1277 | Et | iPr | H | guanidinyl | H, iBu | CH₂OH | H | OH | 11 | B | 1835.1 | 1.76 | 0.0232 |
| 1278 | Et | iBu | CH₂COOH | guanidinyl | H, iBu | CH₂COOH | H | OH | 12.4 | A | 967.1 | 1.33 | 0.0240 |
| 1279 | Et | nPentyl | CH₂COOH | guanidinyl | H, iBu | CH₂COOH | H | NH₂ | 17.9 | A | 974.1 | 1.53 | 0.0266 |
| 1280 | Et | iPr | H | guanidinyl | H, iBu | CH₂COOH | H | OH | 20.2 | B | 931.1 | 1.63 | 0.0337 |
| 1281 | Et | iBu | H | guanidinyl | H, iBu | CH₂COOH | H | NH₂ | 14.9 | B | 938.4 | 1.59 | 0.0229 |
| 1282 | Et | iBu | H | guanidinyl | H, iBu | CH₂COOH | H | OH | 15.3 | A | 939 | 1.49 | 0.0117 |
| 1283 | Et | iPr | H | guanidinyl | H, iBu | CH₂COOH | H | NH₂ | 3.5 | B | 931.3 | 1.68 | 0.0189 |
| 1284 | Et | nBu | H | guanidinyl | H, iBu | CH₂COOH | H | NH₂ | 7.2 | B | 1875.1 | 1.7 | 0.0036 |
| 1285 | Et | nBu | H | guanidinyl | H, iBu | CH₂COOH | H | OH | 10.4 | A | 939.3 | 1.51 | 0.0083 |
| 1286 | Et | iBu | H | guanidinyl | H, iBu | (CH₂)₂COOH | H | NH₂ | 20 | B | 1888.6 | 1.74 | 0.0392 |
| 1287 | Et | nBu | H | guanidinyl | H, iBu | (CH₂)₂COOH | H | NH₂ | 18 | B | 945.2 | 1.68 | 0.0427 |
| 1288 | Et | (CH₂)₄ | H | NH₂ | H, iBu | CH₂OH | H | OH | 18.2 | B | 911.2 | 1.46 | 0.0167 |
| | | NH₂ | | | | | | | | | | | |
| 1289 | Et | (CH₂)₄ | CH₂COOH | NH₂ | H, iBu | CH₂OH | H | OH | 23.8 | A | 940.2 | 1.39 | 0.0252 |
| | | NH₂ | | | | | | | | | | | |
| 1290 | Et | iBu | H | guanidinyl | Tic | CH₂OH | H | OH | 17.9 | A | 1894 | 1.58 | 0.0115 |
| 1291 | Et | iBu | H | guanidinyl | Me, | CH₂OH | H | OH | 19.6 | A | 1862.3 | 1.56 | 0.0051 |
| | | | | | nBu | | | | | | | | |
| 1292 | Et | iBu | H | guanidinyl | H, | CH₂OH | H | OH | 18.6 | A | 1898.2 | 1.51 | 0.0299 |
| | | | | | CH2- | | | | | | | | |
| | | | | | 4- | | | | | | | | |
| | | | | | Ohphenyl | | | | | | | | |
| 1293 | Et | iBu | H | guanidinyl | CH₂CONH₂ | CH₂OH | H | OH | 19.2 | B | 1848.8 | 1.54 | 0.0199 |
| 1294 | Et | iBu | H | guanidinyl | Me, | CH₂OH | H | NH₂ | 26.1 | A | 1862 | 1.67 | 0.0093 |
| | | | | | nBu | | | | | | | | |
| 1295 | Et | iBu | H | guanidinyl | CH₂CONH₂ | CH₂OH | H | NH₂ | 31.7 | A | 1848.1 | 1.52 | 0.0314 |
| 1296 | Et | iBu | H | guanidinyl | H, | CH₂OH | H | OH | 19.2 | A | 1864 | 1.48 | 0.0173 |
| | | | | | (CH₂)₄ | | | | | | | | |
| | | | | | NH₂ | | | | | | | | |
| 1297 | Et | iBu | H | guanidinyl | H, | CH₂OH | H | NH₂ | 13.7 | A | 1897.1 | 1.59 | 0.0357 |
| | | | | | CH₂-4- | | | | | | | | |
| | | | | | OH- | | | | | | | | |
| | | | | | phenyl | | | | | | | | |
| 1298 | Et | iBu | H | guanidinyl | H, | CH₂COOH | H | NH₂ | 26.5 | A | 1926.1 | 1.47 | 0.0361 |
| | | | | | CH₂-4- | | | | | | | | |
| | | | | | OH- | | | | | | | | |
| | | | | | phenyl | | | | | | | | |
| 1299 | Et | iBu | H | guanidinyl | H, | iPr | H | OH | 14.8 | A | 1909.9 | 1.58 | 0.0332 |
| | | | | | CH₂-4- | | | | | | | | |
| | | | | | OH- | | | | | | | | |
| | | | | | phenyl | | | | | | | | |
| 1300 | Et | nBu | CH₂CONH₂ | NHCONH₂ | H, iBu | CH₂COOH | H | OH | 4.7 | B | 968.3 | 1.65 | 0.0049 |
| 1301 | Et | nBu | CH₂CONH₂ | NHCONH₂ | Me, | CH₂COOH | H | OH | 27.5 | B | 975 | 1.63 | 0.0056 |
| | | | | | nBu | | | | | | | | |
| 1302 | Et | nPentyl | H | NHCONH₂ | H, iBu | CH₂OH | H | NH₂ | 15.4 | B | 932.1 | 1.78 | 0.0075 |
| 1303 | Et | nBu | H | NHCONH₂ | H, iBu | CH₂COOH | H | NH₂ | 22.3 | B | 939.2 | 1.66 | 0.0088 |
| 1304 | Et | nBu | CH₂CONH₂ | guanidinyl | Tic | CH₂OH | H | NH₂ | 54.1 | A | 976.2, | 1.74, | 0.0315 |
| | | | | | | | | | | | 976.2 | 1.78 | |
| 1305 | Et | nBu | CH₂CONH₂ | NHCONH₂ | Tic | CH₂COOH | H | NH₂ | 22.6 | B | 1979.1 | 1.79 | 0.0103 |
| 1306 | Et | nBu | CH₂CONH₂ | NHCONH₂ | Tic | CH₂OH | H | NH₂ | 2.6 | A | 1950.9 | 1.57 | 0.0252 |
| 1307 | Et | nBu | CH₂CONH₂ | guanidinyl | Tic | CH₂COOH | H | NH₂ | 24.8 | A | 990 | 1.5 | 0.0114 |
| 1308 | Et | nBu | CH₂CONH₂ | guanidinyl | Me, | CH₂COOH | H | OH | 18.4 | B | 1947.1 | 1.67 | 0.0095 |
| | | | | | nBu | | | | | | | | |
| 1309 | Et | iPr | H | guanidinyl | H, iBu | CH₂COOH | iBu | NH₂ | 13.8 | B | 959.2 | 1.73 | 0.0052 |
| 1310 | Et | iPr | H | guanidinyl | H, iBu | CH₂COOH | CH₂CONH₂ | NH₂ | 8.5 | B | 959.2 | 1.58 | 0.0034 |
| 1311 | Et | nBu | CH₂CONH₂ | guanidinyl | H, iBu | CH₂COOH | CH₂CONH₂ | NH₂ | 4 | A | 1988.9 | 1.56 | 0.0083 |
| 1312 | Et | iPr | H | guanidinyl | H, iBu | CH₂COOH | guanidinyl | NH₂ | 2.6 | B | 981.2 | 1.5 | 0.0077 |
| 1313 | Et | nBu | CH₂CONH₂ | guanidinyl | H, iBu | CH₂COOH | guanidinyl | NH₂ | 10.7 | A | 678.2 | 1.56 | 0.0031 |
| 1314 | Et | iPr | H | guanidinyl | H, iBu | CH₂COOH | CH₂OH | NH₂ | 7.4 | B | 946.1 | 1.58 | 0.0014 |
| 1315 | Et | nBu | CH₂CONH₂ | guanidinyl | H, iBu | CH₂COOH | CH₂OH | NH₂ | 17 | B | 981.1 | 1.6 | 0.0371 |
| 1316 | Et | iPr | H | NHCONH₂ | H, iBu | CH₂COOH | CH₂OH | NH₂ | 19.8 | A | 947.2 | 1.38 | 0.0106 |
| 1317 | Et | nBu | CH₂COOH | guanidinyl | H, iBu | CH₂COOH | CH₂OH | NH₂ | 15.1 | B | 982.1 | 1.69 | 0.0175 |
| 1318 | Et | nBu | CH₂COOH | guanidinyl | Me, | CH₂COOH | CH₂OH | NH₂ | 20.7 | B | 660 | 1.68 | 0.0119 |
| | | | | | nBu | | | | | | | | |
| 1319 | Et | nBu | CH₂COOH | NH₂ | H, iBu | CH₂COOH | CH₂OH | NH₂ | 8.3 | A | 1920.8 | 1.64 | 0.0137 |
| 1320 | Et | nBu | CH₂COOH | NH₂ | Me, | CH₂COOH | CH₂OH | NH₂ | 5 | A | 968.2 | 1.47 | 0.0213 |
| | | | | | nBu | | | | | | | | |
| 1321 | Et | nBu | CH₂CONH₂ | guanidinyl | H, iBu | CH₂COOH | CH₂- | NH₂ | 4.2 | A | 688.1 | 1.69 | 0.0159 |
| | | | | | | | indol- | | | | | | |
| | | | | | | | 3-yl | | | | | | |
| 1322 | Et | iPr | H | guanidinyl | H, iBu | CH₂COOH | CH₂-4- | NH₂ | 15.5 | A | 1967 | 1.58 | 0.0055 |
| | | | | | | | OH- | | | | | | |
| | | | | | | | phenyl | | | | | | |
| 1323 | Et | nBu | CH₂CONH₂ | guanidinyl | H, iBu | CH₂COOH | CH₂-4- | NH₂ | 5.3 | B | 1020.1 | 1.65 | 0.0135 |
| | | | | | | | OH- | | | | | | |
| | | | | | | | phenyl | | | | | | |

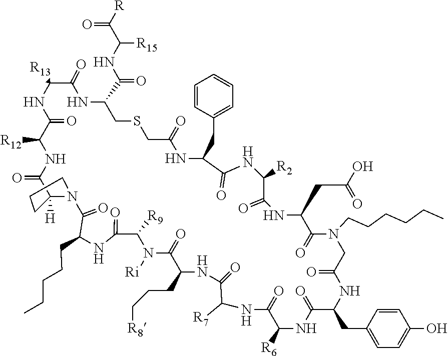

| | | | | | | | | | | | | | | HC |
|---|---|---|---|---|---|---|---|---|---|---|---|---|---|---|
| | | | | | | | | | | | | | | CBA |
| Ex. | | | | | | | | | | | Obs. | QC | | mVISTA |
| Num | | | | | Ri, | | | | | Yield | MS | Method | | IC50 |
| ber | R₂ | R₆ | R₇ | R_{8′} | R₉ | R₁₂ | R₁₃ | R₁₅ | R | (mg) | Ion | IDs | RT | (uM) |
| 1324 | CH₂- | iPr | H | guanidinyl | H, | iPr | CH₂COOH | H | NH₂ | 24.5 | 1846 | B | 1.75 | 0.0482 |
| | 2- | | | | iBu | | | | | | | | | |
| | naphthyl | | | | | | | | | | | | | |
| 1325 | CH₂- | npentyl | H | guanidinyl | H, | iPr | CH₂OH | H | NH₂ | 9.3 | 1795.2 | A | 2.09 | 0.0243 |
| | phenyl | | | | iBu | | | | | | | | | |
| 1326 | CH₂- | nBu | CH₂COOH | NH₂ | H, | iPr | CH₂COOH | CH₂COOH | NH₂ | 11.1 | 949.24 | B | 1.84 | 0.0175 |
| | 3- | | | | iBu | | | | | | | | | |
| | Me- | | | | | | | | | | | | | |
| | phenyl | | | | | | | | | | | | | |
| 1327 | CH₂- | nBu | CH₂COOH | guanidinyl | H, | iPr | CH₂COOH | CH₂COOH | NH₂ | 5.7 | 970.7 | A | 1.43 | 0.0139 |
| | 3- | | | | iBu | | | | | | | | | |
| | Me- | | | | | | | | | | | | | |
| | phenyl | | | | | | | | | | | | | |
| 1328 | CH₂- | iBu | CH₂CONH₂ | guanidinyl | H, | iPr | iPr | H | OH | 17.8 | 933.4 | B | 1.93 | 0.0020 |
| | 3- | | | | iBu | | | | | | | | | |
| | Me | | | | | | | | | | | | | |
| | phenyl | | | | | | | | | | | | | |
| 1329 | CH₂- | nBu | CH₂CONH₂ | guanidinyl | H, | iPr | CH₂COOH | H | OH | 16.3 | 941.5 | B | 1.76 | 0.0045 |
| | 3- | | | | iBu | | | | | | | | | |
| | Me- | | | | | | | | | | | | | |
| | phenyl | | | | | | | | | | | | | |
| 1330 | CH₂- | nBu | CH₂COOH | guanidinyl | H, | iPr | CH₂COOH | CH₂OH | NH₂ | 12.9 | 957.21 | B | 1.77 | 0.0168 |
| | 3- | | | | iBu | | | | | | | | | |
| | Me- | | | | | | | | | | | | | |
| | phenyl | | | | | | | | | | | | | |
| 1331 | CH₂- | nBu | CH₂COOH | NH₂ | H, | iPr | CH₂COOH | CH₂OH | NH₂ | 5.5 | 1869.7 | B | 1.84 | 0.0143 |
| | 3- | | | | iBu | | | | | | | | | |
| | Me- | | | | | | | | | | | | | |
| | phenyl | | | | | | | | | | | | | |
| 1332 | CH₂- | nBu | CH₂CONH₂ | guanidinyl | H, | iPr | CH₂COOH | H | OH | 9.5 | 968.5 | B | 1.84 | 0.0121 |
| | 3- | | | | iBu | | | | | | | | | |
| | CF₃- | | | | | | | | | | | | | |
| | phenyl | | | | | | | | | | | | | |
| 1333 | CH₂- | iBu | CH₂CONH₂ | guanidinyl | H, | iPr | iPr | H | OH | 18.7 | 960.4 | B | 1.98 | 0.0276 |
| | 3- | | | | iBu | | | | | | | | | |
| | CF₃- | | | | | | | | | | | | | |
| | phenyl | | | | | | | | | | | | | |
| 1334 | CH₂- | iBu | CH₂CONH₂ | guanidinyl | H, | iPr | iPr | H | OH | 22.6 | 944.6 | B | 1.9 | 0.0053 |
| | 3- | | | | iBu | | | | | | | | | |
| | Br- | | | | | | | | | | | | | |
| | phenyl | | | | | | | | | | | | | |
| 1335 | CH₂- | nBu | CH₂CONH₂ | guanidinyl | H, | iPr | CH₂COOH | H | OH | 26.2 | 952.5 | B | 1.75 | 0.0022 |
| | 3- | | | | iBu | | | | | | | | | |
| | Br- | | | | | | | | | | | | | |
| | phenyl | | | | | | | | | | | | | |
| 1336 | CH₂- | nBu | CH₂CONH₂ | guanidinyl | H, | iPr | CH₂COOH | CH₂OH | OH | 7.2 | 967.3 | B | 1.72 | 0.0207 |
| | 3- | | | | iBu | | | | | | | | | |
| | Br- | | | | | | | | | | | | | |
| | phenyl | | | | | | | | | | | | | |
| 1337 | CH₂- | iBu | CH₂CONH₂ | guanidinyl | H, | iPr | iPr | CH₂OH | OH | 13.8 | 959.1 | A | 1.67 | 0.0182 |
| | 3- | | | | iBu | | | | | | | | | |
| | Br- | | | | | | | | | | | | | |
| | phenyl | | | | | | | | | | | | | |
| 1338 | CH₂- | iBu | CH₂CONH₂ | guanidinyl | Me, | tBu | CH₂OH | H | NH₂ | 38 | 1922.2 | A | 1.85, | 0.0346 |
| | 2- | | | | nBu | | | | | | | | 1.89 | |
| | O- | | | | | | | | | | | | | |
| | allyl- | | | | | | | | | | | | | |
| | phenyl | | | | | | | | | | | | | |
| 1339 | CH₂- | iBu | CH₂CONH₂ | guanidinyl | Me, | tBu | CH₂OH | H | NH₂ | 50.2 | 951 | B | 1.83 | 0.0316 |
| | 3- | | | | nBu | | | | | | | | | |
| | Cl- | | | | | | | | | | | | | |
| | phenyl | | | | | | | | | | | | | |
| 1340 | CH₂- | iBu | CH₂CONH₂ | guanidinyl | Me, | tBu | CH₂OH | H | NH₂ | 37.8 | 1891.2 | A | 1.71 | 0.0368 |
| | 3- | | | | nBu | | | | | | | | | |
| | CN- | | | | | | | | | | | | | |
| | phenyl | | | | | | | | | | | | | |
| 1341 | CH₂- | iBu | CH₂CONH₂ | guanidinyl | Me, | tBu | CH₂OH | H | NH₂ | 40.4 | 1884.3 | A | 1.76 | 0.0191 |
| | 3-F- | | | | nBu | | | | | | | | | |
| | phenyl | | | | | | | | | | | | | |
| 1342 | CH₂- | nBu | CH₂COOH | guanidinyl | Me, | iPr | CH₂COOH | CH₂COOH | NH₂ | 14.1 | 1953.3 | B | 1.84 | 0.0125 |
| | 3- | | | | nBu | | | | | | | | | |
| | Me- | | | | | | | | | | | | | |
| | phenyl | | | | | | | | | | | | | |
| 1343 | CH₂- | nBu | CH₂COOH | NH₂ | Me, | iPr | CH₂COOH | CH₂COOH | NH₂ | 22.7 | 1911 | A | 1.5 | 0.0178 |
| | 3- | | | | nBu | | | | | | | | | |
| | Me- | | | | | | | | | | | | | |
| | phenyl | | | | | | | | | | | | | |
| 1344 | CH₂- | npentyl | CH₂CONH₂ | guanidinyl | Me, | iPr | iPr | H | OH | 28.6 | 947.4 | B | 1.96 | 0.0110 |
| | 3- | | | | nBu | | | | | | | | | |
| | Me- | | | | | | | | | | | | | |
| | phenyl | | | | | | | | | | | | | |
| 1345 | CH₂- | nBu | CH₂CONH₂ | guanidinyl | Me, | iPr | CH₂COOH | H | OH | 16.1 | 948 | A | 1.48 | 0.0127 |
| | 3- | | | | nBu | | | | | | | | | |
| | Me- | | | | | | | | | | | | | |
| | phenyl | | | | | | | | | | | | | |
| 1346 | CH₂- | npentyl | CH₂CONH₂ | guanidinyl | Me, | iPr | iPr | CH₂OH | OH | 22.3 | 961.9 | B | 1.98 | 0.0061 |
| | 3- | | | | nBu | | | | | | | | | |
| | Me- | | | | | | | | | | | | | |
| | phenyl | | | | | | | | | | | | | |
| 1347 | CH₂- | nBu | CH₂COOH | guanidinyl | Me, | iPr | CH₂COOH | CH₂OH | NH₂ | 12.8 | 964.1 | B | 1.78 | 0.0106 |
| | 3- | | | | nBu | | | | | | | | | |
| | Me- | | | | | | | | | | | | | |
| | phenyl | | | | | | | | | | | | | |
| 1348 | CH₂- | nBu | CH₂COOH | NH₂ | Me, | iPr | CH₂COOH | CH₂OH | NH₂ | 9.4 | 1882.8 | B | 1.83 | 0.0232 |
| | 3- | | | | nBu | | | | | | | | | |
| | Me- | | | | | | | | | | | | | |
| | phenyl | | | | | | | | | | | | | |
| 1349 | CH₂- | iBu | CH₂CONH₂ | guanidinyl | Me, | tBu | CH₂OH | H | NH₂ | 43.3 | 1880.1 | B | 1.83 | 0.0436 |
| | 4- | | | | nBu | | | | | | | | | |
| | Me- | | | | | | | | | | | | | |
| | phenyl | | | | | | | | | | | | | |
| 1350 | CH₂- | n- | CH₂CONH₂ | guanidinyl | Me, | iPr | CH₂COOH | H | OH | 19.4 | 966.2 | B | 1.85 | 0.0121 |
| | 3- | pentyl | | | nBu | | | | | | | | | |
| | Br- | | | | | | | | | | | | | |
| | phenyl | | | | | | | | | | | | | |
| 1351 | CH₂- | n- | CH₂CONH₂ | guanidinyl | Me, | iPr | CH₂COOH | CH₂OH | OH | 14.9 | 981.3 | B | 1.76 | 0.0147 |
| | 3- | pentyl | | | nBu | | | | | | | | | |
| | Br- | | | | | | | | | | | | | |
| | phenyl | | | | | | | | | | | | | |

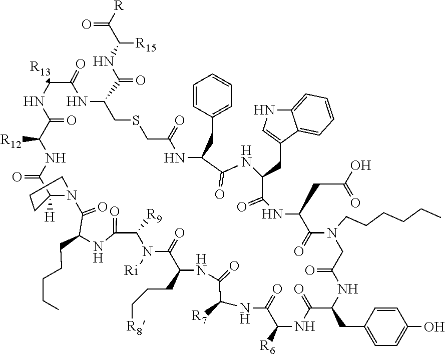

| | | | | | | | | | | | | | HC |
|---|---|---|---|---|---|---|---|---|---|---|---|---|---|
| | | | | | | | | | | | | | CBA |
| | | | | | | | | | | QC | Obs. | | mVISTA |
| Example | | | | | | | | | Yield | Method | MS | | IC50 |
| Number | R₆ | R₇ | R_{8′} | Rᵢ, R₉ | R₁₂ | R₁₃ | R₁₅ | R | (mg) | IDs | Ion | RT | (uM) |
| 1352 | nBu | CH₂COOH | guanidinyl | H, iBu | iPr | CH₂COOH | CH₂COOH | NH₂ | 6.6 | B | 983 | 1.59 | 0.0123 |
| 1353 | nBu | CH₂COOH | NH2 | H, iBu | iPr | CH₂COOH | CH₂COOH | NH₂ | 10.5 | A | 1923.2 | 1.52 | 0.0113 |
| 1354 | npentyl | CH₂CONH₃ | guanidinyl | H, iBu | iPr | CH₂OH | H | NH₂ | 12.3 | A | 1892.2 | 1.74 | 0.0203 |
| 1355 | nBu | CH₂COOH | NH2 | H, iBu | iPr | CH₂OH | H | OH | 27.4 | A | 918.9 | 1.5 | 0.0338 |
| 1356 | nBu | CH₂COOH | guanidinyl | H, iBu | iPr | CH₂OH | H | OH | 25.3 | A | 940.1 | 1.46 | 0.0287 |
| 1357 | npentyl | CH₂CONH₃ | guanidinyl | H, iBu | iPr | CH₂OH | H | OH | 17.8 | B | 1891.9 | 1.9 | 0.0316 |
| 1358 | iBu | H | guanidinyl | H, iBu | iPr | CH₂OH | H | NH₂ | 9.5 | B | 1819.9 | 2.01 | 0.0195 |
| 1359 | iBu | CH₂COOH | NH2 | H, iBu | iPr | CH₂OH | H | NH₂ | 5.6 | B | 1836 | 1.86 | 0.0171 |
| 1360 | nBu | CH₂CONH₃ | guanidinyl | H, iBu | iPr | (CH₂)₃- | H | OH | 6.1 | B | 650.1 | 1.64 | 0.0284 |
| | | | | | | guanidinyl | | | | | | | |
| 1361 | nBu | CH₂CONH₃ | guanidinyl | H, iBu | iPr | CH₂COOH | H | OH | 19.3 | A | 1906.1 | 1.48 | 0.0052 |
| 1362 | nBu | CH₂CONH₃ | guanidinyl | H, iBu | iPr | NMe- | H | OH | 35.3 | B | 1863.2 | 1.81 | 0.0379 |
| | | | | | | Gly | | | | | | | |
| 1363 | nBu | CH₂CONH₃ | NHCONH2 | H, iBu | iPr | CH₂COOH | H | OH | 28.7 | B | 954.2 | 1.69 | 0.0040 |
| 1364 | nBu | CH₂CONH₃ | guanidinyl | H, iBu | tBu | CH₂OH | H | NH₂ | 22.9 | B | 1890.8 | 1.87 | 0.0440 |
| 1365 | nBu | CH₂COOH | NH2 | H, iBu | iPr | CH₂COOH | CH₂OH | NH₂ | 9.1 | A | 1895.2 | 1.61 | 0.0077 |
| 1366 | nBu | CH₂COOH | guanidinyl | H, iBu | iPr | CH₂COOH | CH₂OH | NH₂ | 8 | A | 968.9 | 1.41 | 0.0116 |
| 1367 | nBu | CH₂CONH₃ | guanidinyl | Me, | iPr | CH₂COOH | H | OH | 8.8 | B | 1920.1 | 1.49 | 0.0111 |
| | | | | CHMe | | | | | | | | | |
| | | | | Et | | | | | | | | | |
| 1368 | nBu | CH₂CONH₃ | guanidinyl | Me, | iPr | CH₂COOH | H | OH | 26.2 | A | 1922 | 1.16 | 0.0370 |
| | | | | CH₂COOH | | | | | | | | | |
| 1369 | nBu | CH₂CONH₃ | guanidinyl | Me, | iPr | CH₂COOH | H | OH | 26.6 | B | 1921.1 | 1.54, | 0.0035 |
| | | | | iBu | | | | | | | | 1.58 | |
| 1370 | nBu | CH₂CONH₃ | guanidinyl | Me, | iPr | CH₂COOH | CH₂OH | OH | 6.1 | A | 975.9 | 1.36 | 0.0187 |
| | | | | iBu | | | | | | | | | |
| 1371 | nBu | CH₂CONH₃ | guanidinyl | Me, | tBu | CH₂COOH | CH₂COOH | NH₂ | 11.8 | A | 1991 | 1.52 | 0.0366 |
| | | | | nBu | | | | | | | | | |
| 1372 | nBu | CH₂COOH | NH2 | Me, | tBu | CH₂COOH | CH₂COOH | NH₂ | 24.5 | B | 1950.2 | 1.76 | 0.0164 |
| | | | | nBu | | | | | | | | | |
| 1373 | nBu | CH₂CONH₃ | NH2 | Me, | tBu | CH₂COOH | CH₂COOH | NH₂ | 16.2 | B | 1948.9 | 1.73 | 0.0148 |
| | | | | nBu | | | | | | | | | |
| 1374 | nBu | CH₂CONH₃ | NH2 | Me, | tBu | CH₂OH | CH₂COOH | NH₂ | 12.2 | B | 1921.3 | 1.84 | 0.0249 |
| | | | | nBu | | | | | | | | | |
| 1375 | nBu | CH₂COOH | guanidinyl | Me, | tBu | CH₂OH | CH₂COOH | NH₂ | 13.5 | B | 1964.2 | 1.85 | 0.0425 |
| | | | | nBu | | | | | | | | | |
| 1376 | nBu | CH₂COOH | NH2 | Me, | tBu | CH₂OH | CH₂COOH | NH₂ | 16.4 | B | 1923 | 1.78 | 0.0366 |
| | | | | nBu | | | | | | | | | |
| 1377 | nBu | CH₂COOH | guanidinyl | Me, | tBu | CH₂COOH | CH₂COOH | NH₂ | 17.4 | B | 1992 | 1.75 | 0.0222 |
| | | | | nBu | | | | | | | | | |
| 1378 | nBu | CH₂CONH₃ | guanidinyl | Me, | tBu | CH₂OH | CH₂COOH | NH₂ | 18.4 | B | 1964 | 1.78 | 0.0413 |
| | | | | nBu | | | | | | | | | |
| 1379 | nBu | CH₂CONH₃ | guanidinyl | Me, | iPr | CH₂COOH | H | OH | 39.3 | A | 1920 | 1.42 | 0.0059 |
| | | | | nBu | | | | | | | | | |
| 1380 | nBu | CH₂CONH₃ | guanidinyl | Me, | iPr | (CH₂)₃- | H | OH | 20.7 | B | 1961.2 | 1.69 | 0.0121 |
| | | | | nBu | | guanidinyl | | | | | | | |
| 1381 | nBu | CH₂CONH₃ | guanidinyl | Me, | iPr | CH₂CONH₃ | H | OH | 27.6 | A | 1919.1 | 1.49 | 0.0203 |
| | | | | nBu | | | | | | | | | |
| 1382 | nBu | CH₂CONH₃ | guanidinyl | Me, | iPr | CH₂OH | H | OH | 29.1 | A | 1893.1 | 1.66 | 0.0172 |
| | | | | nBu | | | | | | | | | |
| 1383 | nBu | CH₂CONH₃ | guanidinyl | Me, | iPr | iPr | H | OH | 29.6 | B | 1904 | 1.71 | 0.0169 |
| | | | | nBu | | | | | | | | | |
| 1384 | npentyl | CH₂CONH₃ | guanidinyl | Me, | iPr | CH₂OH | H | OH | 40.2 | A | 954.1 | 1.54 | 0.0109 |
| | | | | nBu | | | | | | | | | |
| 1385 | npentyl | CH₂CONH₃ | guanidinyl | Me, | iPr | iPr | H | OH | 28.7 | A | 960.1 | 1.62 | 0.0105 |
| | | | | nBu | | | | | | | | | |
| 1386 | npentyl | CH₂CONH₃ | guanidinyl | Me, | iPr | CH₂COOH | H | OH | 38 | B | 968.2 | 1.71 | 0.0088 |
| | | | | nBu | | | | | | | | | |
| 1387 | nBu | CH₂CONH₃ | NHCONH2 | Me, | iPr | CH₂COOH | H | OH | 10.9 | A | 961.2 | 1.33 | 0.0053 |
| | | | | nBu | | | | | | | | | |
| 1388 | nBu | CH₂CONH₃ | NHCONH2 | Me, | CH | CH₂OH | H | NH₂ | 12.7 | A | 954 | 1.53 | 0.0164 |
| | | | | nBu | MeEt | | | | | | | | |
| 1389 | nBu | CH₂CONH₃ | guanidinyl | Me, | cPr | CH₂OH | H | NH₂ | 31.7 | B | 945 | 1.71 | 0.0344 |
| | | | | nBu | | | | | | | | | |
| 1390 | nBu | CH₂CONH₃ | NHCONH2 | Me, | tBu | CH₂OH | H | NH₂ | 13.7 | A | 954 | 1.53 | 0.0252 |
| | | | | nBu | | | | | | | | | |
| 1391 | nBu | CH₂CONH₃ | guanidinyl | Me, | tBu | CH₂OH | H | NH₂ | 15.8 | B | 636.2 | 1.65 | 0.0363 |
| | | | | nBu | | | | | | | | | |
| 1392 | nBu | CH₂COOH | guanidinyl | Me, | tBu | CH₂COOH | H | NH₂ | 17.5 | A | 1934 | 1.52 | 0.0383 |
| | | | | nBu | | | | | | | | | |
| 1393 | nBu | CH₂COOH | NH2 | Me, | tBu | CH₂COOH | H | NH₂ | 23.2 | A | 1891.7 | 1.55 | 0.0172 |
| | | | | nBu | | | | | | | | | |
| 1394 | nBu | CH₂CONH₃ | NH2 | Me, | tBu | CH₂COOH | H | NH₂ | 21.6 | B | 1892.2 | 1.75 | 0.0348 |
| | | | | nBu | | | | | | | | | |
| 1395 | nBu | CH₂COOH | guanidinyl | Me, | tBu | CH₂OH | H | NH₂ | 23.6 | A | 1906.3 | 1.71 | 0.0486 |
| | | | | nBu | | | | | | | | | |
| 1396 | nBu | CH₂CONH₃ | guanidinyl | Me, | tBu | CH₂COOH | H | NH₂ | 16.8 | B | 1933.3 | 1.81 | 0.0268 |
| | | | | nBu | | | | | | | | | |
| 1397 | nBu | CH₂COOH | NH2 | Me, | tBu | CH₂COOH | CH₂CONH₃ | OH | 13.3 | B | 1951.2 | 1.75 | 0.0360 |
| | | | | nBu | | | | | | | | | |
| 1398 | nBu | CH₂CONH₃ | guanidinyl | Me, | tBu | CH₂COOH | CH₂CONH₃ | OH | 19.1 | B | 1992 | 1.73 | 0.0439 |
| | | | | nBu | | | | | | | | | |
| 1399 | nBu | CH₂COOH | guanidinyl | Me, | tBu | CH₂OH | CH₂CONH₃ | OH | 17.5 | A | 1963.7 | 1.52 | 0.0273 |
| | | | | nBu | | | | | | | | | |
| 1400 | nBu | CH₂CONH₃ | NH2 | Me, | tBu | CH₂OH | CH₂CONH₃ | OH | 23.5 | B | 1921.3 | 1.77 | 0.0141 |
| | | | | nBu | | | | | | | | | |
| 1401 | nBu | CH₂COOH | NH2 | Me, | tBu | CH₂OH | CH₂CONH₃ | OH | 13.1 | A | 1922 | 1.54 | 0.0202 |
| | | | | nBu | | | | | | | | | |
| 1402 | nBu | CH₂CONH₃ | guanidinyl | Me, | tBu | CH₂OH | CH₂CONH₃ | OH | 19.1 | B | 1963.2 | 1.76 | 0.0338 |
| | | | | nBu | | | | | | | | | |
| 1403 | nBu | CH₂COOH | guanidinyl | Me, | tBu | CH₂COOH | CH₂CONH₃ | OH | 20.8 | B | 1993.2 | 1.8 | 0.0320 |
| | | | | nBu | | | | | | | | | |
| 1404 | nBu | CH₂CONH₃ | NH2 | Me, | tBu | CH₂COOH | CH₂CONH₃ | OH | 14.3 | B | 1949.2 | 1.69 | 0.0160 |
| | | | | nBu | | | | | | | | | |
| 1405 | n- | CH₂CONH₃ | guanidinyl | Me, | iPr | CH₂COOH | CH₂OH | NH₂ | 38.3 | B | 982.2 | 1.73 | 0.0050 |
| | pentyl | | | nBu | | | | | | | | | |
| 1406 | nBu | CH₂CONH₃ | guanidinyl | Me, | iPr | CH₂COOH | CH₂OH | OH | 12 | B | 976 | 1.73 | 0.0064 |
| | | | | nBu | | | | | | | | | |
| 1407 | nBu | CH₂COOH | guanidinyl | Me, | iPr | CH₂COOH | CH₂OH | NH₂ | 38.4 | A | 976.1 | 1.33 | 0.0345 |
| | | | | nBu | | | | | | | | | |
| 1408 | nBu | CH₂CONH₃ | guanidinyl | Me, | iPr | CH₂COOH | H | OH | 25.4 | B | 1894 | 1.4 | 0.0208 |
| | | | | CH2OH | | | | | | | | | |
| 1409 | nBu | CH₂CONH₃ | guanidinyl | Me, | iPr | CH₂COOH | H | OH | 9.9 | B | 1906 | 1.45 | 0.0127 |
| | | | | iPr | | | | | | | | | |
| 1410 | nBu | CH₂CONH₃ | guanidinyl | Me, | iPr | CH₂COOH | H | OH | 21.6 | A | 1992.9 | 1.66 | 0.0053 |
| | | | | CH2- | | | | | | | | | |
| | | | | indol- | | | | | | | | | |
| | | | | 3-yl | | | | | | | | | |
| 1411 | nBu | CH₂CONH₃ | guanidinyl | Me, | iPr | CH₂COOH | H | OH | 19.2 | B | 1970.1 | 1.45 | 0.0186 |
| | | | | CH2- | | | | | | | | | |
| | | | | 4- | | | | | | | | | |
| | | | | OHphenyl | | | | | | | | | |
| 1412 | nBu | CH₂CONH₃ | NHCONH2 | Tic | iPr | CH₂COOH | H | NH₂ | 12.2 | A | 977.1 | 1.42 | 0.0131 |
| 1413 | nBu | CH₂CONH₃ | NHCONH2 | Tic | iPr | CH₂OH | H | NH₂ | 18.7 | B | 963.2 | 1.77 | 0.0189 |
| 1414 | nBu | CH₂CONH₃ | guanidinyl | Tic | iPr | CH₂COOH | H | NH₂ | 25.4 | A | 976.2 | 1.62 | 0.0123 |
| 1415 | nBu | CH₂CONH₃ | guanidinyl | Tic | iPr | CH₂OH | H | NH₂ | 20.3 | B | 642.2 | 1.65 | 0.0181 |
| 1416 | iBu | H | guanidinyl | H, | iPr | CH₂COOH | H | NH₂ | 30.5 | B | 1898.2 | 1.6 | 0.0517 |
| | | | | CH2- | | | | | | | | | |
| | | | | 4- | | | | | | | | | |
| | | | | OHphenyl | | | | | | | | | |
| 1417 | iBu | H | guanidinyl | H, | iPr | CH₂COOH | H | OH | 29.4 | A | 1898.9 | 1.37 | 0.0249 |
| | | | | CH2- | | | | | | | | | |
| | | | | 4- | | | | | | | | | |
| | | | | OHphenyl | | | | | | | | | |

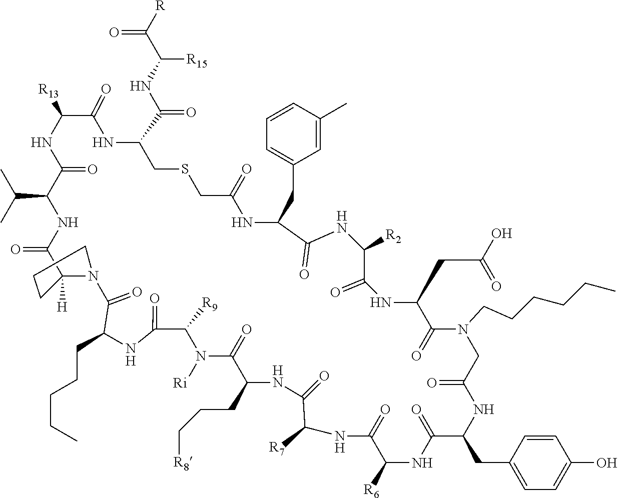

| | | | | | | | | | | | | | HC |
|---|---|---|---|---|---|---|---|---|---|---|---|---|---|
| | | | | | | | | | | | | | CBA |
| | | | | | | | | | | QC | Obs. | | mVISTA |
| Ex. | | | | | Rᵢ, | | | | Yield | Method | MS | | IC50 |
| Number. | R₂ | R₆ | R₇ | R_{8′} | R₉ | R₁₃ | R₁₅ | R | (mg) | IDs | Ion | RT | (uM) |
| 1418 | CH₂- | nBu | CH₂COOH | NH₂ | H, | CH₂COOH | CH₂COOH | NH₂ | 10.9 | A | 1937.1 | 1.56 | 0.0076 |
| | indol- | | | | iBu | | | | | | | | |
| | 3- | | | | | | | | | | | | |
| | yl | | | | | | | | | | | | |
| 1419 | CH₂- | nBu | CH₂COOH | guanidinyl | H, | CH₂COOH | CH₂COOH | NH₂ | 6.7 | A | 1979.3 | 1.53 | 0.0081 |
| | indol- | | | | iBu | | | | | | | | |
| | 3- | | | | | | | | | | | | |
| | yl | | | | | | | | | | | | |
| 1420 | CH₂- | nBu | CH₂CONH₂ | guanidinyl | H, | CH₂COOH | H | OH | 9.7 | A | 961.3 | 1.5 | 0.0020 |
| | indol- | | | | iBu | | | | | | | | |
| | 3- | | | | | | | | | | | | |
| | yl | | | | | | | | | | | | |
| 1421 | CH₂- | nBu | H | guanidinyl | H, | CH₂COOH | H | OH | 19.8 | A | 932.1 | 1.56 | 0.0031 |
| | indol- | | | | iBu | | | | | | | | |
| | 3- | | | | | | | | | | | | |
| | yl | | | | | | | | | | | | |
| 1422 | CH₂- | iBu | CH₂CONH₂ | guanidinyl | H, | iPr | H | OH | 23.6 | A | 1905.1 | 1.88 | 0.0042 |
| | indol- | | | | iBu | | | | | | | | |
| | 3- | | | | | | | | | | | | |
| | yl | | | | | | | | | | | | |
| 1423 | CH₂- | nBu | CH₂CONH₂ | NHCONH2 | H, | CH₂COOH | H | OH | 16.3 | B | 641.1 | 1.82 | 0.0167 |
| | indol- | | | | iBu | | | | | | | | |
| | 3- | | | | | | | | | | | | |
| | yl | | | | | | | | | | | | |
| 1424 | CH₂- | nBu | CH₂CONH₂ | guanidinyl | H, | CH₂COOH | CH₂OH | OH | 13.5 | A | 976.3 | 1.48 | 0.0119 |
| | indol- | | | | iBu | | | | | | | | |
| | 3- | | | | | | | | | | | | |
| | yl | | | | | | | | | | | | |
| 1425 | CH₂- | nBu | H | guanidinyl | H, | CH₂COOH | CH₂OH | OH | 11.4 | B | 947.4 | 1.7 | 0.0134 |
| | indol- | | | | iBu | | | | | | | | |
| | 3- | | | | | | | | | | | | |
| | yl | | | | | | | | | | | | |
| 1426 | CH₂- | iBu | CH₂CONH₂ | guanidinyl | H, | iPr | CH₂OH | OH | 19.3 | B | 967.9 | 1.99 | 0.0041 |
| | indol- | | | | iBu | | | | | | | | |
| | 3- | | | | | | | | | | | | |
| | yl | | | | | | | | | | | | |
| 1427 | CH₂- | nBu | CH₂COOH | NH2 | H, | CH₂COOH | CH₂OH | NH₂ | 9.6 | B | 955.1 | 1.73 | 0.0094 |
| | indol- | | | | iBu | | | | | | | | |
| | 3- | | | | | | | | | | | | |
| | yl | | | | | | | | | | | | |
| 1428 | CH₂- | nBu | CH₂COOH | guanidinyl | H, | CH₂COOH | CH₂OH | NH₂ | 11.8 | A | 976 | 1.52 | 0.0136 |
| | indol- | | | | iBu | | | | | | | | |
| | 3- | | | | | | | | | | | | |
| | yl | | | | | | | | | | | | |
| 1429 | CH₂- | nBu | CH₂COOH | guanidinyl | H, | CH₂COOH | v | NH₂ | 10.6 | A | 1955 | 1.43 | 0.0105 |
| | 4- | | | | iBu | | | | | | | | |
| | OH- | | | | | | | | | | | | |
| | phenyl | | | | | | | | | | | | |
| 1430 | CH₂- | nBu | CH₂COOH | NH2 | H, | CH₂COOH | CH₂COOH | NH₂ | 21.7 | A | 1912.6 | 1.42 | 0.0062 |
| | 4- | | | | iBu | | | | | | | | |
| | OH- | | | | | | | | | | | | |
| | phenyl | | | | | | | | | | | | |
| 1431 | CH2- | nBu | H | guanidinyl | H, | CH₂COOH | H | OH | 20.3 | A | 1840.11 | 1.58 | 0.0025 |
| | 4- | | | | iBu | | | | | | | | |
| | OH- | | | | | | | | | | | | |
| | phenyl | | | | | | | | | | | | |
| 1432 | CH2- | iBu | CH₂CONH₂ | guanidinyl | H, | iPr | H | OH | 18.4 | B | 1881.3 | 1.74 | 0.0030 |
| | 4- | | | | iBu | | | | | | | | |
| | OH- | | | | | | | | | | | | |
| | phenyl | | | | | | | | | | | | |
| 1433 | CH₂- | nBu | CH₂CONH₂ | guanidinyl | H, | CH₂COOH | H | OH | 7.9 | B | 947.5 | 1.7 | 0.0342 |
| | 4- | | | | iBu | | | | | | | | |
| | OH- | | | | | | | | | | | | |
| | phenyl | | | | | | | | | | | | |
| 1434 | CH₂- | iBu | CH₂CONH₂ | guanidinyl | H, | iPr | CH₂OH | OH | 6.6 | A | 956 | 1.58 | 0.0204 |
| | 4- | | | | iBu | | | | | | | | |
| | OH- | | | | | | | | | | | | |
| | phenyl | | | | | | | | | | | | |
| 1435 | CH₂- | nBu | H | guanidinyl | H, | CH₂COOH | CH₂OH | OH | 4.2 | B | 936.1 | 1.7 | 0.0094 |
| | 4- | | | | iBu | | | | | | | | |
| | OH- | | | | | | | | | | | | |
| | phenyl | | | | | | | | | | | | |
| 1436 | CH₂- | nBu | CH₂COOH | NH2 | H, | CH₂COOH | CH₂OH | NH₂ | 5.3 | A | 943.2 | 1.37 | 0.0106 |
| | 4- | | | | iBu | | | | | | | | |
| | OH- | | | | | | | | | | | | |
| | phenyl | | | | | | | | | | | | |
| 1437 | CH₂- | nBu | CH₂COOH | guanidinyl | H, | CH₂COOH | CH₂OH | NH₂ | 17.9 | A | 963.9 | 1.37 | 0.0182 |
| | 4- | | | | iBu | | | | | | | | |
| | OH- | | | | | | | | | | | | |
| | phenyl | | | | | | | | | | | | |
| 1438 | CH₂- | nBu | CH₂COOH | NHCONH2 | Me, | CH₂COOH | CH₂COOH | NH₂ | 15.9 | A | 997.2 | 1.33 | 0.0224 |
| | indol- | | | | nBu | | | | | | | | |
| | 3- | | | | | | | | | | | | |
| | yl | | | | | | | | | | | | |
| 1439 | CH₂- | nBu | CH₂COOH | NH₂ | Me, | CH₂COOH | CH₂COOH | NH₂ | 12 | A | 1950.2 | 1.51 | 0.0093 |
| | indol- | | | | nBu | | | | | | | | |
| | 3- | | | | | | | | | | | | |
| | yl | | | | | | | | | | | | |
| 1440 | CH₂- | nBu | CH₂COOH | guanidinyl | Me, | CH₂COOH | CH₂COOH | NH₂ | 15.9 | B | 1992.1 | 1.83 | 0.0148 |
| | indol- | | | | nBu | | | | | | | | |
| | 3- | | | | | | | | | | | | |
| | yl | | | | | | | | | | | | |
| 1441 | CH₂- | n- | CH₂CONH₂ | guanidinyl | Me, | CH₂COOH | H | OH | 40.8 | A | 975.2 | 1.5 | 0.0102 |
| | indol- | Pentyl | | | nBu | | | | | | | | |
| | 3- | | | | | | | | | | | | |
| | yl | | | | | | | | | | | | |
| 1442 | CH₂- | nBu | CH₂CONH₂ | guanidinyl | Me, | CH₂COOH | H | OH | 13.7 | B | 968 | 1.76 | 0.0277 |
| | indol- | | | | nBu | | | | | | | | |
| | 3- | | | | | | | | | | | | |
| | yl | | | | | | | | | | | | |
| 1443 | CH₂- | nBu | CH₂CONH₂ | NHCONH₂ | Me, | CH₂COOH | H | OH | 8.9 | B | 968.1 | 1.79 | 0.0212 |
| | indol- | | | | nBu | | | | | | | | |
| | 3- | | | | | | | | | | | | |
| | yl | | | | | | | | | | | | |
| 1444 | CH₂- | n- | CH₂CONH₂ | guanidinyl | Me, | CH₂COOH | CH₂OH | OH | 12 | B | 990.3 | 1.72 | 0.0133 |
| | indol- | Pentyl | | | nBu | | | | | | | | |
| | 3- | | | | | | | | | | | | |
| | yl | | | | | | | | | | | | |
| 1445 | CH₂- | nBu | CH₂COOH | guanidinyl | Me, | CH₂COOH | CH₂OH | NH₂ | 9.5 | B | 983.2 | 1.72 | 0.0099 |
| | indol- | | | | nBu | | | | | | | | |
| | 3- | | | | | | | | | | | | |
| | yl | | | | | | | | | | | | |
| 1446 | CH₂- | nBu | CH₂COOH | NH₂ | Me, | CH₂COOH | CH₂OH | NH₂ | 9.8 | A | 1921.7 | 1.64 | 0.0091 |
| | indol- | | | | nBu | | | | | | | | |
| | 3- | | | | | | | | | | | | |
| | yl | | | | | | | | | | | | |
| 1447 | CH₂- | nBu | CH₂COOH | NHCONH₂ | Me, | CH₂COOH | CH₂COOH | NH₂ | 9.4 | B | 986.1 | 1.64 | 0.0153 |
| | 4- | | | | nBu | | | | | | | | |
| | OH- | | | | | | | | | | | | |
| | phenyl | | | | | | | | | | | | |
| 1448 | CH₂- | nBu | CH₂COOH | guanidinyl | Me, | CH₂COOH | CH₂COOH | NH₂ | 8.6 | A | 1969.2 | 1.41 | 0.0142 |
| | 4- | | | | nBu | | | | | | | | |
| | OH- | | | | | | | | | | | | |
| | phenyl | | | | | | | | | | | | |
| 1449 | CH₂- | nBu | CH₂COOH | NH₂ | Me, | CH₂COOH | CH₂COOH | NH₂ | 17 | B | 1927 | 1.76 | 0.0108 |
| | 4- | | | | nBu | | | | | | | | |
| | OH- | | | | | | | | | | | | |
| | phenyl | | | | | | | | | | | | |
| 1450 | CH₂- | nBu | CH₂CONH₂ | guanidinyl | Me, | CH₂COOH | H | OH | 11.7 | A | 1912.2 | 1.51 | 0.0070 |
| | 4- | | | | nBu | | | | | | | | |
| | OH- | | | | | | | | | | | | |
| | phenyl | | | | | | | | | | | | |
| 1451 | CH₂- | nBu | CH₂COOH | guanidinyl | Me, | CH₂COOH | CH₂OH | NH₂ | 11.1 | A | 1940.7 | 1.52 | 0.0086 |
| | 4- | | | | nBu | | | | | | | | |
| | OH- | | | | | | | | | | | | |
| | phenyl | | | | | | | | | | | | |

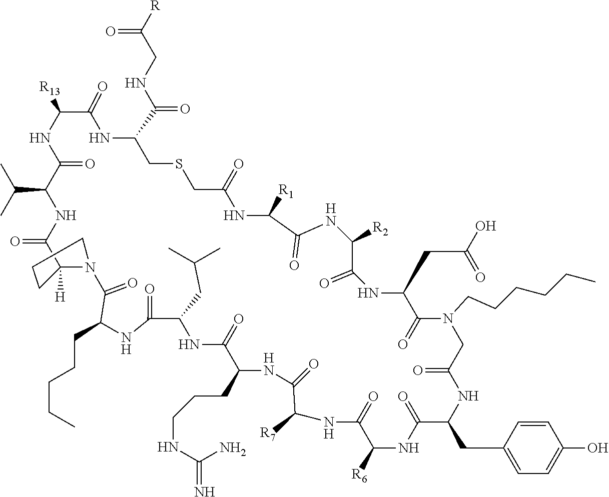

| | | | | | | | | | | | HC |
|---|---|---|---|---|---|---|---|---|---|---|---|
| | | | | | | | | | | | CBA |
| | | | | | | | | QC | Obs. | | mVISTA |
| Ex. | | | | | | | Yield | Method | MS | | IC50 |
| No. | R₁ | R₂ | R₆ | R₇ | R₁₃ | R | (mg) | IDs | Ion | RT | (uM) |
| 1452 | CH₂-1- | CH₂- | nBu | CH₂CONH₂ | CH₂OH | OH | 30.6 | Method | 1905 | 1.7 | 0.0199 |
| | naph- | 4- | | | | | | A | | | |
| | thyl | OH- | | | | | | | | | |
| | | phenyl | | | | | | | | | |
| 1453 | CH₂-2- | CH₂- | nBu | H | CH₂COO | OH | | Method | | | 0.0395 |
| | naph- | 2-CH- | | | H | | | A | | | |
| | thyl | phenyl | | | | | | | | | |
| 1454 | CH₂-2- | CH₂- | nBu | CH₂CONH₂ | CH₂COO | OH | 22.8 | Method | 1970 | 1.77 | 0.0036 |
| | naph- | (N- | | | H | | | B | | | |
| | thyl | Me)- | | | | | | | | | |
| | | indol- | | | | | | | | | |
| | | 3-yl | | | | | | | | | |
| 1455 | CH₂- | CH₂- | nBu | CH₂CONH₂ | CH₂COO | OH | 24.1 | Method | 1920 | 1.56 | 0.0035 |
| | phenyl | (N- | | | H | | | A | | | |
| | | Me)- | | | | | | | | | |
| | | indol- | | | | | | | | | |
| | | 3-yl | | | | | | | | | |
| 1456 | CH₂- | CH₂- | nBu | CH₂CONH₂ | CH₂COO | OH | 28.9 | Method | 1959.9 | 1.53 | 0.0170 |
| | 3,4,5- | indol- | | | H | | | A | | | |
| | triF- | 3-yl | | | | | | | | | |
| | phenyl) | | | | | | | | | | |
| 1457 | CH₂- | CH₂- | nBu | CH₂CONH₂ | CH₂COO | OH | 18.1 | Method | 1937 | 1.56 | 0.0116 |
| | 3,4,5- | 4- | | | H | | | B | | | |
| | triF- | OH- | | | | | | | | | |
| | pheny) | phenyl | | | | | | | | | |
| 1458 | CH₂- | CH₂- | nBu | H | CH₂COO | OH | 28.0 | Method | 1886.07 | 1.4 | 0.0338 |
| | 3,4- | 4- | | | H | | | A | | | |
| | diOMe- | OH- | | | | | | | | | |
| | phenyl | phenyl | | | | | | | | | |
| 1459 | CH₂-4- | CH₂- | nBu | CH₂CONH₂ | CH₂COO | OH | 23.8 | Method | 1974.1 | 1.68 | 0.0234 |
| | CF₃- | indol- | | | H | | | B | | | |
| | phenyl | 3-yl | | | | | | | | | |
| 1460 | CH₂-4- | CH₂- | iBu | CH₂CONH₂ | iPr | OH | 9.6 | Method | 1959.1 | 1.89 | 0.0087 |
| | CF₃- | indol- | | | | | | B | | | |
| | phenyl | 3-yl | | | | | | | | | |
| 1461 | CH₂-4- | CH₂- | nBu | CH₂CONH₂ | CH₂COO | OH | 9.8 | Method | 1951.2 | 1.5 | 0.0050 |
| | CF₃- | 4- | | | H | | | A | | | |
| | phenyl | OH- | | | | | | | | | |
| | | phenyl | | | | | | | | | |
| 1462 | CH₂-4- | CH₂- | nBu | CH₂CONH₂ | CH₂OH | OH | 31.9 | Method | 1873 | 1.62 | 0.0222 |
| | F- | 4- | | | | | | A | | | |
| | phenyl | OH- | | | | | | | | | |
| | | phenyl | | | | | | | | | |
| 1463 | CH₂-4- | CH₂- | iPr | H | CH₂COO | OH | 9.1 | Method | 970 | 1.48 | 0.0230 |
| | F- | 4- | | | H | | | A | | | |
| | phenyl | OH- | | | | | | | | | |
| | | phenyl | | | | | | | | | |
| 1464 | CH₂-4- | CH₂- | nBu | H | CH₂COO | OH | 15.2 | Method | 1841.2 | 1.48 | 0.0314 |
| | NH₂- | 4- | | | H | | | A | | | |
| | phenyl | OH- | | | | | | | | | |
| | | phenyl | | | | | | | | | |
| 1465 | CH₂-3- | CH₂- | iBu | CH₂CONH₂ | iPr | OH | 18.3 | Method | 963.8 | 1.86 | 0.0032 |
| | Br- | indol- | | | | | | B | | | |
| | phenyl | 3-yl | | | | | | | | | |
| 1466 | CH₂-3- | CH₂- | nBu | d | CH₂COO | OH | 17.8 | Method | 972.3 | 1.66 | 0.0033 |
| | Br- | indol- | | CH₂CONH₂ | H | | | B | | | |
| | phenyl | 3-yl | | | | | | | | | |
| 1467 | CH₂-3- | CH₂- | iBu | d | iPr | OH | 13.1 | Method | 978.9 | 1.63 | 0.0169 |
| | Br- | indol- | | CH₂CONH₂ | | | | A | | | |
| | phenyl | 3-yl | | | | | | | | | |
| 1468 | CH₂-3- | CH₂- | iBu | dCH₂CONH₂ | iPr | OH | 16.2 | Method | 952.1 | 1.77 | 0.0046 |
| | Br- | 4- | | | | | | B | | | |
| | phenyl | OH- | | | | | | | | | |
| | | phenyl | | | | | | | | | |
| 1469 | CH₂-3- | CH₂- | nBu | dCH₂CONH₂ | CH₂COO | OH | 22.3 | Method | 1920 | 1.4 | 0.0184 |
| | Br- | 4- | | | H | | | A | | | |
| | phenyl | OH- | | | | | | | | | |
| | | phenyl | | | | | | | | | |
| 1470 | CH₂-3- | CH₂- | iBu | dCH₂CONH₂ | iPr | OH | 9 | Method | 967.1 | 1.56 | 0.0243 |
| | Br- | 4- | | | | | | A | | | |
| | phenyl | OH- | | | | | | | | | |
| | | phenyl | | | | | | | | | |
| 1471 | CH₂- | CH₂- | nBu | dCH₂CONH₂ | CH₂OH | OH | 19.7 | Method | 1894.2 | 1.73 | 0.0329 |
| | indol- | 4- | | | | | | A | | | |
| | 3-yl | OH- | | | | | | | | | |
| | | phenyl | | | | | | | | | |
| 1472 | CH₂-4- | CH₂- | nBu | H | CH₂COO | OH | 21.3 | Method | 1826.2 | 1.73 | 0.0155 |
| | OH- | phenyl | | | H | | | B | | | |
| | phenyl | | | | | | | | | | |
| 1473 | CH₂-4- | CH₂- | nBu | CH₂CONH₂ | CH₂OH | OH | 15.3 | Method | 1871 | 1.62 | 0.0032 |
| | OH- | 4- | | | | | | B | | | |
| | phenyl | OH- | | | | | | | | | |
| | | phenyl | | | | | | | | | |
| 1474 | CH₂-4- | CH₂- | nBu | CH₂CONH₂ | CH₂OH | NH2 | 10.7 | Method | 936.2 | 1.48 | 0.0404 |
| | OH- | 4- | | | | | | A | | | |
| | phenyl | OH- | | | | | | | | | |
| | | phenyl | | | | | | | | | |
| 1475 | CH₂-4- | CH₂- | iPr | H | CH₂COO | NH2 | 5.8 | Method | 934.3 | 1.61 | 0.0107 |
| | Allyl- | 4- | | | H | | | B | | | |
| | phenyl | OH- | | | | | | | | | |
| | | phenyl | | | | | | | | | |
| 1476 | CH₂-4- | CH₂- | nBu | CH₂CONH₂ | CH₂COO | OH | 16.6 | Method | 975.8 | 1.69 | 0.0365 |
| | OEt- | indol- | | 2 | H | | | B | | | |
| | Phenyl | 3-yl | | | | | | | | | |
| 1477 | CH₂-4- | CH₂- | iPr | H | CH₂COO | NH2 | 17 | Method | 921.18 | 1.53 | 0.0492 |
| | OMe- | 4- | | | H | | | B | | | |
| | phenyl | OH- | | | | | | | | | |
| | | phenyl | | | | | | | | | |
| 1478 | CH₂-4- | CH₂- | iPr | H | CH₂COO | OH | 16.2 | Method | 922 | 1.42 | 0.0120 |
| | OMe- | 4- | | | H | | | A | | | |
| | phenyl | OH- | | | | | | | | | |
| | | phenyl | | | | | | | | | |

### Example 2000

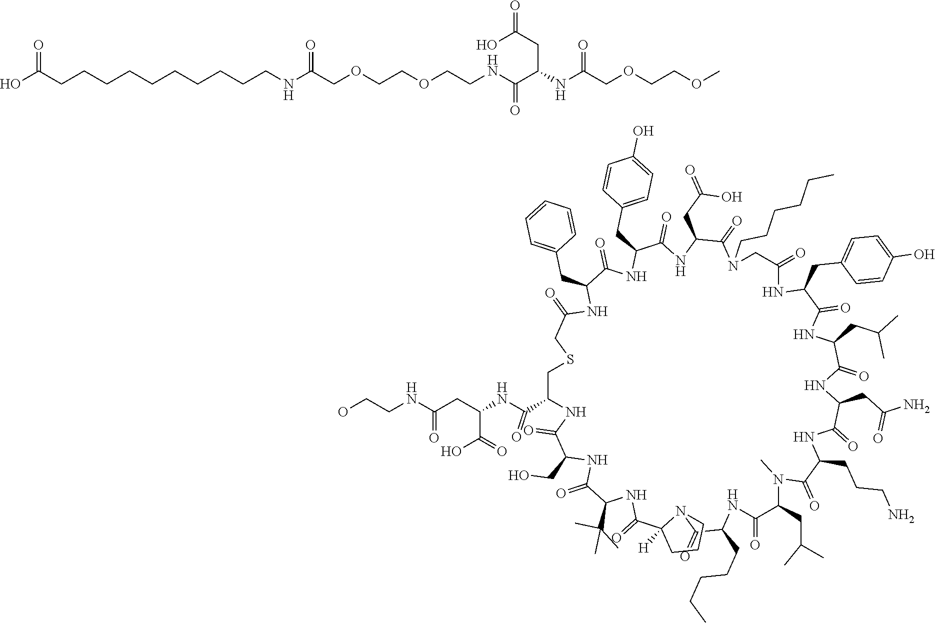 [SMILES: COCCOCC(=O)N[C@@H](CC(=O)O)C(=O)NCCOCCOCC(=O)NCCCCCCCCCCC(=O)O ; CCCCCCN1CC(=O)N[C@@H](Cc2ccc(O)cc2)C(=O)N[C@@H](CC(C)C)C(=O)N[C@@H](CC(N)=O)C(=O)N[C@@H](CCCN)C(=O)N(C)[C@@H](CC(C)C)C(=O)N[C@@H](CCCCC)C(=O)N2CCC[C@H]2C(=O)N[C@@H](C(C)(C)C)C(=O)N[C@@H](CO)C(=O)N[C@H](C(=O)N[C@@H](CC(=O)NCCO)C(=O)O)CSCC(=O)N[C@@H](Cc2ccccc2)C(=O)N[C@@H](Cc2ccc(O)cc2)C(=O)N[C@@H](CC(=O)O)C1=O]

Example 2000 was prepared, using chlorotrityl resin preloaded with FAA11 (0.238 mmol/g loading) on a 50 μmol scale, following the general synthetic sequence described for the preparation of Example 1000. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 23 minutes, then a 4-minute hold at 100% B; Flow Rate: 20 mL/min; Column Temperature: 25 C. Fraction collection was triggered by UV signals. Fractions containing the desired product were combined and dried via centrifugal evaporation. The yield of the product was 22.7 mg, and its estimated purity by LCMS analysis was 96%.

Retention time=1.47 min;ESI-MS(+)m/z[M+2H]²⁺:1244.3. Analysis condition A

Retention time=1.78 min;ESI-MS(+)m/z[M+2H]²⁺:1245.0. Analysis condition B

The following examples were prepared according to the procedures similar to the one described for Example 2000.

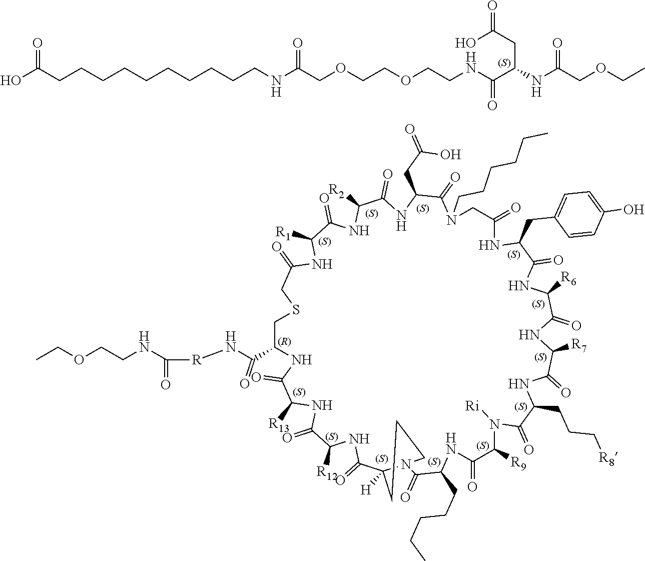

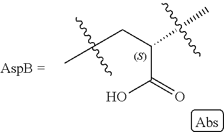

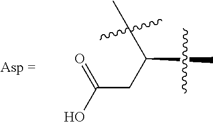

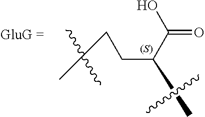

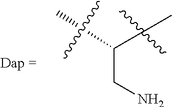

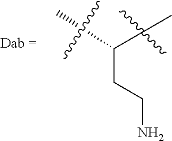

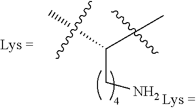

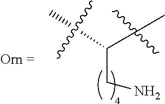

| | | | | | | | | | | | | | | HC |
|---|---|---|---|---|---|---|---|---|---|---|---|---|---|---|
| | | | | | | | | | | | | | | CBA |
| | | | | | | | | | | | QC | | | miPr |
| Ex. | | | | | | | | | | | Meth- | Obs. | | ISTA |
| Num- | | | | | | Rᵢ, | | | | Yield | od | MS | | IC50 |
| bero. | R₁ | R₂ | R₆ | R₇ | R_{8′} | R₉ | R₁₂ | R₁₃ | R₁₅ | (mg) | IDs | Ion | RT | (uM) |
| 2000 | CH₂- | CH₂- | iBu | CH₂C | NH₂ | Me, | tBu | CH₂OH | AspB | 22.7 | A | 1244.3 | 1.47 | 0.0161 |
| | phenyl | 4- | | ONH₂ | | iBu | | | | | | | | |
| | | OH- | | | | | | | | | | | | |
| | | phenyl | | | | | | | | | | | | |
| 2001 | CH₂- | CH₂- | iBu | CH₂C | guani- | H, | iPr | CH₂OH | CH₂ | 32.2 | A | 1221.9 | 1.57 | 0.0198 |
| | phenyl | 4- | | ONH₂ | dinyl | iBu | | | | | | | | |
| | | OH- | | | | | | | | | | | | |
| | | phenyl | | | | | | | | | | | | |
| 2002 | CH₂- | CH₂- | iBu | CH₂C | guani- | H, | iPr | CH₂OH | GluG | 30 | A | 1258.2 | 1.47 | 0.0196 |
| | phenyl | 4- | | ONH₂ | dinyl | iBu | | | | | | | | |
| | | OH- | | | | | | | | | | | | |
| | | phenyl | | | | | | | | | | | | |
| 2003 | CH₂- | CH₂- | iBu | CH₂C | guani- | H, | iPr | CH₂OH | AspB | 26.3 | A | 1251.1 | 1.48 | 0.0378 |
| | phenyl | 4- | | ONH₂ | dinyl | iBu | | | | | | | | |
| | | OH- | | | | | | | | | | | | |
| | | phenyl | | | | | | | | | | | | |
| 2004 | CH₂- | CH₂- | iBu | CH₂C | guani- | H, | iPr | CH₂OH | Asp | 24.6 | B | 1251.1 | 1.61 | 0.0493 |
| | phenyl | 4- | | ONH₂ | dinyl | iBu | | | | | | | | |
| | | OH- | | | | | | | | | | | | |
| | | phenyl | | | | | | | | | | | | |
| 2005 | CH₂- | CH₂- | nBu | CH₂C | guani- | H, | iPr | CH₂C | CH₂ | 14.9 | A | 1236.9 | 1.39 | 0.0105 |
| | phenyl | 4- | | OOH | dinyl | iBu | | ONH2 | | | | | | |
| | | OH- | | | | | | | | | | | | |
| | | phenyl | | | | | | | | | | | | |
| 2006 | CH₂- | CH₂- | nBu | CH₂C | guani- | Me | iPr | CH₂C | CH₂ | 17.6 | B | 853.6 | 1.77 | 0.0285 |
| | 2- | 4- | | ONH₂ | dinyl | nBu | | OOH | | | | | | |
| | naph- | OH- | | | | | | | | | | | | |
| | thyl | phenyl | | | | | | | | | | | | |
| 2007 | CH₂- | CH₂- | nBu | CH₂C | guani- | H, | iPr | CH₂OH | CH₂ | 20.4 | A | 1247.5 | 1.59 | 0.0196 |
| | 2- | 4- | | ONH₂ | dinyl | iBu | | | | | | | | |
| | naph- | OH- | | | | | | | | | | | | |
| | thyl | phenyl | | | | | | | | | | | | |
| 2008 | CH₂- | CH₂- | nBu | CH₂C | guani- | H, | iPr | CH₂C | CH₂ | 17.4 | B | 837.2 | 1.77 | 0.0161 |
| | 3- | indol- | | ONH₂ | dinyl | iBu | | OOH | | | | | | |
| | Me- | 3-yl | | | | | | | | | | | | |
| | phenyl | | | | | | | | | | | | | |
| 2009 | CH₂- | CH₂- | iBu | CH₂C | guani- | H, | iPr | iPr | CH₂ | 18.6 | B | 818.3 | 2.1 | 0.0389 |
| | phenyl | 3- | | ONH₂ | dinyl | iBu | | | | | | | | |
| | | Me- | | | | | | | | | | | | |
| | | phenyl | | | | | | | | | | | | |
| 2010 | CH₂- | CH₂- | Nle | CH₂C | guani- | Me, | iPr | CH₂C | CH₂ | 20.1 | B | 837 | 1.85 | 0.0159 |
| | phenyl | indol- | | ONH₂ | dinyl | nBu | | OOH | | | | | | |
| | | 3-yl | | | | | | | | | | | | |
| 2011 | CH₂- | CH₂- | iPr | H | guani- | H, | iPr | CH₂C | CH₂ | 26.7 | B | 828.1 | 1.67 | 0.0241 |
| | 2- | 4- | | | dinyl | iBu | | OOH | | | | | | |
| | naph- | OH- | | | | | | | | | | | | |
| | thyl | phenyl | | | | | | | | | | | | |
| 2012 | CH₂- | CH₂- | iBu | CH₂C | guani- | H, | iPr | iPr | CH₂ | 4.5 | A | 1229.2 | 1.72 | 0.0513 |
| | phenyl | 4- | | ONH₂ | dinyl | iBu | | | | | | | | |
| | | OH- | | | | | | | | | | | | |
| | | phenyl | | | | | | | | | | | | |
| 2013 | CH₂- | CH₂- | n- | H | guani- | H, | iPr | CH₂OH | CH₂ | 20.8 | B | 1201.9 | 1.79 | 0.0255 |
| | phenyl | 4- | Pentyl | | dinyl | iBu | | | | | | | | |
| | | OH- | | | | | | | | | | | | |
| | | phenyl | | | | | | | | | | | | |
| 2014 | CH₂- | CH₂- | nBu | H | guani- | H, | iPr | CH₂C | CH₂ | 20.2 | B | 810.1 | 1.84 | 0.0164 |
| | 3- | 4- | | | dinyl | iBu | | OOH | | | | | | |
| | Me- | OH- | | | | | | | | | | | | |
| | phenyl | phenyl | | | | | | | | | | | | |
| 2015 | CH₂- | CH₂- | nBu | CH₂C | NHC | Me, | iPr | CH₂C | CH₂ | 23.8 | B | 1256 | 1.85 | 0.0205 |
| | phenyl | 4- | | ONH₂ | ONH₂ | iBu | | OOH | | | | | | |
| | | OH- | | | | | | | | | | | | |
| | | phenyl | | | | | | | | | | | | |
| 2016 | CH₂- | CH₂- | nBu | CH₂C | NHC | H, | iPr | CH₂OH | CH₂ | 13.8 | A | 1248.1 | 1.65 | 0.0190 |
| | 2- | 4- | | ONH₂ | ONH₂ | iBu | | | | | | | | |
| | naph- | OH- | | | | | | | | | | | | |
| | thyl | phenyl | | | | | | | | | | | | |
| 2017 | CH₂- | CH₂- | nBu | CH₂C | NHC | H, | iPr | CH₂C | CH₂ | 29.6 | A | 1255.3 | 1.64 | 0.0111 |
| | 3- | 4- | | ONH₂ | ONH₂ | iBu | | OOH | | | | | | |
| | Me- | OH- | | | | | | | | | | | | |
| | phenyl | phenyl | | | | | | | | | | | | |
| 2018 | CH₂- | CH₂- | nBu | CH₂C | NHC | H, | iPr | CH₂C | CH₂ | 4.7 | B | 1237 | 1.47 | 0.0204 |
| | phenyl | 4- | | OOH | ONH₂ | iBu | | ONH₂ | | | | | | |
| | | OH- | | | | | | | | | | | | |
| | | phenyl | | | | | | | | | | | | |
| 2019 | CH₂- | CH₂- | nBu | H | NHC | H, | iPr | CH₂C | CH₂ | 35.2 | A | 1216.1 | 1.48 | 0.0144 |
| | 3- | 4- | | | ONH₂ | iBu | | OOH | | | | | | |
| | Me- | OH- | | | | | | | | | | | | |
| | phenyl | phenyl | | | | | | | | | | | | |
| 2020 | CH₂- | CH₂- | nBu | CH₂C | guani- | Me, | tBu | CH₂OH | AspB | 42.5 | B | 843.26 | 1.96 | 0.0227 |
| | phenyl | 4- | | ONH₂ | dinyl | nBu | | | | | | | | |
| | | OH- | | | | | | | | | | | | |
| | | phenyl | | | | | | | | | | | | |
| 2021 | CH₂- | CH₂- | nBu | CH₂C | guani- | Me, | tBu | CH₂OH | AspB | 40.1 | B | 1266.02 | 1.82 | 0.0195 |
| | phenyl | 4- | | ONH₂ | dinyl | nBu | | | | | | | | |
| | | OH- | | | | | | | | | | | | |
| | | phenyl | | | | | | | | | | | | |
| 2022 | CH₂- | CH₂- | nBu | CH₂C | guani- | Me, | tBu | CH₂OH | AspB | 21 | A | 1276.8 | 1.55 | 0.0227 |
| | phenyl | indol- | | ONH₂ | dinyl | nBu | | | | | | | | |
| | | 3- | | | | | | | | | | | | |
| | | yl | | | | | | | | | | | | |
| 2023 | CH2- | CH₂- | nBu | CH₂C | guani- | Me, | tBu | CH₂OH | AspB | 33.7 | A | 1290.99, | 1.56, | 0.0412 |
| | 2- | 4- | | ONH₂ | dinyl | nBu | | | | | | 1291.02 | 1.64 | |
| | naph- | OH- | | | | | | | | | | | | |
| | thyl | phenyl | | | | | | | | | | | | |
| 2024 | CH₂- | CH₂- | nBu | CH₂C | guani- | Me, | tBu | CH₂OH | AspB | 3.9 | B | 1284.3 | 1.82 | 0.0403 |
| | 3- | indol- | | ONH₂ | dinyl | nBu | | | | | | | | |
| | Me- | 3- | | | | | | | | | | | | |
| | phenyl | yl | | | | | | | | | | | | |
| 2025 | CH₂- | CH₂- | iBu | CH₂C | guani- | Me, | tBu | CH₂OH | AspB | 9.8 | B | 1264 | 1.82 | 0.0345 |
| | phenyl | indol- | | ONH₂ | dinyl | CH₂ | | | | | | | | |
| | | 3- | | | | OOH | | | | | | | | |
| | | yl | | | | | | | | | | | | |
| 2026 | CH₂- | CH₂- | iBu | CH₂C | guani- | Me, | tBu | CH₂C | AspB | 8.6 | B | 1280.1 | 1.8 | 0.0175 |
| | phenyl | 4- | | ONH₂ | dinyl | iBu | | OOH | | | | | | |
| | | OH- | | | | | | | | | | | | |
| | | phenyl | | | | | | | | | | | | |
| 2027 | CH₂- | CH₂- | iBu | CH₂C | guani- | Me, | tBu | CH₂C | AspB | 8.8 | A | 1291 | 1.47 | 0.0275 |
| | phenyl | indol- | | ONH₂ | dinyl | nBu | | OOH | | | | | | |
| | | 3- | | | | | | | | | | | | |
| | | yl | | | | | | | | | | | | |
| 2028 | CH₂- | CH₂- | iBu | CH₂C | guani- | Me, | tBu | CH₂OH | AspB | 21.7 | B | 1276.97, | 1.91, | 0.0166 |
| | phenyl | indol- | | ONH₂ | dinyl | nBu | | | | | | 1276.97 | 2.01 | |
| | | 3- | | | | | | | | | | | | |
| | | yl | | | | | | | | | | | | |
| 2029 | CH₂- | CH₂- | iBu | CH₂C | guani- | Me, | tBu | CH₂C | AspB | 10.3 | A | 1280 | 1.49 | 0.0175 |
| | phenyl | 4- | | ONH₂ | dinyl | nBu | | OOH | | | | | | |
| | | OH- | | | | | | | | | | | | |
| | | phenyl | | | | | | | | | | | | |
| 2030 | CH₂- | CH₂- | iBu | CH₂C | guani- | Me, | tBu | CH₂OH | AspB | 6.5 | A | 1266.1 | 1.49 | 0.0228 |
| | phenyl | 4- | | ONH₂ | dinyl | nBu | | | | | | | | |
| | | OH- | | | | | | | | | | | | |
| | | phenyl | | | | | | | | | | | | |
| 2031 | CH₂- | CH₂- | iBu | CH₂C | guani- | Me, | tBu | CH₂OH | AspB | 20.2 | B | 844.1 | 1.78 | 0.0145 |
| | phenyl | 4- | | ONH₂ | dinyl | iBu | | | | | | | | |
| | | OH- | | | | | | | | | | | | |
| | | phenyl | | | | | | | | | | | | |
| 2032 | CH₂- | CH₂- | iBu | CH₂C | guani- | Me, | tBu | CH₂C | AspB | 12.5 | B | 1291 | 1.88 | 0.0281 |
| | phenyl | indol- | | ONH₂ | dinyl | iBu | | OOH | | | | | | |
| | | 3- | | | | | | | | | | | | |
| | | yl | | | | | | | | | | | | |
| 2033 | CH₂- | CH₂- | iBu | CH₂C | guani- | Me, | tBu | CH₂OH | AspB | 13.7 | A | 835.2 | 1.37 | 0.0184 |
| | phenyl | 4- | | ONH₂ | dinyl | CH₂ | | | | | | | | |
| | | OH- | | | | OOH | | | | | | | | |
| | | phenyl | | | | | | | | | | | | |
| 2034 | CH₂- | CH₂- | iBu | CH₂C | guani- | Me, | tBu | CH₂C | AspB | 6.7 | B | 1278.1 | 1.8 | 0.0435 |
| | phenyl | indol- | | ONH₂ | dinyl | CH₂ | | OOH | | | | | | |
| | | 3- | | | | OOH | | | | | | | | |
| | | yl | | | | | | | | | | | | |
| 2035 | CH₂- | CH₂- | iBu | CH₂C | guani- | Me, | tBu | CH₂C | AspB | 3.2 | A | 1267.2 | 1.36 | 0.0371 |
| | phenyl | 4- | | ONH₂ | dinyl | CH₂ | | OOH | | | | | | |
| | | OH- | | | | OOH | | | | | | | | |
| | | phenyl | | | | | | | | | | | | |
| 2036 | CH₂- | CH₂- | iBu | CH₂C | guani- | Me, | tBu | CH₂OH | AspB | 4.4 | A | 1259.8 | 1.4 | 0.0490 |
| | phenyl | 4- | | ONH₂ | dinyl | CH | | | | | | | | |
| | | OH- | | | | MeO | | | | | | | | |
| | | phenyl | | | | H | | | | | | | | |
| 2037 | CH₂- | CH₂- | iBu | CH₂C | guani- | Me, | tBu | CH₂C | AspB | 2.2 | A | 1272 | 1.35 | 0.0344 |
| | phenyl | 4- | | ONH₂ | dinyl | iPr | | OOH | | | | | | |
| | | OH- | | | | | | | | | | | | |
| | | phenyl | | | | | | | | | | | | |
| 2038 | CH₂- | CH₂- | iBu | CH₂C | guani- | Me, | tBu | CH₂OH | AspB | 10.3 | A | 1273.3 | 1.28 | 0.0341 |
| | phenyl | 4- | | ONH₂ | dinyl | CH | | | | | | | | |
| | | OH- | | | | MeO | | | | | | | | |
| | | phenyl | | | | H | | | | | | | | |
| 2039 | CH₂- | CH₂- | iBu | CH₂C | guani- | Me, | tBu | CH₂OH | AspB | 5.8 | A | 1270.4 | 1.54 | 0.0238 |
| | phenyl | indol- | | ONH₂ | dinyl | iPr | | | | | | | | |
| | | 3- | | | | | | | | | | | | |
| | | yl | | | | | | | | | | | | |
| 2040 | CH₂- | CH₂- | iBu | CH₂C | guani- | Me, | tBu | CH₂C | AspB | 2 | A | 1283 | 1.42 | 0.0407 |
| | phenyl | indol- | | ONH₂ | dinyl | iPr | | OOH | | | | | | |
| | | 3- | | | | | | | | | | | | |
| | | yl | | | | | | | | | | | | |
| 2041 | CH₂- | CH₂- | iBu | CH₂C | guani- | Me, | tBu | CH₂OH | AspB | 2.8 | B | 1258.2 | 1.73 | 0.0297 |
| | phenyl | 4- | | ONH₂ | dinyl | iPr | | | | | | | | |
| | | OH- | | | | | | | | | | | | |
| | | phenyl | | | | | | | | | | | | |
| 2042 | CH₂- | CH₂- | iBu | CH₂C | guani- | Me, | tBu | CH₂C | AspB | 2.3 | A | 1285.2 | 1.33 | 0.0308 |
| | phenyl | indol- | | ONH₂ | dinyl | CH | | OOH | | | | | | |
| | | 3- | | | | MeO | | | | | | | | |
| | | yl | | | | H | | | | | | | | |
| 2043 | CH₂- | CH₂- | iBu | CH₂C | NH₂ | Me, | tBu | CH₂OH | AspB | 9.8 | A | 1243.1 | 1.55 | 0.0329 |
| | phenyl | indol- | | ONH₂ | | CH₂ | | | | | | | | |
| | | 3- | | | | OOH | | | | | | | | |
| | | yl | | | | | | | | | | | | |
| 2044 | CH₂- | CH₂- | iBu | CH₂C | NH₂ | Me, | tBu | CH₂C | AspB | 28.9 | B | 1245.9 | 1.68 | 0.0412 |
| | phenyl | 4- | | ONH₂ | | CH₂ | | OOH | | | | | | |
| | | OH- | | | | OOH | | | | | | | | |
| | | phenyl | | | | | | | | | | | | |
| 2045 | CH₂- | CH₂- | iBu | CH₂C | NH₂ | Me, | tBu | CH₂OH | AspB | 19.6 | A | 1245 | 1.43 | 0.0201 |
| | phenyl | 4- | | ONH₂ | | nBu | | | | | | | | |
| | | OH- | | | | | | | | | | | | |
| | | phenyl | | | | | | | | | | | | |
| 2046 | CH₂- | CH₂- | (CH₂)₄ | CH₂C | guani- | Me, | tBu | CH₂OH | AspB | 11.6 | A | 1273.3 | 1.67 | 0.0345 |
| | phenyl | 4- | NH₂ | ONH₂ | dinyl | nBu | | | | | | | | |
| | | OH- | | | | | | | | | | | | |
| | | phenyl | | | | | | | | | | | | |
| 2047 | CH₂- | CH₂- | iBu | CH₂C | NH₂ | Me, | tBu | CH₂OH | AspB | 23.6 | A | 1267.2 | 1.49 | 0.0488 |
| | phenyl | 4- | | ONH₂ | | nBu | | | | | | | | |
| | (4- | OH- | | | | | | | | | | | | |
| | COOH) | phenyl | | | | | | | | | | | | |
| 2048 | CH₂- | CH₂- | iBu | CH₂C | guani- | Me, | tBu | CH₂C | AspB | 32.3 | A | 1288.2 | 1.32 | 0.0350 |
| | phenyl | 4- | | ONH₂ | dinyl | CH₂ | | OOH | | | | | | |
| | (4- | OH- | | | | OOH | | | | | | | | |
| | COOH) | phenyl | | | | | | | | | | | | |
| 2049 | CH₂- | CH₂- | iBu | CH₂C | NH₂ | Me, | tBu | CH₂OH | AspB | 29 | A | 1266.2 | 1.44 | 0.0288 |
| | phenyl | 4- | | ONH₂ | | iBu | | | | | | | | |
| | (4- | OH- | | | | | | | | | | | | |
| | COOH) | phenyl | | | | | | | | | | | | |
| 2050 | CH₂- | CH₂- | iBu | CH₂C | guani- | Me, | tBu | CH₂OH | AspB | 16.9 | B | 858.6 | 1.68 | 0.0241 |
| | phenyl | 4- | | ONH₂ | dinyl | nBu | | | | | | | | |
| | (4- | OH- | | | | | | | | | | | | |
| | COOH) | phenyl | | | | | | | | | | | | |
| 2051 | CH₂- | CH₂- | iBu | CH₂C | NH₂ | Me, | tBu | CH₂OH | AspB | 20.7 | A | 1265.1 | 1.33 | 0.0406 |
| | phenyl | indol- | | ONH₂ | | CH₂ | | | | | | | | |
| | (4- | 3- | | | | OOH | | | | | | | | |
| | COOH) | yl | | | | | | | | | | | | |
| 2052 | CH₂- | CH₂- | iBu | CH₂C | guani- | Me, | tBu | CH₂OH | AspB | 18.1 | B | 859 | 1.67 | 0.0317 |
| | phenyl | 4- | | ONH₂ | dinyl | iBu | | | | | | | | |
| | (4- | OH- | | | | | | | | | | | | |
| | COOH) | phenyl | | | | | | | | | | | | |
| 2053 | CH₂- | CH₂- | CH₂- | CH₂C | guani- | Me, | tBu | CH₂OH | AspB | 24.7 | A | 1286.3 | 1.67 | 0.0394 |
| | phenyl | 4- | cyclo- | ONH₂ | dinyl | nBu | | | | | | | | |
| | | OH- | hexyl | | | | | | | | | | | |
| | | phenyl | | | | | | | | | | | | |
| 2054 | CH₂- | CH₂- | (CH₂)₃ | CH₂C | guani- | Me, | tBu | CH₂OH | AspB | 13.1 | A | 1281.1 | 1.35 | 0.0232 |
| | phenyl | 4- | COOH | ONH₂ | dinyl | nBu | | | | | | | | |
| | | OH- | | | | | | | | | | | | |
| | | phenyl | | | | | | | | | | | | |
| 2055 | CH₂- | CH₂- | CH₂- | CH₂C | NH₂ | Me, | tBu | CH₂OH | AspB | 23.6 | A | 1265 | 1.59 | 0.0327 |
| | phenyl | 4- | cyclo- | ONH₂ | | nBu | | | | | | | | |
| | | OH- | hexyl | | | | | | | | | | | |
| | | phenyl | | | | | | | | | | | | |
| 2056 | CH₂- | CH₂- | CH₂)₃ | CH₂C | NH₂ | Me, | tBu | CH₂OH | AspB | 16.5 | B | 1260 | 1.66 | 0.0261 |
| | phenyl | 4- | COOH | ONH₂ | | nBu | | | | | | | | |
| | | OH- | | | | | | | | | | | | |
| | | phenyl | | | | | | | | | | | | |
| 2057 | CH₂- | CH₂- | iBu | CH₂C | NH₂ | Me, | tBu | CH₂OH | AspB | 30.6 | B | 1238 | 1.71 | 0.0099 |
| | phenyl | 4- | | ONH₂ | | nBu | | | | | | | | |
| | | OH- | | | | | | | | | | | | |
| | | phenyl | | | | | | | | | | | | |
| 2058 | CH₂- | CH₂- | Me | CH₂C | guani- | Me, | tBu | CH₂OH | AspB | 11.1 | A | 830 | 1.36 | 0.0276 |
| | phenyl | 4- | | ONH₂ | dinyl | nBu | | | | | | | | |
| | | OH- | | | | | | | | | | | | |
| | | phenyl | | | | | | | | | | | | |
| 2059 | CH₂- | CH₂- | Et | CH₂C | guani- | Me, | tBu | CH₂OH | AspB | 15 | A | 1252 | 1.43 | 0.0374 |
| | phenyl | 4- | | ONH₂ | dinyl | nBu | | | | | | | | |
| | | OH- | | | | | | | | | | | | |
| | | phenyl | | | | | | | | | | | | |
| 2060 | CH₂- | CH₂- | Et | CH₂C | NH₂ | Me, | tBu | CH₂OH | AspB | 19.7 | A | 1230.2 | 1.46 | 0.0247 |
| | phenyl | 4- | | ONH₂ | | nBu | | | | | | | | |
| | | OH- | | | | | | | | | | | | |
| | | phenyl | | | | | | | | | | | | |
| 2061 | CH₂- | CH₂- | Me | CH₂C | NH₂ | Me, | tBu | CH₂OH | AspB | 15.9 | B | 816 | 1.75 | 0.0215 |
| | phenyl | 4- | | ONH₂ | | nBu | | | | | | | | |
| | | OH- | | | | | | | | | | | | |
| | | phenyl | | | | | | | | | | | | |
| 2062 | CH₂- | CH₂- | CH₂- | CH₂C | NH₂ | Me, | tBu | CH₂OH | AspB | 5.4 | A | 1243.9 | 1.41 | 0.0492 |
| | phenyl | 4- | cPr | ONH₂ | | nBu | | | | | | | | |
| | | OH- | | | | | | | | | | | | |
| | | phenyl | | | | | | | | | | | | |
| 2063 | CH₂- | CH₂- | CH₂- | CH₂C | guani- | Me, | tBu | CH₂OH | AspB | 3.1 | A | 1265 | 1.41 | 0.0399 |
| | phenyl | 4- | cPr | ONH₂ | dinyl | nBu | | | | | | | | |
| | | OH- | | | | | | | | | | | | |
| | | phenyl | | | | | | | | | | | | |
| 2065 | CH₂- | CH₂- | iBu | CH₂C | NH₂ | Me, | tBu | CH₂C | AspB | 26.9 | A | 1245.9 | 1.19 | 0.0295 |
| | phenyl | 4- | | OOH | | CH₂ | | ONH₂ | | | | | | |
| | | OH- | | | | OOH | | | | | | | | |
| | | phenyl | | | | | | | | | | | | |
| 2066 | CH₂- | CH₂- | iBu | CH₂C | guani- | Me, | tBu | CH₂C | AspB | 20.5 | A | 1278.1 | 1.45 | 0.0353 |
| | phenyl | indol- | | OOH | dinyl | CH₂ | | ONH₂ | | | | | | |
| | | 3- | | | | OOH | | | | | | | | |
| | | yl | | | | | | | | | | | | |
| 2067 | CH₂- | CH₂- | iBu | CH₂C | NH₂ | Me, | tBu | CH₂C | AspB | 36.4 | A | 1257.3 | 1.33 | 0.0403 |
| | phenyl | indol- | | OOH | | CH₂ | | ONH₂ | | | | | | |
| | | 3- | | | | OOH | | | | | | | | |
| | | yl | | | | | | | | | | | | |
| 2068 | CH₂- | CH₂- | iBu | CH₂C | guani- | Me, | tBu | CH₂OH | AspB | 13.1 | A | 1252.4 | 1.29 | 0.0299 |
| | phenyl | 4- | | ONH₂ | dinyl | CH₂ | | | | | | | | |
| | | OH- | | | | OOH | | | | | | | | |
| | | phenyl | | | | | | | | | | | | |
| 2069 | CH₂- | CH₂- | iBu | CH₂C | guani- | Me, | tBu | CH₂C | AspB | 7.6 | B | 844.1 | 1.78 | 0.0450 |
| | phenyl | 3- | | ONH₂ | dinyl | CH₂ | | OOH | | | | | | |
| | | Me- | | | | OOH | | | | | | | | |
| | | phenyl | | | | | | | | | | | | |
| 2070 | CH₂- | CH₂- | iBu | CH₂C | guani- | Me, | tBu | CH₂C | AspB | 5.8 | B | 1292.4 | 1.71 | 0.0416 |
| | 2- | 4- | | ONH₂ | dinyl | CH₂ | | OOH | | | | | | |
| | naph- | OH- | | | | OOH | | | | | | | | |
| | thyl | phenyl | | | | | | | | | | | | |
| 2071 | CH₂- | CH₂- | iBu | CH₂C | guani- | Me, | tBu | CH₂C | AspB | 3.1 | A | 849.1 | 1.38 | 0.0278 |
| | 3- | 4- | | ONH₂ | dinyl | CH₂ | | OOH | | | | | | |
| | Me- | OH- | | | | OOH | | | | | | | | |
| | phenyl | phenyl | | | | | | | | | | | | |
| 2072 | CH₂- | CH₂- | iBu | CH₂C | NH₂ | Me, | tBu | iPr | AspB | 51.4 | A | 1249.2 | 1.6 | 0.0211 |
| | phenyl | 3- | | ONH₂ | | nBu | | | | | | | | |
| | | Me- | | | | | | | | | | | | |
| | | phenyl | | | | | | | | | | | | |
| 2073 | CH₂- | CH₂- | iBu | CH₂C | guani- | Me, | tBu | iPr | AspB | 24.5 | A | 1271 | 1.61 | 0.0334 |
| | phenyl | 3- | | ONH₂ | dinyl | nBu | | | | | | | | |
| | | Me- | | | | | | | | | | | | |
| | | phenyl | | | | | | | | | | | | |
| 2074 | CH₂- | CH₂- | iBu | CH₂C | NH₂ | Me, | tBu | CH₂C | AspB | 30.3 | B | 1258 | 1.94 | 0.0400 |
| | phenyl | 3- | | ONH₂ | | nBu | | OOH | | | | | | |
| | | Me- | | | | | | | | | | | | |
| | | phenyl | | | | | | | | | | | | |
| 2075 | CH₂- | CH₂- | iBu | CH₂C | guani- | Me, | tBu | CH₂C | AspB | 12.4 | A | 852.9 | 1.58 | 0.0241 |
| | phenyl | 3- | | ONH₂ | dinyl | nBu | | OOH | | | | | | |
| | | Me- | | | | | | | | | | | | |
| | | phenyl | | | | | | | | | | | | |
| 2076 | CH₂- | CH₂- | iBu | CH₂C | guani- | Me, | tBu | CH₂OH | AspB | 20.7 | B | 1264.2 | 1.96 | 0.0381 |
| | phenyl | 3- | | ONH₂ | dinyl | nBu | | | | | | | | |
| | | Me- | | | | | | | | | | | | |
| | | phenyl | | | | | | | | | | | | |
| 2077 | CH₂- | CH₂- | iBu | CH₂C | NH₂ | Me, | tBu | CH₂OH | AspB | 26 | B | 1243.2 | 1.96 | 0.0335 |
| | phenyl | 3- | | ONH₂ | | nBu | | | | | | | | |
| | | Me- | | | | | | | | | | | | |
| | | phenyl | | | | | | | | | | | | |
| 2078 | CH₂- | CH₂- | iBu | CH₂C | guani- | Me, | tBu | CH₂OH | AspB | 20.7 | A | 1251.1 | 1.59 | 0.0402 |
| | phenyl | 4- | | ONH₂ | dinyl | nBu | | | | | | | | |
| | | OH- | | | | | | | | | | | | |
| | | phenyl | | | | | | | | | | | | |
| 2079 | CH₂- | CH₂- | iBu | CH₂C | guani- | Me, | tBu | CH₂C | AspB | 26.5 | B | 1286.1 | 1.74 | 0.0154 |
| | phenyl | 4- | | ONH₂ | dinyl | nBu | | OOH | | | | | | |
| | | OH- | | | | | | | | | | | | |
| | | phenyl | | | | | | | | | | | | |
| 2080 | CH₂- | CH₂- | iBu | CH₂C | guani- | Me, | tBu | CH₂OH | Lys | 31.1 | B | 1272 | 1.75 | 0.0285 |
| | phenyl | 4- | | ONH₂ | dinyl | nBu | | | | | | | | |
| | | OH- | | | | | | | | | | | | |
| | | phenyl | | | | | | | | | | | | |
| 2081 | CH₂- | CH₂- | iBu | CH₂C | guani- | Me, | tBu | CH₂C | Lys | 31.7 | B | 1286.2 | 1.72 | 0.0277 |
| | phenyl | 4- | | ONH₂ | dinyl | nBu | | OOH | | | | | | |
| | | OH- | | | | | | | | | | | | |
| | | phenyl | | | | | | | | | | | | |
| 2082 | CH₂- | CH₂- | iBu | CH₂C | guani- | Me, | tBu | CH₂C | Orn | 17.3 | B | 853.2 | 1.56 | 0.0276 |
| | phenyl | 4- | | ONH₂ | dinyl | nBu | | OOH | | | | | | |
| | | OH- | | | | | | | | | | | | |
| | | phenyl | | | | | | | | | | | | |
| 2083 | CH₂- | CH₂- | iBu | CH₂C | guani- | Me, | tBu | CH₂C | Dap | 25.6 | B | 1265.2 | 1.72 | 0.0437 |
| | phenyl | 4- | | ONH₂ | dinyl | nBu | | OOH | | | | | | |
| | | OH- | | | | | | | | | | | | |
| | | phenyl | | | | | | | | | | | | |
| 2084 | CH₂- | CH₂- | iBu | CH₂C | guani- | Me, | tBu | CH₂OH | AspB | 19.2 | A | 1272.2 | 1.53 | 0.0268 |
| | phenyl | 4- | | ONH₂ | dinyl | nBu | | | | | | | | |
| | | OH- | | | | | | | | | | | | |
| | | phenyl | | | | | | | | | | | | |
| 2085 | CH₂- | CH₂- | iBu | CH₂C | guani- | Me, | tBu | CH₂C | Dab | 19.1 | B | 1272.1 | 1.73 | 0.0417 |
| | phenyl | 4- | | ONH₂ | dinyl | nBu | | OOH | | | | | | |
| | | OH- | | | | | | | | | | | | |
| | | phenyl | | | | | | | | | | | | |
| 2086 | CH₂- | CH₂- | iBu | CH₂C | guani- | Me, | tBu | CH₂OH | Dab | 16.2 | A | 1258.1 | 1.55 | 0.0499 |
| | phenyl | 4- | | ONH₂ | dinyl | nBu | | | | | | | | |
| | | OH- | | | | | | | | | | | | |
| | | phenyl | | | | | | | | | | | | |
| 2087 | CH₂- | CH₂- | iBu | CH₂C | guani- | Me, | tBu | CH₂OH | AspB | 20.7 | B | 1287.1 | 1.73 | 0.0288 |
| | phenyl | 4- | | ONH₂ | dinyl | nBu | | | | | | | | |
| | (4- | OH- | | | | | | | | | | | | |
| | CO | phenyl | | | | | | | | | | | | |
| | NH₂) | | | | | | | | | | | | | |
| 2088 | CH₂- | CH₂- | iBu | CH₂C | guani- | Me, | tBu | CH₂C | AspB | 15.4 | A | 1301.2 | 1.28 | 0.0169 |
| | phenyl | 4- | | ONH₂ | dinyl | nBu | | OOH | | | | | | |
| | (4- | OH- | | | | | | | | | | | | |
| | CO | phenyl | | | | | | | | | | | | |
| | NH₂) | | | | | | | | | | | | | |
| 2089 | CH₂- | CH₂- | iBu | CH₂C | NH₂ | Me, | tBu | CH₂OH | AspB | 19.2 | B | 1265.9 | 1.68 | 0.0195 |
| | phenyl | 4- | | ONH₂ | | nBu | | | | | | | | |
| | (4- | OH- | | | | | | | | | | | | |
| | CO | phenyl | | | | | | | | | | | | |
| | NH₂) | | | | | | | | | | | | | |
| 2090 | CH₂- | CH₂- | iBu | CH₂C | guani- | Me, | tBu | CH₂OH | AspB | 31.9 | A | 1299 | 1.42 | 0.0344 |
| | phenyl | indol- | | ONH₂ | dinyl | nBu | | | | | | | | |
| | (4- | 3- | | | | | | | | | | | | |
| | CO | yl | | | | | | | | | | | | |
| | NH₂) | | | | | | | | | | | | | |
| 2091 | CH₂- | CH₂- | iBu | CH₂C | guani- | Me, | tBu | CH₂C | AspB | 25.8 | A | 1313 | 1.38 | 0.0146 |
| | phenyl | indol- | | ONH₂ | dinyl | nBu | | OOH | | | | | | |
| | (4- | 3- | | | | | | | | | | | | |
| | CO | yl | | | | | | | | | | | | |
| | NH₂) | | | | | | | | | | | | | |
| 2092 | CH₂- | CH₂- | iBu | CH₂C | NH₂ | Me, | tBu | CH₂C | AspB | 41.4 | B | 1279.9 | 1.7 | 0.0158 |
| | phenyl | 4- | | ONH₂ | | nBu | | OOH | | | | | | |
| | (4- | OH- | | | | | | | | | | | | |
| | CO | phenyl | | | | | | | | | | | | |
| | NH₂) | | | | | | | | | | | | | |
| 2093 | CH₂- | CH₂- | Me | CH₂C | NH₂ | Me, | tBu | CH₂OH | AspB | 26.2 | A | 1235.1 | 1.52 | 0.0221 |
| | phenyl | indol- | | ONH₂ | | nBu | | | | | | | | |
| | | 3- | | | | | | | | | | | | |
| | | yl | | | | | | | | | | | | |
| 2094 | CH₂- | CH₂- | Me | CH₂C | guani- | Me, | tBu | CH₂C | AspB | 22.7 | B | 1270.2 | 1.78 | 0.0347 |
| | phenyl | indol- | | ONH₂ | dinyl | nBu | | OOH | | | | | | |
| | | 3- | | | | | | | | | | | | |
| | | yl | | | | | | | | | | | | |
| 2095 | CH₂- | CH₂- | Me | CH₂C | NH₂ | Me, | tBu | CH₂C | AspB | 33.5 | A | 1237.1 | 1.41 | 0.0501 |
| | phenyl | 4- | | ONH₂ | | nBu | | OOH | | | | | | |
| | | OH- | | | | | | | | | | | | |
| | | phenyl | | | | | | | | | | | | |
| 2096 | CH₂- | CH₂- | Me | CH₂C | guani- | Me, | tBu | CH₂C | AspB | 21.6 | A | 1258.2 | 1.37 | 0.0200 |
| | phenyl | 4- | | ONH₂ | dinyl | nBu | | OOH | | | | | | |
| | | OH- | | | | | | | | | | | | |
| | | phenyl | | | | | | | | | | | | |
| 2097 | CH₂- | CH₂- | Me | CH₂C | NH₂ | Me, | tBu | CH₂C | AspB | 33.5 | A | 1249.2 | 1.45 | 0.0222 |
| | phenyl | indol- | | ONH₂ | | nBu | | OOH | | | | | | |
| | | 3- | | | | | | | | | | | | |
| | | yl | | | | | | | | | | | | |
| 2098 | CH₂- | CH₂- | Me | CH₂C | guani- | Me, | tBu | CH₂OH | AspB | 23.2 | A | 1256.1 | 1.5 | 0.0366 |
| | phenyl | indol- | | ONH₂ | dinyl | nBu | | | | | | | | |
| | | 3- | | | | | | | | | | | | |
| | | yl | | | | | | | | | | | | |
| 2099 | CH₂- | CH₂- | iBu | CH₂C | NH₂ | Me, | tBu | CH₂OH | AspB | 12 | A | 1259.1 | 1.6 | 0.0369 |
| | phenyl | 4- | | ONH₂ | | nBu | | | | | | | | |
| | | OH- | | | | | | | | | | | | |
| | | phenyl | | | | | | | | | | | | |
| 2100 | CH₂- | CH₂- | iBu | CH₂C | NH₂ | Me, | tBu | CH₂OH | AspB | 5.6 | B | 1252 | 1.86 | 0.0492 |
| | phenyl | 4- | | ONH₂ | | nBu | | | | | | | | |
| | | OH- | | | | | | | | | | | | |
| | | phenyl | | | | | | | | | | | | |
| 2101 | CH₂- | CH₂- | iBu | CH₂C | guani- | Me, | iPr | CH₂OH | AspB | 13.8 | A | 1272 | 1.58 | 0.0474 |
| | phenyl | 4- | | ONH₂ | dinyl | nBu | | | | | | | | |
| | | OH- | | | | | | | | | | | | |
| | | phenyl | | | | | | | | | | | | |

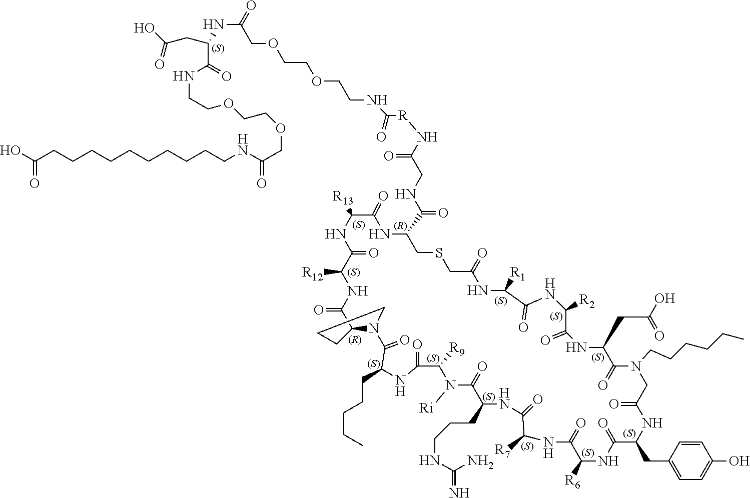

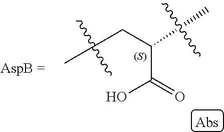

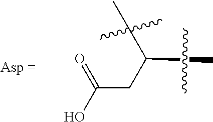

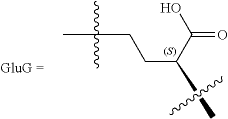

| | | | | | | | | | | | | | HC |
|---|---|---|---|---|---|---|---|---|---|---|---|---|---|
| | | | | | | | | | | | | | CBA |
| | | | | | | | | | | QC | | | mVIS |
| Ex. | | | | | | | | | | Meth- | Obs. | | TA |
| Num- | | | | | Rᵢ, | | | | Yield | hod | MS | | IC50 |
| ber | R₁ | R₂ | R₆ | R₇ | R₉ | R₁₂ | R₁₃ | R | (mg) | IDs | Ion | RT | (uM) |
| 2102 | CH₂- | CH₂- | iBu | CH₂CO | H, | iPr | CH₂OH | AspB | 16.3 | B | 1280.1 | 1.59 | 0.0189 |
| | Phenyl | 4- | | NH₂ | iBu | | | | | | | | |
| | | OH- | | | | | | | | | | | |
| | | Phenyl | | | | | | | | | | | |
| 2103 | CH₂- | CH₂- | iBu | CH₂CO | H, | iPr | iPr | AspB | 3.6 | A | 1284.8 | 1.66 | 0.0067 |
| | Phenyl | Phenyl | | NH₂ | iBu | | | | | | | | |
| | | (3- | | | | | | | | | | | |
| | | Me) | | | | | | | | | | | |
| 2104 | CH₂- | CH₂- | nBu | CH₂CO | H, | iPr | CH₂CO | AspB | 1.9 | A | 1312.2 | 1.47 | 0.0108 |
| | Phenyl | indol- | | NH₂ | iBu | | OH | | | | | | |
| | (3-Me) | 3- | | | | | | | | | | | |
| | | yl | | | | | | | | | | | |
| 2105 | CH₂ | CH₂- | nBu | H | H, | iPr | CH₂CO | AspB | 13.5 | B | 1272 | 1.59 | 0.0498 |
| | Phenyl | 4- | | | iBu | | OH | | | | | | |
| | (3-Me) | OH- | | | | | | | | | | | |
| | | Phenyl | | | | | | | | | | | |
| 2106 | CH₂- | CH₂- | iBu | CH₂CO | H, | iPr | iPr | AspB | 4.2 | B | 1285.2 | 1.57 | 0.0180 |
| | Phenyl | 4- | | NH₂ | iBu | | | | | | | | |
| | | OH- | | | | | | | | | | | |
| | | Phenyl | | | | | | | | | | | |
| 2107 | 2Nal | CH₂- | iPr | H | H, | iPr | CH₂CO | AspB | 9.5 | B | 1298.2 | 1.55 | 0.0389 |
| | | 4- | | | iBu | | OH | | | | | | |
| | | OH- | | | | | | | | | | | |
| | | Phenyl | | | | | | | | | | | |
| 2108 | CH₂- | CH₂- | nBu | CH₂CO | H, | iPr | CH₂CO | AspB | 4.5 | A | 1293.7 | 1.34 | 0.0144 |
| | Phenyl | 4- | | OH | iBu | | NH₂ | | | | | | |
| | | OH- | | | | | | | | | | | |
| | | Phenyl | | | | | | | | | | | |
| 2109 | CH₂- | CH₂- | nBu | CH₂CO | Me, | iPr | CH₂CO | AspB | 23.6 | A | 1337.2 | 1.76 | 0.0274 |
| | 2- | indol- | | NH₂ | nBu | | OH | | | | | | |
| | naphthyl | 3- | | | | | | | | | | | |
| | | yl | | | | | | | | | | | |
| 2110 | CH₂- | CH₂- | nBu | CH₂CO | H, | iPr | CH₂OH | AspB | 5.4 | B | 1304.5 | 1.58 | 0.0216 |
| | 2- | 4- | | NH₂ | iBu | | | | | | | | |
| | naphthyl | OH- | | | | | | | | | | | |
| | | Phenyl | | | | | | | | | | | |
| 2111 | CH₂- | CH₂- | nBu | CH₂CO | Me, | tBu | CH₂OH | AspB | 33.1 | A | 1294.1 | 1.49 | 0.0139 |
| | Phenyl | 4- | | NH₂ | nBu | | | | | | | | |
| | | OH- | | | | | | | | | | | |
| | | Phenyl | | | | | | | | | | | |
| 2112 | CH₂- | CH₂- | nBu | CH₂CO | Me, | tBu | CH₂OH | AspB | 32.1 | B | 870.98 | 1.88 | 0.0183 |
| | Phenyl | indol- | | NH₂ | nBu | | | | | | | | |
| | | 3- | | | | | | | | | | | |
| | | yl | | | | | | | | | | | |
| 2113 | CH₂- | CH₂- | nBu | CH₂CO | Me, | tBu | CH₂OH | AspB | 45.1 | B | 1293.2 | 1.95 | 0.0276 |
| | Phenyl | Phenyl | | NH₂ | nBu | | | | | | | | |
| | | (3- | | | | | | | | | | | |
| | | Me) | | | | | | | | | | | |
| 2114 | CH₂- | CH₂- | nBu | CH₂CO | Me, | tBu | CH₂OH | AspB | 33.4 | A | 1319 | 1.56 | 0.0510 |
| | 2- | 4- | | NH₂ | nBu | | | | | | | | |
| | naphthyl | OH- | | | | | | | | | | | |
| | | Phenyl | | | | | | | | | | | |
| 2115 | CH₂- | CH₂- | nBu | CH₂CO | Me, | tBu | CH₂OH | AspB | 7.8 | B | 875.4 | 1.81 | 0.0373 |
| | Phenyl | indol- | | NH₂ | nBu | | | | | | | | |
| | (3-Me) | 3- | | | | | | | | | | | |
| | | yl | | | | | | | | | | | |
| 2116 | CH₂- | CH₂- | iBu | CH₂CO | H, | iPr | CH₂OH | D | 19.3 | B | 1279.4 | 1.6 | 0.0407 |
| | Phenyl | 4- | | NH₂ | iBu | | | | | | | | |
| | | OH- | | | | | | | | | | | |
| | | Phenyl | | | | | | | | | | | |
| 2117 | CH₂- | CH₂- | iBu | CH₂CO | H, | iPr | CH₂OH | GluG | 32.8 | A | 1286.2 | 1.47 | 0.0335 |
| | Phenyl | 4- | | NH₂ | iBu | | | | | | | | |
| | | OH- | | | | | | | | | | | |
| | | Phenyl | | | | | | | | | | | |
| 2118 | CH₂- | CH₂- | nBu | CH2C | H, | iPr | CH₂CO | GluG | 4.3 | B | 868.1 | 1.69 | 0.0132 |
| | Phenyl | 4- | | OOH | iBu | | NH₂ | | | | | | |
| | | OH- | | | | | | | | | | | |
| | | Phenyl | | | | | | | | | | | |
| 2119 | CH₂- | CH₂- | nBu | CH₂CO | Me, | iPr | CH₂CO | GluG | 4.5 | A | 879.8 | 1.57 | 0.0196 |
| | Phenyl | indol- | | NH₂ | nBu | | OH | | | | | | |
| | | 3- | | | | | | | | | | | |
| | | yl | | | | | | | | | | | |
| 2120 | CH₂- | CH₂- | iPr | H | H, | iPr | CH₂CO | GluG | 6.3 | B | 871.2 | 1.68 | 0.0171 |
| | 2- | 4- | | | iBu | | OH | | | | | | |
| | naphthyl | OH- | | | | | | | | | | | |
| | | Phenyl | | | | | | | | | | | |
| 2121 | CH₂- | CH₂- | nPen- | H | H, | iPr | CH₂OH | GluG | 3.7 | B | 844.1 | 1.9 | 0.0284 |
| | Phenyl | 4- | tyl | | iBu | | | | | | | | |
| | | OH- | | | | | | | | | | | |
| | | Phenyl | | | | | | | | | | | |
| 2122 | CH₂- | CH₂- | iBu | CH₂CO | H, | iPr | iPr | GluG | 4.4 | A | 1293 | 1.54 | 0.0178 |
| | Phenyl | 4- | | NH₂ | iBu | | | | | | | | |
| | | OH- | | | | | | | | | | | |
| | | Phenyl | | | | | | | | | | | |
| 2123 | CH₂- | CH₂- | nBu | CH₂CO | H, | iPr | CH₂OH | GluG | 7.4 | B | 875.2 | 1.75 | 0.0179 |
| | 2- | 4- | | NH₂ | iBu | | | | | | | | |
| | naphthyl | OH- | | | | | | | | | | | |
| | | Phenyl | | | | | | | | | | | |
| 2124 | CH₂- | CH₂- | nBu | H | H, | iPr | CH₂CO | GluG | 2.4 | B | 853.29, | 1.71, | 0.0113 |
| | Phenyl | 4- | | | iBu | | OH | | | | 853.98 | 1.73 | |
| | (3-Me) | OH- | | | | | | | | | | | |
| | | Phenyl | | | | | | | | | | | |
| 2125 | CH₂- | CH₂- | nBu | CH₂CO | Me, | iPr | CH₂CO | GluG | 12.8 | A | 1344.1 | 1.36 | 0.0196 |
| | 2- | indol- | | NH₂ | nBu | | OH | | | | | | |
| | naphthyl | 3- | | | | | | | | | | | |
| | | yl | | | | | | | | | | | |
| 2126 | CH₂- | CH₂- | nBu | CH₂CO | H, | iPr | CH₂CO | GluG | 11.7 | B | 880.1 | 1.76 | 0.0136 |
| | Phenyl | indol- | | NH₂ | iBu | | OH | | | | | | |
| | (3-Me) | 3- | | | | | | | | | | | |
| | | yl | | | | | | | | | | | |

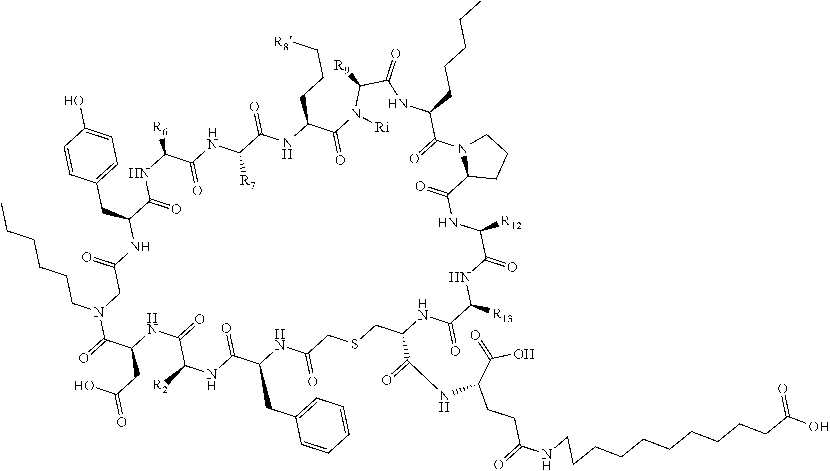

| | | | | | | | | | | | | HC |
|---|---|---|---|---|---|---|---|---|---|---|---|---|
| | | | | | | | | | QC | | | CBA |
| Exam- | | | | | | | | | Meth- | Obs. | | mVISTA |
| ple | | | | | Rᵢ, | | | Yield | od | MS | | IC50 |
| No | R₂ | R₆ | R₇ | R_{8′} | R₉ | R₁₂ | R₁₃ | (mg) | IDs | Ion | RT | (uM) |
| 2127 | CH₂- | iBu | CH₂CO | guani- | H, | iPr | CH₂OH | 13.8 | B | 1055 | 1.75 | 0.0159 |
| | 4- | | NH₂ | dinyl | iBu | | | | | | | |
| | OH- | | | | | | | | | | | |
| | phenyl | | | | | | | | | | | |
| 2128 | CH₂- | iBu | H | guani- | H, | iPr | CH₂OH | 9.4 | A | 1026.9 | 1.67 | 0.0301 |
| | 4- | | | dinyl | iBu | | | | | | | |
| | OH- | | | | | | | | | | | |
| | phenyl | | | | | | | | | | | |
| 2129 | CH₂- | nPen- | CH₂CO | guani- | H, | iPr | CH₂OH | 13.6 | B | 733.8 | 1.79 | 0.0166 |
| | 4- | tyl | NH₂ | dinyl | CH2- | | | | | | | |
| | OH- | | | | indol- | | | | | | | |
| | phenyl | | | | 3yl | | | | | | | |
| 2130 | CH₂- | nBu | H | guani- | H, | iPr | CH₂CO | 16.1 | A | 1041.1 | 1.54 | 0.0372 |
| | 4- | | | dinyl | iBu | | NH₂ | | | | | |
| | OH- | | | | | | | | | | | |
| | phenyl | | | | | | | | | | | |
| 2131 | CH₂- | iBu | CH₂CO | guani- | Me, | tBu | CH₂OH | 44.6 | A | 1081.2 | 1.61 | 0.0380 |
| | indol- | | NH₂ | dinyl | nBu | | | | | | | |
| | 3- | | | | | | | | | | | |
| | yl | | | | | | | | | | | |
| 2132 | CH₂- | iBu | CH₂CO | NH₂ | Me, | tBu | CH₂OH | 54.8 | A | 1049.2 | 1.55 | 0.0235 |
| | 4- | | NH₂ | | nBu | | | | | | | |
| | OH- | | | | | | | | | | | |
| | phenyl | | | | | | | | | | | |
| 2133 | CH₂- | iBu | CH₂CO | guani- | Me, | tBu | CH₂OH | 16.1 | A | 1062.9 | 1.42 | 0.0240 |
| | 4- | | NH₂ | dinyl | iPr | | | | | | | |
| | OH- | | | | | | | | | | | |
| | phenyl | | | | | | | | | | | |
| 2134 | CH₂- | iBu | CH₂CO | NH₂ | Me, | tBu | CH₂OH | 68.7 | A | 1060.9 | 1.65 | 0.0217 |
| | indol- | | NH₂ | | nBu | | | | | | | |
| | 3- | | | | | | | | | | | |
| | yl | | | | | | | | | | | |
| 2135 | CH₂- | iBu | CH₂CO | guani- | Me, | tBu | CH₂OH | 18.5 | B | 1070 | 1.85 | 0.0310 |
| | 4- | | NH₂ | dinyl | iBu | | | | | | | |
| | OH- | | | | | | | | | | | |
| | phenyl | | | | | | | | | | | |
| 2136 | CH₂- | iBu | CH₂CO | guani- | Me, | tBu | CH₂OH | 42.5 | A | 1070.1 | 1.63 | 0.0198 |
| | 4- | | NH₂ | dinyl | nBu | | | | | | | |
| | OH- | | | | | | | | | | | |
| | phenyl | | | | | | | | | | | |
| 2137 | CH₂- | iBu | CH₂CO | NH₂ | Me, | tBu | CH₂OH | 33.8 | A | 1041.5 | 1.48 | 0.0180 |
| | 4- | | NH₂ | | iPr | | | | | | | |
| | OH- | | | | | | | | | | | |
| | phenyl | | | | | | | | | | | |
| 2138 | CH₂- | iBu | CH₂CO | NH₂ | Me, | tBu | CH₂OH | 81.7 | B | 1049 | 1.89 | 0.0330 |
| | 4- | | NH₂ | | iBu | | | | | | | |
| | OH- | | | | | | | | | | | |
| | phenyl | | | | | | | | | | | |
| 2139 | CH₂- | (CH2)4 | CH₂CO | NH₂ | Me, | tBu | CH₂OH | 17.4 | B | 1057.1 | 1.64 | 0.0457 |
| | 4- | NH2 | NH₂ | | nBu | | | | | | | |
| | OH- | | | | | | | | | | | |
| | phenyl | | | | | | | | | | | |
| 2140 | CH₂- | (CH₂)₄ | CH₂CO | NH₂ | Me, | tBu | CH₂CO | 22.1 | A | 1070.5 | 1.45 | 0.0419 |
| | 4- | NH₂ | NH₂ | | nBu | | OH | | | | | |
| | OH- | | | | | | | | | | | |
| | phenyl | | | | | | | | | | | |
| 2141 | CH₂- | nPen- | CH₂CO | NH₂ | Me, | tBu | CH₂CO | 15.9 | A | 1070.2 | 1.55 | 0.0265 |
| | 4- | tyl | NH₂ | | nBu | | OH | | | | | |
| | OH- | | | | | | | | | | | |
| | phenyl | | | | | | | | | | | |
| 2142 | CH₂- | nPen- | CH₂CO | guani- | Me, | tBu | CH₂OH | 12.8 | A | 1077.1 | 1.63 | 0.0325 |
| | 4- | tyl | NH₂ | dinyl | nBu | | | | | | | |
| | OH- | | | | | | | | | | | |
| | phenyl | | | | | | | | | | | |
| 2143 | CH₂- | nPen- | CH₂CO | NH₂ | Me, | tBu | CH₂OH | 17.5 | A | 1056 | 1.66 | 0.0294 |
| | 4- | tyl | NH₂ | | nBu | | | | | | | |
| | OH- | | | | | | | | | | | |
| | phenyl | | | | | | | | | | | |
| 2144 | CH₂- | nPen- | CH₂CO | NH₂ | Me, | tBu | CH₂OH | 13.9 | B | 1067.99 | 2 | 0.0390 |
| | indol- | tyl | NH₂ | | nBu | | | | | | | |
| | 3- | | | | | | | | | | | |
| | yl | | | | | | | | | | | |
| 2340 | CH₂- | nPen- | CH₂CO | guani- | H, | iPr | CH₂OH | 10.3 | A | 1063.3 | 1.83 | 0.019 |
| | 4- | tyl | NH₂ | dinyl | iBu | | | | | | | |
| | OH- | | | | | | | | | | | |
| | phenyl | | | | | | | | | | | |

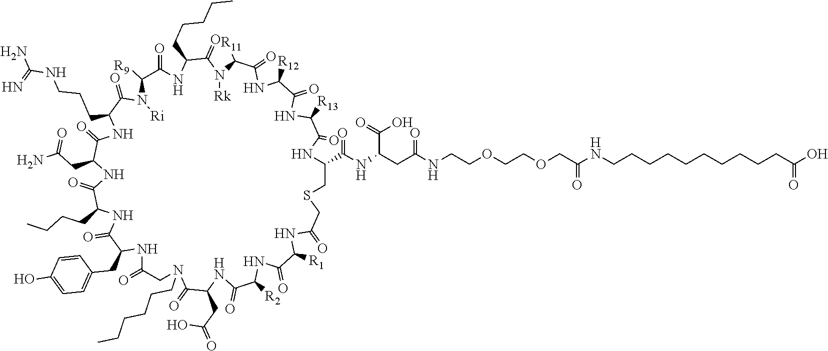

| | | | | | | | | | | | HC |
|---|---|---|---|---|---|---|---|---|---|---|---|
| Exam- | | | | | | | | | | | CBA |
| ple | | | | | | | | QC | Obs. | | mVISTA |
| Num- | | | | | | | Yield | Method | MS | | IC50 |
| ber | R₁ | R₂ | Rᵢ,R₉ | Rₖ, R₁₁ | R₁₂ | R₁₃ | (mg) | IDs | Ion | RT | u(M) |
| 2145 | CH₂- | CH₂- | Tic | Pro | iBu | CH₂CO | 32.7 | B | 1183.3 | 1.79 | 0.0168 |
| | 2- | 4-OH- | | | | OH | | | | | |
| | naph- | phenyl | | | | | | | | | |
| | thyl | | | | | | | | | | |
| 2146 | CH₂- | CH₂- | Me, | Pro | iBu | CH₂CO | 30.2 | A | 1154 | 1.42 | 0.0127 |
| | phenyl | indol- | nBu | | | OH | | | | | |
| | | 3-yl | | | | | | | | | |
| 2147 | CH₂- | CH₂- | Me | Pro | iBu | CH₂CO | 6.4 | B | 1168 | 1.84 | 0.0152 |
| | 2- | 4-OH- | nBu | | | OH | | | | | |
| | naph- | phenyl | | | | | | | | | |
| | thyl | | | | | | | | | | |
| 2148 | CH₂- | CH₂- | Tic | Pro | iBu | CH₂CO | 21.5 | A | 1170.1 | 1.41 | 0.0367 |
| | phenyl | indol- | | | | OH | | | | | |
| | | 3-yl | | | | | | | | | |
| 2149 | CH₂- | CH₂- | Me, | Pro | iBu | CH₂CO | 33.7 | B | 1154.4 | 1.82 | 0.0273 |
| | phenyl | indol- | nBu | | | OH | | | | | |
| | | 3-yl | | | | | | | | | |
| 2150 | CH₂- | CH₂- | Me, iBu | Pro | iBu | CH₂CO | 13.3 | A | 1167.9 | 1.51 | 0.0107 |
| | 2- | 4-OH- | | | | OH | | | | | |
| | naph- | phenyl | | | | | | | | | |
| | thyl | | | | | | | | | | |
| 2151 | CH₂- | CH₂- | Me, | Pro | iBu | CH₂CO | 16.5 | A | 1167.2 | 1.6 | 0.0108 |
| | 2- | 4-OH- | nBu | | | OH | | | | | |
| | naph- | phenyl | | | | | | | | | |
| | thyl | | | | | | | | | | |
| 2152 | CH₂- | CH₂- | Tic | Pro | iBu | CH₂CO | 8.5 | A | 1184.2 | 1.55 | 0.0222 |
| | 2- | 4-OH- | | | | OH | | | | | |
| | naph- | phenyl | | | | | | | | | |
| | thyl | | | | | | | | | | |
| 2153 | CH₂- | CH₂- | Tic | Pro | iBu | CH₂CO | 14.4 | B | 1170.1 | 1.81 | 0.0129 |
| | phenyl | indol- | | | | OH | | | | | |
| | | 3-yl | | | | | | | | | |
| 2154 | CH₂- | CH₂- | Me, iBu | Pro | iBu | CH₂CO | 24.2 | A | 769.96, | 1.38, | 0.0334 |
| | phenyl | indol- | | | | OH | | | 1155.16 | 1.51 | |
| | | 3-yl | | | | | | | | | |
| 2155 | CH₂- | CH₂- | Me, iBu | Pro | iBu | CH₂CO | 6.7 | B | 1153.9 | 1.84 | 0.0171 |
| | phenyl | indol- | | | | OH | | | | | |
| | | 3-yl | | | | | | | | | |
| 2156 | CH₂- | CH₂- | Me, iBu | Pro | iBu | CH₂CO | 10 | A | 1167.1 | 1.45 | 0.0256 |
| | 2- | 4-OH- | | | | OH | | | | | |
| | naph- | phenyl | | | | | | | | | |
| | thyl | | | | | | | | | | |
| 2157 | CH₂- | CH₂- | Tic | Pro | iBu | CH₂OH | 12.5 | B | 1156.2 | 1.91 | 0.0105 |
| | phenyl | indol- | | | | | | | | | |
| | | 3-yl | | | | | | | | | |
| 2158 | CH₂- | CH₂- | Me, iBu | Pro | iBu | CH₂OH | 8.4 | A | 1140 | 1.68 | 0.0123 |
| | phenyl | indol- | | | | | | | | | |
| | | 3-yl | | | | | | | | | |
| 2159 | CH₂- | CH₂- | Me, | Pro | iBu | CH₂OH | 30.4 | A | 1153.3 | 1.49 | 0.0255 |
| | 2- | 4-OH- | nBu | | | | | | | | |
| | naph- | phenyl | | | | | | | | | |
| | thyl | | | | | | | | | | |
| 2160 | CH₂- | CH₂- | Tic | Pro | iBu | CH₂OH | 30.8 | A | 1170 | 1.45 | 0.0378 |
| | 2- | 4-OH- | | | | | | | | | |
| | naph- | phenyl | | | | | | | | | |
| | thyl | | | | | | | | | | |
| 2161 | CH₂- | CH₂- | Me, | Pro | iBu | CH₂OH | 31.5 | A | 1140.2 | 1.5 | 0.0299 |
| | phenyl | indol- | nBu | | | | | | | | |
| | | 3-yl | | | | | | | | | |
| 2162 | CH₂- | CH₂- | Me, iBu | Pro | iBu | CH₂OH | 10.4 | A | 1153.3 | 1.51 | 0.0190 |
| | 2- | 4-OH- | | | | | | | | | |
| | naph- | phenyl | | | | | | | | | |
| | thyl | | | | | | | | | | |
| 2163 | CH₂- | CH₂- | Me, iBu | Pro | iBu | CH₂OH | 9.7 | A | 1140.2 | 1.42 | 0.0140 |
| | phenyl | indol- | | | | | | | | | |
| | | 3-yl | | | | | | | | | |
| 2164 | CH₂- | CH₂- | Me, iBu | Pro | iBu | CH₂OH | 25.2 | B | 1154 | 1.77 | 0.0355 |
| | 2- | 4-OH- | | | | | | | | | |
| | naph- | phenyl | | | | | | | | | |
| | thyl | | | | | | | | | | |
| 2165 | CH₂- | CH₂- | Me, | Pro | iBu | CH₂OH | 24.1 | A | 760.35 | 1.43 | 0.0167 |
| | phenyl | indol- | nBu | | | | | | | | |
| | | 3-yl | | | | | | | | | |
| 2166 | CH₂- | CH₂- | Tic | Pro | iBu | CH₂OH | 23.2 | A | 1155.9 | 1.51 | 0.0097 |
| | phenyl | indol- | | | | | | | | | |
| | | 3-yl | | | | | | | | | |
| 2167 | CH₂- | CH₂- | Me, | Pro | iBu | CH₂OH | 26.1 | A | 1154 | 1.4 | 0.0249 |
| | 2- | 4-OH- | nBu | | | | | | | | |
| | naph- | phenyl | | | | | | | | | |
| | thyl | | | | | | | | | | |
| 2168 | CH₂- | CH₂- | Tic | Pro | iBu | CH₂OH | 17.2 | A | 1169.1 | 1.54 | 0.0180 |
| | 2- | 4-OH- | | | | | | | | | |
| | naph- | phenyl | | | | | | | | | |
| | thyl | | | | | | | | | | |
| 2169 | CH₂- | CH₂- | Me, | Me, Me | tBu | CH₂OH | 12.1 | B | 1140.8 | 1.95 | 0.0198 |
| | phenyl | indol- | nBu | | | | | | | | |
| | | 3-yl | | | | | | | | | |
| 2170 | CH₂- | CH₂- | Me, | Me, nBu | iBu | CH₂OH | 2.3 | A | 1154.8 | 1.68 | 0.0212 |
| | phenyl | indol- | nBu | | | | | | | | |
| | | 3-yl | | | | | | | | | |
| 2171 | CH₂- | CH₂- | Me, | Me, Me | CHMe | CH₂OH | 6.5 | A | 1140.9 | 1.8 | 0.0288 |
| | phenyl | indol- | nBu | | Et | | | | | | |
| | | 3-yl | | | | | | | | | |
| 2172 | CH₂- | CH₂- | Me, | Me, Me | iPr | CH₂OH | 9.9 | A | 1134.2 | 1.59 | 0.0306 |
| | phenyl | indol- | nBu | | | | | | | | |
| | | 3-yl | | | | | | | | | |
| 2173 | CH₂- | CH₂- | Me, | Me, nBu | tBu | CH₂OH | 6.2 | B | 1162.1 | 1.96 | 0.0351 |
| | phenyl | indol- | nBu | | | | | | | | |
| | | 3-yl | | | | | | | | | |
| 2174 | CH₂- | CH₂- | Me, Me | Pro | iPr | CH₂OH | 13.5 | A | 1119 | 1.47 | 0.0166 |
| | phenyl | indol- | | | | | | | | | |
| | | 3-yl | | | | | | | | | |
| 2175 | CH₂- | CH₂- | Y(CCO | Pro | iPr | CH₂OH | 22 | A | 1187.2 | 1.58 | 0.0354 |
| | phenyl | indol- | OH) | | | | | | | | |
| | | 3-yl | | | | | | | | | |
| 2176 | CH₂- | CH₂- | F(4- | Pro | iPr | CH₂OH | 20 | A | 1172 | 1.58 | 0.0198 |
| | phenyl | indol- | COOH) | | | | | | | | |
| | | 3-yl | | | | | | | | | |
| 2177 | CH₂- | CH₂- | Me, | Pro | iPr | CH₂OH | 11.1 | A | 1157 | 1.54 | 0.0384 |
| | phenyl | indol- | CH2 | | | | | | | | |
| | | 3-yl | Phenyl | | | | | | | | |
| 2178 | CH₂- | CH₂- | Me, | Me, iBu | CHMe | CH₂OH | 10.6 | A | 1162.1 | 1.57 | 0.0467 |
| | phenyl | indol- | nBu | | Et | | | | | | |
| | | 3-yl | | | | | | | | | |
| 2179 | CH₂- | CH₂- | Me, | Me, iBu | tBu | CH₂OH | 40 | B | 775.1 | 1.9 | 0.0302 |
| | phenyl | indol- | nBu | | | | | | | | |
| | | 3-yl | | | | | | | | | |
| 2180 | CH₂- | CH₂- | Me, | Me, iBu | iPr | CH₂OH | 13.3 | A | 1154.6 | 1.74 | 0.0185 |
| | phenyl | indol- | nBu | | | | | | | | |
| | | 3-yl | | | | | | | | | |
| 2181 | CH₂- | CH₂- | Me, | Piperi- | tBu | CH₂OH | 12.4 | A | 1154.2 | 1.73 | 0.0400 |
| | phenyl | indol- | nBu | dinyl | | | | | | | |
| | | 3-yl | | | | | | | | | |
| 2182 | CH₂- | CH₂- | Me, | Piperi- | CHMe | CH₂OH | 7.8 | B | 1154.1 | 1.71 | 0.0309 |
| | phenyl | indol- | nBu | dinyl | Et | | | | | | |
| | | 3-yl | | | | | | | | | |
| 2183 | CH₂- | CH₂- | Me, | Azetidin | CHMe | CH₂OH | 17.6 | A | 1140 | 1.48 | 0.0169 |
| | phenyl | indol- | nBu | yl | Et | | | | | | |
| | | 3-yl | | | | | | | | | |
| 2184 | CH₂- | CH₂- | Me, | Azetidin | iPr | CH₂OH | 15.8 | A | 1133.2 | 1.75 | 0.0231 |
| | phenyl | indol- | nBu | yl | | | | | | | |
| | | 3-yl | | | | | | | | | |
| 2185 | CH₂- | CH₂- | Me, | Piperidi | CH2 | CH₂OH | 11.9 | B | 1171 | 2.1 | 0.0501 |
| | phenyl | indol- | nBu | nyl | Phenyl | | | | | | |
| | | 3-yl | | | | | | | | | |
| 2186 | CH₂- | CH₂- | Me, | Piperidi | iPr | CH₂OH | 15.9 | B | 1147.1 | 1.95, | 0.0244 |
| | phenyl | indol- | nBu | nyl | | | | | | 1.98 | |
| | | 3-yl | | | | | | | | | |
| 2187 | CH₂- | CH₂- | Me, | Azetidin | tBu | CH₂OH | 25.7 | B | 1139.9 | 1.91 | 0.0133 |
| | phenyl | indol- | nBu | yl | | | | | | | |
| | | 3-yl | | | | | | | | | |
| 2188 | CH₂- | CH₂- | Me, | Azetidin | CH2 | CH₂OH | 35.5 | A | 1157.2 | 1.7 | 0.0473 |
| | phenyl | indol- | nBu | yl | Phenyl | | | | | | |
| | | 3-yl | | | | | | | | | |
| 2189 | CH₂- | CH₂- | Me, | Pro | tBu | CH₂OH | 20.6 | A | 1134.3 | 1.75 | 0.0290 |
| | phenyl | phenyl | nBu | | | | | | | | |
| | | (3- | | | | | | | | | |
| | | Me) | | | | | | | | | |
| 2190 | CH₂- | CH₂- | Me, | Pro | tBu | CH₂OH | 32.3 | B | 1135.1 | 1.88 | 0.0244 |
| | phenyl | 4-OH- | nBu | | | | | | | | |
| | | phenyl | | | | | | | | | |
| 2191 | CH₂- | CH₂- | Me, | Pro | tBu | CH₂OH | 28.2 | A | 1147 | 1.66 | 0.0197 |
| | phenyl | indol- | nBu | | | | | | | | |
| | | 3-yl | | | | | | | | | |
| 2192 | CH₂- | CH₂- | Me, | Morpho- | tBu | CH₂OH | 32.8 | A | 1155.1 | 1.62 | 0.0186 |
| | phenyl | indol- | nBu | linyl | | | | | | | |
| | | 3-yl | | | | | | | | | |
| 2193 | CH₂- | CH₂- | Me, | Morpho- | iPr | CH₂OH | 25.1 | B | 1147.9 | 1.9 | 0.0161 |
| | phenyl | indol- | nBu | linyl | | | | | | | |
| | | 3-yl | | | | | | | | | |
| 2194 | CH₂- | CH₂- | Me, | Pro(4-F) | tBu | CH₂OH | 39.4 | B | 1156.2 | 1.96 | 0.0386 |
| | phenyl | indol- | nBu | | | | | | | | |
| | | 3-yl | | | | | | | | | |
| 2195 | CH₂- | CH₂- | Me, | Pro | tBu | CH₂OH | 22.2 | B | 1161 | 1.93 | 0.0303 |
| | 2- | 4-OH- | nBu | | | | | | | | |
| | naph- | phenyl | | | | | | | | | |
| | thyl | | | | | | | | | | |
| 2196 | CH₂- | CH₂- | Me, | Pro(4-F) | iPr | CH₂OH | 17.8 | A | 1148.9 | 1.63 | 0.0284 |
| | phenyl | indol- | nBu | | | | | | | | |
| | | 3-yl | | | | | | | | | |
| 2197 | CH₂- | CH₂- | Me, | Pro | tBu | CH₂OH | 7.3 | A | 1154 | 1.67 | 0.0326 |
| | phenyl | indol- | nBu | | | | | | | | |
| | (3-Me) | 3-yl | | | | | | | | | |

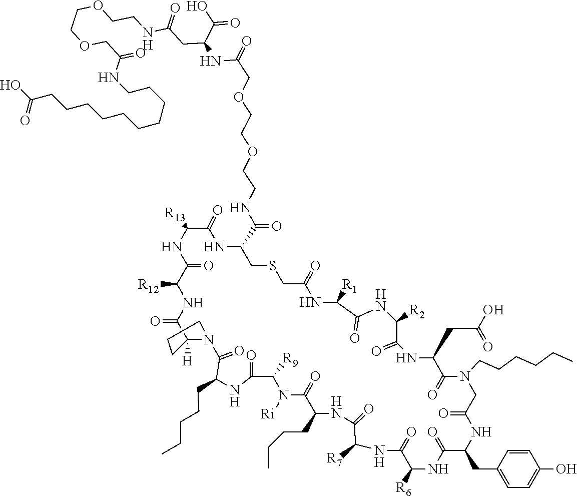

| | | | | | | | | | | | | | HC |
|---|---|---|---|---|---|---|---|---|---|---|---|---|---|
| Exam- | | | | | | | | | | QC | | | CBA |
| ple | | | | | | | | | | Meth- | Obs. | | mVISTA |
| Num- | | | | | | Rᵢ, | | | Yield | od | MS | | IC50 |
| ber | R₁ | R₂ | R₆ | R₇ | R_{8′} | R₉ | R₁₂ | R₁₃ | (mg) | IDs | Ion | RT | (uM) |
| 2198 | CH₂- | CH₂- | nBu | CH₂CO | guani- | H, | iPr | CH₂CO | 5.4 | A | 1226.8 | 1.57 | 0.0152 |
| | phenyl | indol- | | NH₂ | dinyl | iBu | | OH | | | | | |
| | (3- | 3-yl | | | | | | | | | | | |
| | Me) | | | | | | | | | | | | |
| 2199 | CH₂- | CH₂- | npen- | H | guani- | H, | iPr | CH₂OH | 3.3 | A | 782 | 1.59 | 0.0183 |
| | phenyl | 4-OH- | tyl | | dinyl | iBu | | | | | | | |
| | | phenyl | | | | | | | | | | | |
| 2200 | CH₂- | CH₂- | nBu | CH₂CO | guani- | Me, | iPr | CH₂CO | 17.5 | B | 1251.4 | 1.82 | 0.0165 |
| | 2- | indol- | | NH₂ | dinyl | nBu | | OH | | | | | |
| | naph- | 3-yl | | | | | | | | | | | |
| | thyl | | | | | | | | | | | | |
| 2201 | CH₂- | CH₂- | iBu | CH₂CO | guani- | H, | iPr | iPr | 13.6 | A | 1201 | 1.55 | 0.0209 |
| | phenyl | 4-OH- | | NH₂ | dinyl | iBu | | | | | | | |
| | | phenyl | | | | | | | | | | | |
| 2202 | CH₂- | CH₂- | nBu | CH₂CO | guani- | H, | iPr | CH₂CO | 5.4 | A | 1208.3 | 1.44 | 0.0095 |
| | phenyl | 4-OH- | | OH | dinyl | iBu | | NH₂ | | | | | |
| | | phenyl | | | | | | | | | | | |
| 2203 | CH₂- | CH₂- | nBu | H | guani- | H, | iPr | CH₂CO | 8.2 | A | 1186.4 | 1.53 | 0.0215 |
| | phenyl | 4-OH- | | | dinyl | iBu | | OH | | | | | |
| | (3- | phenyl | | | | | | | | | | | |
| | Me) | | | | | | | | | | | | |
| 2204 | CH₂- | CH₂- | nBu | CH₂CO | guani- | Me, | iPr | CH₂CO | 10.7 | B | 818.3 | 1.78 | 0.0149 |
| | phenyl | indol- | | NH₂ | dinyl | nBu | | OH | | | | | |
| | | 3-yl | | | | | | | | | | | |
| 2205 | CH₂- | CH₂- | iPr | H | guani- | H, | iPr | CH₂CO | 24.3 | A | 1198 | 1.43 | 0.0260 |
| | 2- | 4-OH- | | | dinyl | iBu | | OH | | | | | |
| | naph- | phenyl | | | | | | | | | | | |
| | thyl | | | | | | | | | | | | |
| 2206 | CH₂- | CH₂- | nBu | CH₂CO | guani- | H, | iPr | CH₂OH | 5.7 | A | 1219 | 1.56 | 0.0163 |
| | 2- | 4-OH- | | NH₂ | dinyl | iBu | | | | | | | |
| | naph- | phenyl | | | | | | | | | | | |
| | thyl | | | | | | | | | | | | |
| 2207 | CH₂- | CH₂- | iBu | CH₂CO | guani- | H, | iPr | iPr | 5.8 | B | 1199.1 | 1.99 | 0.0507 |
| | phenyl | phenyl | | NH₂ | dinyl | iBu | | | | | | | |
| | | (3- | | | | | | | | | | | |
| | | Me) | | | | | | | | | | | |
| 2208 | CH₂- | CH₂- | iBu | CH₂CO | NH₂ | Me, | tBu | CH₂CO | 59.7 | B | 1200.2 | 2 | 0.0245 |
| | phenyl | phenyl | | OH | | nBu | | NH₂ | | | | | |
| | | (3- | | | | | | | | | | | |
| | | Me) | | | | | | | | | | | |
| 2209 | CH₂- | CH₂- | iBu | CH₂CO | NH₂ | Me, | tBu | CH₂CO | 52.2 | A | 1201.3 | 1.42 | 0.0252 |
| | phenyl | 4-OH- | | OH | | nBu | | NH₂ | | | | | |
| | | phenyl | | | | | | | | | | | |
| 2210 | CH₂- | CH₂- | iBu | CH₂CO | NH₂ | Me, | tBu | CH₂CO | 62.4 | A | 1212.1 | 1.52 | 0.0194 |
| | phenyl | indol- | | OH | | nBu | | NH₂ | | | | | |
| | | 3-yl | | | | | | | | | | | |
| 2211 | CH₂- | CH₂- | iBu | CH₂CO | guani- | Me, | tBu | CH₂CO | 40.1 | A | 1234 | 1.6 | 0.0211 |
| | phenyl | indol- | | OH | dinyl | nBu | | NH₂ | | | | | |
| | | 3-yl | | | | | | | | | | | |
| 2212 | CH₂- | CH₂- | iBu | CH₂CO | guani- | Me, | tBu | CH₂CO | 65.9 | B | 1221.1 | 1.99 | 0.0349 |
| | phenyl | phenyl | | OH | dinyl | nBu | | NH₂ | | | | | |
| | | (3- | | | | | | | | | | | |
| | | Me) | | | | | | | | | | | |
| 2213 | CH₂- | CH₂- | iBu | CH₂CO | guani- | Me, | tBu | CH₂CO | 32.6 | B | 1222.2 | 1.85 | 0.0328 |
| | phenyl | 4-OH- | | OH | dinyl | nBu | | NH₂ | | | | | |
| | | phenyl | | | | | | | | | | | |

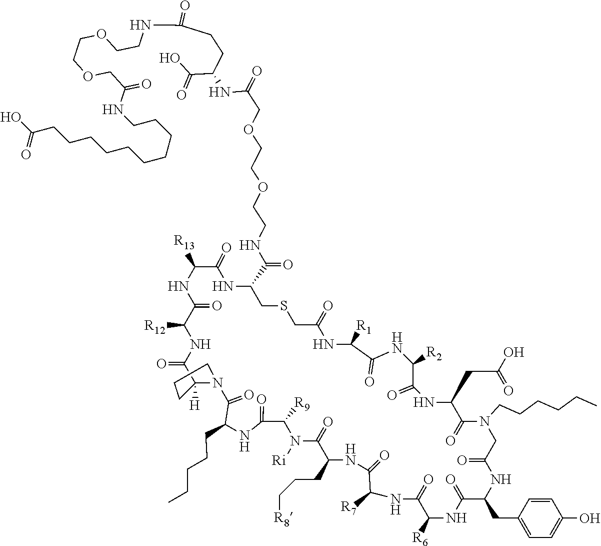

| | | | | | | | | | | | | | HC |
|---|---|---|---|---|---|---|---|---|---|---|---|---|---|
| | | | | | | | | | | QC | | | CBA |
| | | | | | | | | | | Meth- | Obs. | | mVISTA |
| Ex. | | | | | | | | | Yield | od | MS | | IC50 |
| No. | R₁ | R₂ | R₆ | R₇ | R_{8′} | Rᵢ, R₉ | R₁₂ | R₁₃ | (mg) | IDs | Ion | RT | (uM) |
| 2214 | CH₂- | CH₂- | nBu | CH₂C | guani- | Me, | iPr | CH₂C | 20.2 | B | 1259 | 1.81 | 0.0327 |
| | 2- | indol- | | ONH₂ | dinyl | nBu | | OOH | | | | | |
| | naph- | 3-yl | | | | | | | | | | | |
| | thyl | | | | | | | | | | | | |
| 2215 | CH₂- | CH₂-4- | nPen- | H | guani- | H, iBu | iPr | CH₂O | 7.1 | A | 1180 | 1.59 | 0.0283 |
| | phenyl | OH- | tyl | | dinyl | | | H | | | | | |
| | | phenyl | | | | | | | | | | | |
| 2216 | CH₂- | CH₂-4- | nBu | H | guani- | H, iBu | iPr | CH₂C | 5.6 | B | 796.2 | 1.77 | 0.0243 |
| | phenyl | OH- | | | dinyl | | | OOH | | | | | |
| | (3- | phenyl | | | | | | | | | | | |
| | Me) | | | | | | | | | | | | |
| 2217 | CH₂- | CH₂-4- | iBu | CH₂C | guani- | H, iBu | iPr | iPr | 6.7 | A | 1207.3 | 1.63 | 0.0131 |
| | pheny | OH- | | ONH₂ | dinyl | | | | | | | | |
| | | phenyl | | | | | | | | | | | |
| 2218 | CH₂- | CH₂- | nBu | CH₂C | guani- | Me, | iPr | CH₂C | 13.6 | B | 1234 | 1.78 | 0.0115 |
| | pheny | indol- | | ONH₂ | dinyl | nBu | | OOH | | | | | |
| | | 3-yl | | | | | | | | | | | |
| 2219 | CH₂- | CH₂-4- | V | H | guani- | H, iBu | iPr | CH₂C | 13.1 | A | 1205 | 1.43 | 0.0240 |
| | 2- | OH- | | | dinyl | | | OOH | | | | | |
| | naph- | phenyl | | | | | | | | | | | |
| | thyl | | | | | | | | | | | | |
| 2220 | CH₂- | CH₂-4- | nBu | CH₂C | guani- | H, iBu | iPr | N | 4.3 | A | 1215.4 | 1.44 | 0.0199 |
| | pheny | OH- | | OOH | dinyl | | | | | | | | |
| | | phenyl | | | | | | | | | | | |
| 2221 | CH₂- | CH₂ | iBu | CH₂C | guani- | H, iBu | iPr | iPr | 6.3 | A | 1206.1 | 1.78 | 0.0335 |
| | pheny | phenyl | | ONH₂ | dinyl | | | | | | | | |
| | yl | (3-Me) | | | | | | | | | | | |
| 2222 | CH₂- | CH₂- | nBu | CH₂C | guani- | H, iBu | iPr | CH₂C | 8.7 | B | 8231 | 1.91 | 0.0287 |
| | phenyl | indol- | | ONH₂ | dinyl | | | OOH | | | | | |
| | (3- | 3-yl | | | | | | | | | | | |
| | Me) | | | | | | | | | | | | |
| 2223 | CH₂- | CH₂-4- | nBu | CH₂C | guani- | H, iBu | iPr | CH₂O | 23.5 | B | 818 | 1.8 | 0.0124 |
| | 2- | OH- | | ONH₂ | dinyl | | | H | | | | | |
| | naph- | phenyl | | | | | | | | | | | |
| | thyl | | | | | | | | | | | | |
| 2224 | CH₂- | CH₂-4- | nBu | CH₂C | guani- | Me, | tBu | CH₂O | 30.6 | B | 1215 | 1.85 | 0.0186 |
| | pheny | OH- | | ONH₂ | dinyl | nBu | | H | | | | | |
| | | phenyl | | | | | | | | | | | |
| 2225 | CH₂- | CH₂ | nBu | CH₂C | guani- | Me, | tBu | CH₂O | 38 | A | 1214 | 1.74 | 0.0308 |
| | pheny | phenyl | | ONH₂ | dinyl | nBu | | H | | | | | |
| | | (3-Me) | | | | | | | | | | | |
| 2226 | CH₂- | CH₂- | nBu | CH₂C | guani- | Me, | tBu | CH₂O | 28.8 | B | 818 | 1.94 | 0.0244 |
| | pheny | indol- | | ONH₂ | dinyl | nBu | | H | | | | | |
| | | 3-yl | | | | | | | | | | | |
| 2227 | CH₂- | CH₂-4- | nBu | CH₂C | guani- | Me, | tBu | CH₂O | 41.1 | A | 1240 | 1.67 | 0.0415 |
| | 2- | OH- | | ONH₂ | dinyl | nBu | | H | | | | | |
| | naph- | phenyl | | | | | | | | | | | |
| | thyl | | | | | | | | | | | | |
| 2228 | CH₂- | CH₂- | nBu | CH₂C | guani- | Me, | tBu | CH₂O | 20.6 | A | 1234.1 | 1.67 | 0.0388 |
| | phenyl | indol- | | ONH₂ | dinyl | nBu | | H | | | | | |
| | (3- | 3-yl | | | | | | | | | | | |
| | Me) | | | | | | | | | | | | |
| 2229 | CH₂- | CH₂- | nBu | CH₂C | guani- | Me, | tBu | CH₂O | 41.4 | A | 1227 | 1.62 | 0.0331 |
| | pheny | indol- | | ONH₂ | dinyl | iBu | | H | | | | | |
| | | 3-yl | | | | | | | | | | | |
| 2230 | CH₂- | CH₂- | iBu | CH₂C | guani- | H, iBu | tBu | CH₂O | 25.8 | A | 1220 | 1.69 | 0.0284 |
| | pheny | indol- | | ONH₂ | dinyl | | | H | | | | | |
| | | 3-yl | | | | | | | | | | | |
| 2231 | CH₂- | CH₂- | iBu | CH₂C | guani- | H, iBu | tBu | iPr | 30.6 | A | 8171 | 1.74 | 0.0425 |
| | pheny | indol- | | ONH₂ | dinyl | | | | | | | | |
| | | 3-yl | | | | | | | | | | | |
| 2232 | CH₂- | CH₂-4- | iBu | CH₂C | guani- | H, iBu | tBu | CH₂C | 33.1 | A | 1222.1 | 1.51 | 0.0130 |
| | pheny | OH- | | ONH₂ | dinyl | | | OOH | | | | | |
| | | phenyl | | | | | | | | | | | |
| 2233 | CH₂- | CH₂-4- | nBu | CH₂C | guani- | H, iBu | tBu | CH₂C | 41.2 | A | 1222 | 1.52 | 0.0291 |
| | pheny | OH- | | ONH₂ | dinyl | | | OOH | | | | | |
| | | phenyl | | | | | | | | | | | |
| 2234 | CH₂- | CH₂-4- | nBu | CH₂C | guani- | H, iBu | tBu | iPr | 25.4 | A | 1214.1 | 1.67 | 0.0322 |
| | pheny | OH- | | ONH₂ | dinyl | | | | | | | | |
| | | phenyl | | | | | | | | | | | |
| 2235 | CH₂- | CH₂-4- | iBu | CH₂C | guani- | H, iBu | tBu | CH₂O | 31 | A | 1208.2 | 1.57 | 0.0151 |
| | pheny | OH- | | ONH₂ | dinyl | | | H | | | | | |
| | | phenyl | | | | | | | | | | | |
| 2236 | CH₂- | CH₂- | iBu | CH₂C | guani- | H, iBu | tBu | CH₂C | 33 | A | 1234 | 1.58 | 0.0237 |
| | pheny | indol- | | ONH₂ | dinyl | | | OOH | | | | | |
| | | 3-yl | | | | | | | | | | | |
| 2237 | CH₂- | CH₂- | nBu | CH₂C | guani- | H, iBu | tBu | CH₂C | 45.6 | A | 1234 | 1.58 | 0.0362 |
| | pheny | indol- | | ONH₂ | dinyl | | | OOH | | | | | |
| | | 3-yl | | | | | | | | | | | |
| 2238 | CH₂- | CH₂-4- | nBu | CH₂C | guani- | H, iBu | tBu | CH₂O | 13.9 | A | 1208.1 | 1.69 | 0.0380 |
| | pheny | OH- | | ONH₂ | dinyl | | | H | | | | | |
| | | phenyl | | | | | | | | | | | |
| 2239 | CH₂- | CH₂- | nBu | CH₂C | guani- | Me, | tBu | iPr | 26.9 | A | 1233.3 | 1.79 | 0.0486 |
| | pheny | indol- | | ONH₂ | dinyl | nBu | | | | | | | |
| | | 3-yl | | | | | | | | | | | |
| 2240 | CH₂- | CH₂-4- | iBu | CH₂C | guani- | H, iBu | tBu | iPr | 23.9 | A | 1214.1 | 1.67 | 0.0244 |
| | pheny | OH- | | ONH₂ | dinyl | | | | | | | | |
| | | phenyl | | | | | | | | | | | |
| 2241 | CH₂- | CH₂- | nBu | CH₂C | guani- | H, iPr | tBu | CH₂O | 16.1 | B | 1217.1 | 1.9 | 0.0295 |
| | pheny | indol- | | ONH₂ | dinyl | | | H | | | | | |
| | | 3-yl | | | | | | | | | | | |
| 2242 | CH₂- | CH₂- | nBu | CH₂C | guani- | Me, | tBu | CH₂O | 27.2 | A | 828.1 | 1.57 | 0.0197 |
| | pheny | indol- | | ONH₂ | dinyl | nBu | | H | | | | | |
| | | 3-yl | | | | | | | | | | | |
| 2243 | CH₂- | CH₂- | nBu | CH₂C | guani- | H, iBu | tBu | CH₂O | 40.4 | A | 1220.2 | 1.78 | 0.0389 |
| | pheny | indol- | | ONH₂ | dinyl | | | H | | | | | |
| | | 3-yl | | | | | | | | | | | |
| 2244 | CH₂- | CH₂- | nBu | CH₂C | guani- | Tic | tBu | CH₂O | 38.7 | A | 1243.1 | 1.6 | 0.0322 |
| | pheny | indol- | | ONH₂ | dinyl | | | H | | | | | |
| | | 3-yl | | | | | | | | | | | |
| 2245 | CH₂- | CH₂- | nBu | CH₂C | guani- | Me, | tBu | CH₂O | 14.6 | A | 1214.2 | 1.48 | 0.0278 |
| | pheny | indol- | | ONH₂ | dinyl | CH2O | | H | | | | | |
| | | 3-yl | | | | H | | | | | | | |
| 2246 | CH₂- | CH₂- | nBu | CH₂C | guani- | Me, | tBu | CH₂O | 10.3 | A | 1227 | 1.59 | 0.0470 |
| | pheny | indol- | | ONH₂ | dinyl | CHMe | | H | | | | | |
| | | 3-yl | | | | Et | | | | | | | |
| 2247 | CH₂- | CH₂- | nBu | CH₂C | guani- | Me, Me | tBu | CH₂O | 27.9 | B | 1206.3 | 1.88 | 0.0296 |
| | pheny | indol- | | ONH₂ | dinyl | | | H | | | | | |
| | | 3-yl | | | | | | | | | | | |
| 2248 | CH₂- | CH₂- | nBu | CH₂C | guani- | Me, | tBu | CH₂O | 25.3 | A | 1234.9 | 1.57 | 0.0454 |
| | pheny | indol- | | ONH₂ | dinyl | (CH2)2 | | H | | | | | |
| | | 3-yl | | | | COOH | | | | | | | |
| 2249 | CH₂- | CH₂- | nBu | CH₂C | guani- | F368 | tBu | CH₂O | 32.4 | A | 1258.9 | 1.62 | 0.0461 |
| | pheny | indol- | | ONH₂ | dinyl | | | H | | | | | |
| | | 3-yl | | | | | | | | | | | |
| 2250 | CH₂- | CH₂- | nBu | CH₂C | guani- | Me, | CMe | CH₂O | 24.9 | B | 1228 | 1.91 | 0.0504 |
| | pheny | indol- | | ONH₂ | dinyl | nBu | 2OH | H | | | | | |
| | | 3-yl | | | | | | | | | | | |
| 2251 | CH₂- | CH₂-4- | nBu | CH₂C | guani- | Me, | tBu | CH₂O | 27.4 | B | 1237.2 | 1.82 | 0.0286 |
| | phenyl | OH- | | ONH₂ | dinyl | nBu | | H | | | | | |
| | (4- | phenyl | | | | | | | | | | | |
| | COO | | | | | | | | | | | | |
| | H) | | | | | | | | | | | | |
| 2252 | CH₂- | CH₂-4- | nBu | CH₂C | guani- | Me, | tBu | CH₂O | 20.4 | A | 1223.1 | 1.64 | 0.0120 |
| | 4- | OH- | | ONH₂ | dinyl | nBu | | H | | | | | |
| | OH- | phenyl | | | | | | | | | | | |
| | phenyl | | | | | | | | | | | | |
| 2253 | CH₂- | CH₂- | nBu | CH₂C | guani- | Me, | tBu | CH₂O | 19.8 | A | 1227 | 1.59 | 0.0207 |
| | 4- | indol- | | ONH₂ | dinyl | nBu | | H | | | | | |
| | pyri- | 3-yl | | | | | | | | | | | |
| | dinyl | | | | | | | | | | | | |
| 2254 | CH₂- | CH₂- | nBu | CH₂C | guani- | H, | tBu | CH₂C | 45.5 | A | 1241.6 | 1.45 | 0.0279 |
| | pheny | indol- | | ONH₂ | dinyl | (CH2)4 | | OOH | | | | | |
| | | 3-yl | | | | NH2 | | | | | | | |
| 2255 | CH₂- | CH₂- | Nva | CH₂C | guani- | Me, | tBu | CH₂C | 36.1 | B | 1233.3 | 1.91 | 0.0268 |
| | pheny | indol- | | ONH₂ | dinyl | nBu | | OOH | | | | | |
| | | 3-yl | | | | | | | | | | | |
| 2256 | CH₂- | CH₂- | nBu | CH₂C | guani- | Me | tBu | CH₂O | 15.5 | B | 1248.3 | 1.88 | 0.0138 |
| | phenyl | indol- | | ONH₂ | dinyl | nBu | | H | | | | | |
| | (4- | 3-yl | | | | | | | | | | | |
| | COO | | | | | | | | | | | | |
| | H) | | | | | | | | | | | | |
| 2257 | CH₂- | CH₂-4- | nBu | CH₂C | guani- | Me, | tBu | CH₂O | 19.8 | A | 1215.5 | 1.42 | 0.0404 |
| | 4- | OH- | | ONH₂ | dinyl | nBu | | H | | | | | |
| | pyri- | phenyl | | | | | | | | | | | |
| | dinyl | | | | | | | | | | | | |
| 2258 | CH₂- | CH₂- | Nva | CH₂C | guani- | Me, | tBu | CH₂O | 26.8 | B | 1220 | 1.95 | 0.0369 |
| | pheny | indol- | | ONH₂ | dinyl | nBu | | H | | | | | |
| | | 3-yl | | | | | | | | | | | |
| 2259 | CH₂- | CH₂-4- | nBu | CH₂C | guani- | Me, | tBu | CH₂O | 35.5 | B | 1237 | 1.81, | 0.0381 |
| | phenyl | OH- | | ONH₂ | dinyl | nBu | | H | | | | 1.91 | |
| | (4- | phenyl | | | | | | | | | | | |
| | CON | | | | | | | | | | | | |
| | H₂) | | | | | | | | | | | | |
| 2260 | CH₂- | CH₂-4- | iBu | CH₂C | guani- | Me, | tBu | CH₂O | 9.3 | B | 1214.9 | 1.87 | 0.0160 |
| | pheny | OH- | | ONH₂ | dinyl | nBu | | H | | | | | |
| | | phenyl | | | | | | | | | | | |
| 2261 | CH₂- | CH₂-4- | iBu | CH₂C | guani- | Me, | tBu | CH₂C | 1.5 | A | 1229.2 | 1.51 | 0.0238 |
| | pheny | OH- | | ONH₂ | dinyl | nBu | | OOH | | | | | |
| | | phenyl | | | | | | | | | | | |
| 2262 | CH₂- | CH₂-4- | iBu | CH₂C | guani- | Me, | tBu | CH₂O | 13.6 | B | 1201.8 | 1.77 | 0.0398 |
| | pheny | OH- | | ONH₂ | dinyl | CH2OH | | H | | | | | |
| | | phenyl | | | | | | | | | | | |
| 2263 | CH₂- | CH₂- | iBu | CH₂C | guani- | Me, | tBu | CH₂O | 21.2 | B | 1227 | 1.94 | 0.0251 |
| | pheny | indol- | | ONH₂ | dinyl | iBu | | H | | | | | |
| | | 3-yl | | | | | | | | | | | |
| 2264 | CH₂- | CH₂- | iBu | CH₂C | guani- | Me, | tBu | CH₂C | 10 | A | 1228.1 | 1.47 | 0.0247 |
| | pheny | indol- | | ONH₂ | dinyl | CH2OH | | OOH | | | | | |
| | | 3-yl | | | | | | | | | | | |
| 2265 | CH₂- | CH₂- | iBu | CH₂C | guani- | Me, | tBu | CH₂C | 18.2 | A | 1241.1 | 1.53 | 0.0178 |
| | pheny | indol- | | ONH₂ | dinyl | nBu | | OOH | | | | | |
| | | 3-yl | | | | | | | | | | | |
| 2266 | CH₂- | CH₂- | iBu | CH₂C | guani- | Me, | tBu | CH₂O | 16.3 | B | 809.3 | 1.75 | 0.0379 |
| | pheny | indol- | | ONH₂ | dinyl | CH2OH | | H | | | | | |
| | | 3-yl | | | | | | | | | | | |
| 2267 | CH₂- | CH₂-4- | iBu | CH₂C | guani- | Me, | tBu | CH₂C | 8.7 | B | 1229.2 | 1.84 | 0.0289 |
| | pheny | OH- | | ONH₂ | dinyl | iBu | | OOH | | | | | |
| | | phenyl | | | | | | | | | | | |
| 2268 | CH₂- | CH₂- | iBu | CH₂C | guani- | Me, | tBu | CH₂O | 21 | B | 1227 | 1.95 | 0.0161 |
| | pheny | indol- | | ONH₂ | dinyl | nBu | | H | | | | | |
| | | 3-yl | | | | | | | | | | | |
| 2269 | CH₂- | CH₂- | iBu | CH₂C | guani- | Me, | tBu | CH₂C | 12.7 | B | 1241 | 1.92 | 0.0282 |
| | pheny | indol- | | ONH₂ | dinyl | iBu | | OOH | | | | | |
| | | 3-yl | | | | | | | | | | | |
| 2270 | CH₂- | CH₂-4- | iBu | CH₂C | guani- | Me, | tBu | CH₂O | 13 | A | 1215.2 | 1.55 | 0.0188 |
| | pheny | OH- | | ONH₂ | dinyl | iBu | | H | | | | | |
| | | phenyl | | | | | | | | | | | |
| 2271 | CH₂- | CH₂-4- | iBu | CH₂C | guani- | Me, iPr | tBu | CH₂O | 8.9 | A | 1207.9 | 1.48 | 0.0254 |
| | pheny | OH- | | ONH₂ | dinyl | | | H | | | | | |
| | | phenyl | | | | | | | | | | | |
| 2272 | CH₂- | CH₂- | iBu | CH₂C | guani- | Me, iPr | tBu | CH₂O | 5.3 | A | 1219.4 | 1.59 | 0.0344 |
| | pheny | indol- | | ONH₂ | dinyl | | | H | | | | | |
| | | 3-yl | | | | | | | | | | | |
| 2273 | CH₂- | CH₂- | iBu | CH₂C | guani- | Me, | tBu | CH₂C | 4.6 | A | 1234.3 | 1.49 | 0.0336 |
| | pheny | indol- | | ONH₂ | dinyl | CH(Me) | | OOH | | | | | |
| | | 3-yl | | | | OH | | | | | | | |
| 2274 | CH₂- | CH₂-4- | iBu | CH₂C | guani- | Me, iPr | tBu | CH₂C | 3.2 | B | 1222.1 | 1.71 | 0.0188 |
| | pheny | OH- | | ONH₂ | dinyl | | | OOH | | | | | |
| | | phenyl | | | | | | | | | | | |
| 2275 | CH₂- | CH₂-4- | iBu | CH₂C | guani- | Me, | tBu | CH₂C | 3 | B | 1223.2 | 1.68 | 0.0318 |
| | pheny | OH- | | ONH₂ | dinyl | CH(Me) | | OOH | | | | | |
| | | phenyl | | | | OH | | | | | | | |
| 2276 | CH₂- | CH₂- | iBu | CH₂C | guani- | Me, iPr | tBu | CH₂C | 4.2 | A | 8231 | 1.46 | 0.0300 |
| | pheny | indol- | | ONH₂ | dinyl | | | OOH | | | | | |
| | | 3-yl | | | | | | | | | | | |
| 2277 | CH₂- | CH₂-4- | iBu | CH₂C | guani- | Me, | tBu | iPr | 39 | A | 1221.1 | 1.6 | 0.0212 |
| | pheny | OH- | | ONH₂ | dinyl | nBu | | | | | | | |
| | | phenyl | | | | | | | | | | | |
| 2278 | CH₂- | CH₂-4- | iBu | CH₂C | NH2 | Me | tBu | CH₂O | 40.8 | B | 1194 | 1.86 | 0.0282 |
| | pheny | OH | | ONH₂ | | nBu | | H | | | | | |
| | | phenyl | | | | | | | | | | | |
| 2279 | CH₂- | CH₂-4- | iBu | CH₂C | NH2 | Me, | tBu | iPr | 46.8 | B | 1200 | 1.92 | 0.0286 |
| | pheny | OH- | | ONH₂ | | nBu | | | | | | | |
| | | phenyl | | | | | | | | | | | |
| 2280 | CH₂- | CH₂- | iBu | CH₂C | NH2 | Me, | tBu | CH₂O | 2.1 | A | 1205.8 | 1.79 | 0.0455 |
| | pheny | indol- | | ONH₂ | | nBu | | H | | | | | |
| | | 3-yl | | | | | | | | | | | |
| 2281 | CH₂- | CH₂- | iBu | CH₂C | guani- | Me. | tBu | iPr | 36.2 | B | 1232.2 | 2 | 0.0313 |
| | pheny | indol- | | ONH₂ | dinyl | nBu | | | | | | | |
| | | 3-yl | | | | | | | | | | | |
| 2282 | CH₂- | CH₂- | iBu | CH₂C | NH2 | Me, | tBu | iPr | 69.5 | B | 1211.3 | 2.01 | 0.0349 |
| | pheny | indol- | | ONH₂ | | nBu | | | | | | | |
| | | 3-yl | | | | | | | | | | | |
| 2283 | CH₂- | CH₂- | iBu | CH₂C | NH2 | Me, | tBu | CH₂C | 38.7 | B | 1219.1 | 1.9 | 0.0278 |
| | pheny | indol- | | ONH₂ | | nBu | | OOH | | | | | |
| | | 3-yl | | | | | | | | | | | |
| 2284 | CH₂- | CH₂-4- | iBu | CH₂C | Orn | Me, | tBu | CH₂C | 33.3 | A | 1208 | 1.46 | 0.0239 |
| | pheny | OH | | ONH | | nBu | | OOH | | | | | |
| | | phenyl | | | | | | | | | | | |

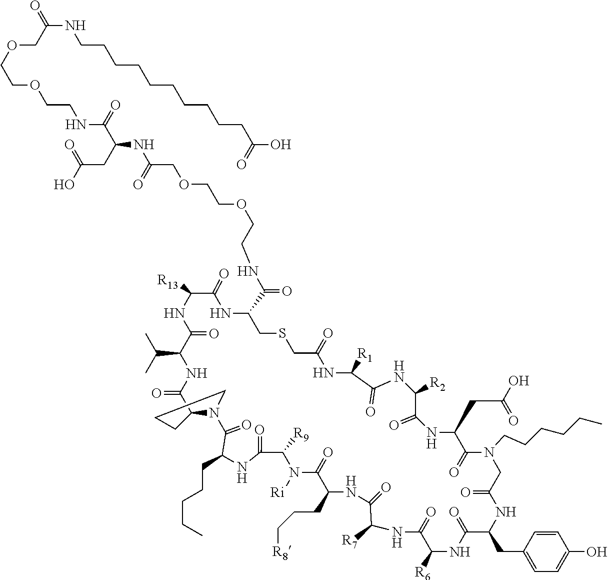

| | | | | | | | | | | | | HC |
|---|---|---|---|---|---|---|---|---|---|---|---|---|
| | | | | | | | | | | | | CBA |
| | | | | | | | | | QC | | | miPrI |
| | | | | | | | | | Meth- | Obs. | | STA |
| Ex. | | | | | | Rᵢ, | | Yield | od | MS | | IC50 |
| No.. | R₁ | R₂ | R₆ | R₇ | R_{8′} | R₉ | R₁₃ | (mg) | IDs | Ion | RT | (uM) |
| 2285 | CH₂- | CH₂- | iBu | CH₂CO | guani- | H, | CH₂OH | 9.9 | B | 1193.1 | 1.72 | 0.0152 |
| | phenyl | 4- | | NH₂ | dinyl | iBu | | | | | | |
| | | OH- | | | | | | | | | | |
| | | phenyl | | | | | | | | | | |
| 2286 | CH₂- | CH₂- | nBu | CH₂CO | guani- | H, | CH₂CO | 10.6 | A | 818 | 1.74 | 0.0253 |
| | phenyl | indol- | | NH₂ | dinyl | iBu | OH | | | | | |
| | (3- | 3-yl | | | | | | | | | | |
| | Me) | | | | | | | | | | | |
| 2287 | CH₂- | CH₂- | iPr | H | guani- | H, | CH₂CO | 13.1 | A | 799.2 | 1.69 | 0.0216 |
| | 2- | 4- | | | dinyl | iBu | OH | | | | | |
| | naph- | OH- | | | | | | | | | | |
| | thyl | phenyl | | | | | | | | | | |
| 2288 | CH₂- | CH₂- | nBu | CH₂CO | guani- | H, | CH₂OH | 5.7 | B | 1219 | 1.77 | 0.0086 |
| | 2- | 4- | | NH₂ | dinyl | iBu | | | | | | |
| | naph- | OH- | | | | | | | | | | |
| | thyl | phenyl | | | | | | | | | | |
| 2289 | CH₂- | CH₂- | iBu | CH₂CO | guani- | H, | iPr | 19.1 | B | 1199.2 | 1.98 | 0.0157 |
| | phenyl | phenyl | | NH₂ | dinyl | iBu | | | | | | |
| | | (3- | | | | | | | | | | |
| | | Me) | | | | | | | | | | |
| 2290 | CH₂- | CH₂- | nBu | CH₂CO | guani- | Me, | CH₂CO | 13.8 | A | 1251.2 | 1.51 | 0.0391 |
| | 2- | indol- | | NH₂ | dinyl | nBu | OH | | | | | |
| | naph- | 3-yl | | | | | | | | | | |
| | thyl | | | | | | | | | | | |
| 2291 | CH₂- | CH₂- | iBu | CH₂CO | guani- | H, | iPr | 19.3 | B | 1200.1 | 1.9 | 0.0174 |
| | phenyl | 4- | | NH₂ | dinyl | iBu | | | | | | |
| | | OH- | | | | | | | | | | |
| | | phenyl | | | | | | | | | | |
| 2292 | CH₂- | CH₂- | nPen- | H | guani- | H, | CH₂OH | 14.1 | B | 1172.3 | 1.79 | 0.0182 |
| | phenyl | 4- | tyl | | dinyl | iBu | | | | | | |
| | | OH- | | | | | | | | | | |
| | | phenyl | | | | | | | | | | |
| 2293 | CH₂- | CH₂- | nBu | CH₂CO | guani- | Me, | CH₂CO | 17.6 | A | 818 | 1.43 | 0.0257 |
| | phenyl | indol- | | NH₂ | dinyl | nBu | OH | | | | | |
| | | 3-yl | | | | | | | | | | |
| 2294 | CH₂- | CH₂- | nBu | H | guani- | H, | CH₂CO | 29.1 | B | 1186.9 | 1.74 | 0.0165 |
| | phenyl | 4- | | | dinyl | iBu | OH | | | | | |
| | (3- | OH- | | | | | | | | | | |
| | Me) | phenyl | | | | | | | | | | |
| 2295 | CH₂- | CH₂- | nBu | CH₂CO | guani- | H, | CH₂CO | 19 | B | 1208.4 | 1.72 | 0.0331 |
| | phenyl | 4- | | OH | dinyl | iBu | NH₂ | | | | | |
| | | OH- | | | | | | | | | | |
| | | phenyl | | | | | | | | | | |
| 2296 | CH₂- | CH₂- | Ahp | H | guani- | H, | CH₂OH | 4.6 | B | 787.2 | 1.84 | 0.0363 |
| | phenyl | 4- | | | dinyl | iBu | | | | | | |
| | | OH- | | | | | | | | | | |
| | | phenyl | | | | | | | | | | |
| 2297 | CH₂- | CH₂- | nBu | CH₂CO | guani- | H, | CH₂CO | 10.5 | B | 823 | 1.84 | 0.0211 |
| | phenyl | indol- | | NH₂ | dinyl | iBu | OH | | | | | |
| | (3- | 3-yl | | | | | | | | | | |
| | Me) | | | | | | | | | | | |
| 2298 | CH₂- | CH₂- | iPr | H | guani- | H, | CH₂CO | 4.8 | B | 1205 | 1.82, | 0.0397 |
| | 2- | 4- | | | dinyl | iBu | OH | | | | 1.86 | |
| | naph- | OH- | | | | | | | | | | |
| | thyl | phenyl | | | | | | | | | | |
| 2299 | CH₂- | CH₂- | nBu | CH₂CO | guani- | H, | CH₂CO | 5 | B | 1215 | 1.74 | 0.0209 |
| | phenyl | 4- | | OH | dinyl | iBu | NH₂ | | | | | |
| | | OH- | | | | | | | | | | |
| | | phenyl | | | | | | | | | | |
| 2300 | CH₂- | CH₂- | nBu | H | guani- | H, | CH₂CO | 5.4 | B | 1193.5 | 1.78 | 0.0351 |
| | phenyl | 4- | | | dinyl | iBu | OH | | | | | |
| | (3- | OH- | | | | | | | | | | |
| | Me) | phenyl | | | | | | | | | | |
| 2301 | CH₂- | CH₂- | nBu | CH₂CO | guani- | Me, | CH₂CO | 6.6 | B | 823.2 | 1.79 | 0.0199 |
| | phenyl | indol- | | NH₂ | dinyl | nBu | OH | | | | | |
| | | 3-yl | | | | | | | | | | |
| 2302 | CH₂- | CH₂- | iBu | CH₂CO | guani- | H, | iPr | 8.4 | A | 1206.8 | 1.65 | 0.0149 |
| | phenyl | 4- | | NH₂ | dinyl | iBu | | | | | | |
| | | OH- | | | | | | | | | | |
| | | phenyl | | | | | | | | | | |
| 2303 | CH₂- | CH₂- | nBu | CH₂CO | guani- | H, | CH₂OH | 9.5 | B | 1226.1 | 1.81 | 0.0099 |
| | 2- | 4- | | NH₂ | dinyl | iBu | | | | | | |
| | naph- | OH- | | | | | | | | | | |
| | thyl | phenyl | | | | | | | | | | |
| 2304 | CH₂- | F(3- | iBu | CH₂CO | guani- | H, | iPr | 11 | A | 1206 | 1.7 | 0.0262 |
| | phenyl | Me) | | NH₂ | dinyl | iBu | | | | | | |
| 2305 | CH₂- | CH₂- | nBu | CH₂CO | NHCO | H, | CH₂OH | 23.6 | B | 1219.3 | 1.84 | 0.0230 |
| | 2- | 4- | | NH₂ | NH₂ | iBu | | | | | | |
| | naph- | OH- | | | | | | | | | | |
| | thyl | phenyl | | | | | | | | | | |
| 2306 | CH₂- | F(3- | iBu | CH₂CO | NHCO | H, | iPr | 25.8 | B | 1199.9 | 2.1 | 0.0265 |
| | phenyl | Me) | | NH₂ | NH₂ | iBu | | | | | | |
| 2307 | CH₂- | CH₂- | nPen- | H | NHCO | H, | CH₂OH | 9.1 | A | 1173 | 1.63 | 0.0212 |
| | phenyl | 4- | tyl | | NH₂ | iBu | | | | | | |
| | | OH- | | | | | | | | | | |
| | | phenyl | | | | | | | | | | |
| 2308 | CH₂- | CH₂- | iPr | H | NHCO | H, | CH₂CO | 38.1 | B | 1198 | 1.75 | 0.0146 |
| | 2- | 4- | | | NH₂ | iBu | OH | | | | | |
| | naph- | OH- | | | | | | | | | | |
| | thyl | phenyl | | | | | | | | | | |
| 2309 | CH₂- | CH₂- | nBu | H | NHCO | H, | CH₂CO | 22.9 | B | 1187 | 1.79 | 0.0179 |
| | phenyl | 4- | | | NH₂ | iBu | OH | | | | | |
| | (3- | OH- | | | | | | | | | | |
| | Me) | phenyl | | | | | | | | | | |
| 2310 | CH₂- | CH₂- | nBu | CH₂CO | NHCO | Me, | CH₂OH | 5.6 | B | 1213 | 1.8 | 0.0245 |
| | phenyl | indol- | | NH₂ | NH₂ | iBu | | | | | | |
| | | 3-yl | | | | | | | | | | |
| 2311 | CH₂- | CH₂- | nBu | CH₂CO | NHCO | Tic | CH₂OH | 7.8 | B | 1229.8 | 1.6 | 0.0284 |
| | phenyl | indol- | | NH₂ | NH₂ | | | | | | | |
| | | 3-yl | | | | | | | | | | |
| 2312 | CH₂- | CH₂- | nBu | CH₂CO | guani- | Tic | CH₂OH | 28.1 | A | 1242.3 | 1.65 | 0.0315 |
| | 2- | 4- | | NH₂ | dinyl | | | | | | | |
| | naph- | OH- | | | | | | | | | | |
| | thyl | phenyl | | | | | | | | | | |
| 2313 | CH₂- | CH₂- | nBu | CH₂CO | guani- | Me, | CH₂OH | 23.7 | A | 1225.9 | 1.57 | 0.0281 |
| | 2- | 4- | | NH₂ | dinyl | iBu | | | | | | |
| | naph- | OH- | | | | | | | | | | |
| | thyl | phenyl | | | | | | | | | | |
| 2314 | CH₂- | CH₂- | nBu | CH₂CO | guani- | Me, | CH₂OH | 18.1 | B | 809 | 1.93 | 0.0346 |
| | phenyl | indol- | | NH₂ | dinyl | iBu | | | | | | |
| | | 3-yl | | | | | | | | | | |
| 2315 | CH₂- | CH₂- | nBu | CH₂CO | NHCO | Tic | CH₂OH | 11.9 | A | 828.6 | 1.63 | 0.0183 |
| | 2- | 4- | | NH₂ | NH₂ | | | | | | | |
| | naph- | OH- | | | | | | | | | | |
| | thyl | phenyl | | | | | | | | | | |
| 2316 | CH₂- | CH₂- | nBu | CH₂CO | NHCO | Me, | CH₂OH | 7.1 | B | 1226.9 | 1.94 | 0.0357 |
| | 2- | 4- | | NH₂ | NH₂ | iBu | | | | | | |
| | naph- | OH- | | | | | | | | | | |
| | thyl | phenyl | | | | | | | | | | |
| 2317 | CH₂- | CH₂- | nBu | CH₂CO | NHCO | Me, | CH₂OH | 6.4 | A | 1213 | 1.51 | 0.0183 |
| | phenyl | indol- | | NH₂ | NH₂ | nBu | | | | | | |
| | | 3-yl | | | | | | | | | | |
| 2318 | CH₂- | CH₂- | nBu | CH₂CO | guani- | Me, | CH₂OH | 12.7 | A | 1212.3 | 1.59 | 0.0213 |
| | phenyl | indol- | | NH₂ | dinyl | nBu | | | | | | |
| | | 3-yl | | | | | | | | | | |
| 2319 | CH₂- | CH₂- | nBu | CH₂CO | NHCO | Me, | CH₂OH | 8.6 | B | 817.94, | 1.77, | 0.0251 |
| | 2- | 4- | | NH₂ | NH₂ | nBu | | | | 817.94 | 1.79 | |
| | naph- | OH- | | | | | | | | | | |
| | thyl | phenyl | | | | | | | | | | |
| 2320 | CH₂- | CH₂- | nBu | CH₂CO | guani- | Me, | CH₂OH | 10.1 | A | 1226.2 | 1.67 | 0.0226 |
| | 2- | 4- | | NH₂ | dinyl | nBu | | | | | | |
| | naph- | OH- | | | | | | | | | | |
| | thyl | phenyl | | | | | | | | | | |

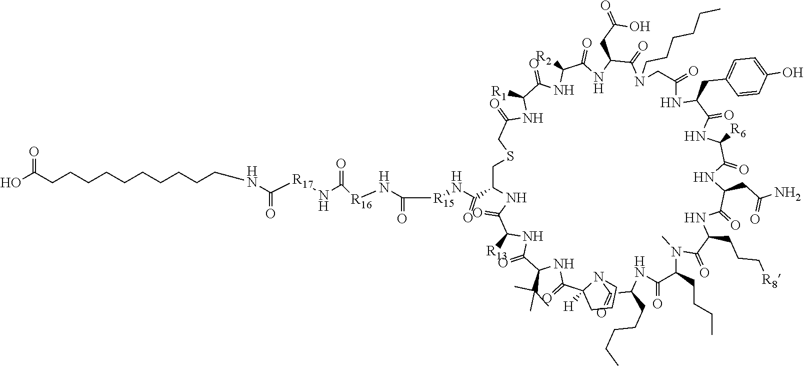

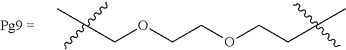 [SMILES: *CCOCCOC*]

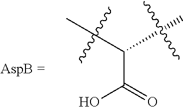

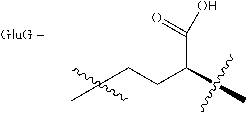

| | | | | | | | | | | | | | HC |
|---|---|---|---|---|---|---|---|---|---|---|---|---|---|
| | | | | | | | | | | QC | | | CBA |
| | | | | | | | | | | Meth- | Obs. | | mVISTA |
| Ex. | | | | | | | | | Yield | od | MS | | IC50 |
| No. | R₁ | R₂ | R₆ | R_{8′} | R₁₃ | R₁₅ | R₁₆ | R₁₇ | (mg) | IDs | Ion | RT | (uM) |
| 23 | CH₂- | CH₂-4- | iBu | guani- | CH₂CO | AspB | Pg9 | Asn | 23.9 | B | 1265.1 | 1.73 | 0.0155 |
| 21 | phenyl | OH- | | dinyl | OH | | | B | | | | | |
| | | phenyl | | | | | | | | | | | |
| 23 | CH₂- | CH₂-4- | iBu | guani- | CH₂OH | AspB | Pg9 | Asn | 8.8 | B | 1251.2 | 1.6 | 0.0273 |
| 22 | phenyl | OH- | | dinyl | | | | B | | | | | |
| | | phenyl | | | | | | | | | | | |
| 23 | CH₂- | CH₂-4- | iBu | guani- | CH₂OH | GluG | Dab | | 20.4 | A | 1119.9 | 1.61 | 0.0339 |
| 23 | phenyl | OH- | | dinyl | | | | | | | | | |
| | | phenyl | | | | | | | | | | | |
| 23 | CH₂- | CH₂-4- | iBu | NH₂ | CH₂OH | GluG | Dab | | 90.6 | A | 1099.1 | 1.58 | 0.0221 |
| 24 | phenyl | OH- | | | | | | | | | | | |
| | | phenyl | | | | | | | | | | | |
| 23 | CH₂- | CH₂- | nBu | guani- | CH₂OH | CH₂ | AspB | Pg9 | 32.2 | B | | 1.94 | 0.0192 |
| 25 | phenyl | indol- | | dinyl | | | | | | | | | |
| | | 3-yl | | | | | | | | | | | |
| 23 | CH₂- | CH₂-4- | nBu | guani- | CH₂OH | CH₂ | AspB | Pg9 | 39.5 | A | 1164 | 1.58 | 0.0167 |
| 26 | phenyl | OH- | | dinyl | | | | | | | | | |
| | | phenyl | | | | | | | | | | | |
| 23 | CH₂- | CH₂- | nBu | guani- | CH₂OH | CH₂ | AspB | Pg9 | 24.9 | B | 776.2 | 1.91 | 0.0337 |
| 27 | phenyl | phenyl | | dinyl | | | | | | | | | |
| | | (3-Me) | | | | | | | | | | | |
| 23 | CH₂- | CH₂- | nBu | guani- | CH₂OH | CH₂ | AspB | Pg9 | 9.3 | A | 786.6 | 1.67 | 0.0405 |
| 28 | phenyl | indol- | | dinyl | | | | | | | | | |
| | (3-Me) | 3-yl | | | | | | | | | | | |

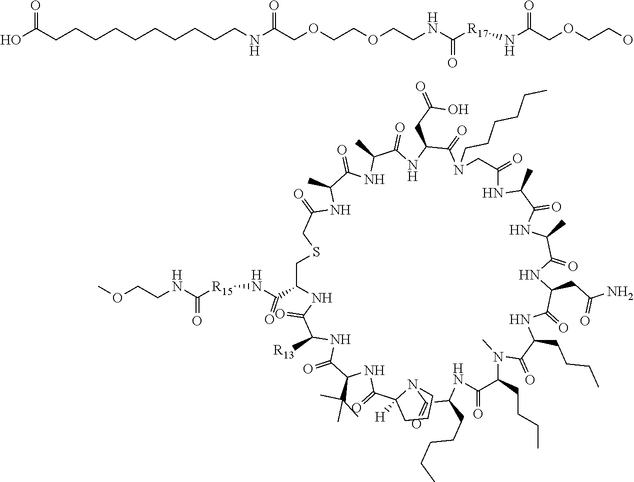

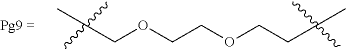 [SMILES: *CCOCCOC*]

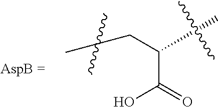

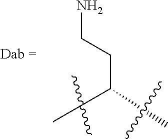

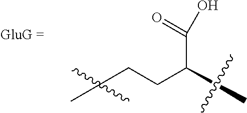

| | | | | | | | | | | | | | HC |
|---|---|---|---|---|---|---|---|---|---|---|---|---|---|
| | | | | | | | | | | | | | CBA |
| | | | | | | | | | | QC | | | mVIS |
| Ex. | | | | | | | | | | Meth- | Obs. | | TA |
| Num- | | | | | | | | | Yield | od | MS | | IC50 |
| ber | R₁ | R₂ | R₅ | R₆ | R_{8′} | R₁₃ | R₁₅ | R₁₇ | (mg) | IDs | Ion | RT | (uM) |
| 2329 | CH₂- | CH₂- | CH₂- | iBu | guani- | CH₂CO | AspB | Dab | 27 | B | 1272.1 | 1.68 | 0.0224 |
| | phenyl | 4-OH- | 4- | | dinyl | OH | | | | | | | |
| | | phenyl | OH- | | | | | | | | | | |
| | | | phenyl | | | | | | | | | | |
| 2330 | CH₂- | CH₂- | CH₂- | nBu | guani- | CH₂OH | CH₂ | GluG | 55.7 | A | 829 | 1.72 | 0.0180 |
| | phenyl | Phenyl | 4- | | dinyl | | | | | | | | |
| | | (3- | OH- | | | | | | | | | | |
| | | Me) | phenyl | | | | | | | | | | |
| 2331 | CH₂- | CH₂- | CH₂- | nBu | guani- | CH₂OH | CH₂ | GluG | 49.6 | A | 1243.9 | 1.57 | 0.0137 |
| | phenyl | 4-OH- | 4- | | dinyl | | | | | | | | |
| | | phenyl | OH- | | | | | | | | | | |
| | | | phenyl | | | | | | | | | | |
| 2332 | CH₂- | CH₂- | CH₂- | nBu | guani- | CH₂OH | CH₂ | GluG | 31.4 | B | 1255 | 1.91 | 0.0223 |
| | phenyl | indol- | 4- | | dinyl | | | | | | | | |
| | | 3-yl | OH- | | | | | | | | | | |
| | | | phenyl | | | | | | | | | | |
| 2333 | CH₂- | CH₂- | CH₂- | nBu | guani- | CH₂OH | CH₂ | GluG | 41.1 | A | 1269.4 | 1.55 | 0.0468 |
| | 2- | 4-OH- | 4- | | dinyl | | | | | | | | |
| | naph- | phenyl | OH- | | | | | | | | | | |
| | thyl | | phenyl | | | | | | | | | | |
| 2334 | CH₂- | CH₂- | CH₂- | iBu | guani- | CH₂OH | AspB | Orn | 26.6 | B | 1265 | 1.7 | 0.0330 |
| | Phenyl | 4-OH- | 4- | | dinyl | | | | | | | | |
| | | phenyl | OH- | | | | | | | | | | |
| | | | phenyl | | | | | | | | | | |
| 2335 | CH₂- | CH₂- | CH₂- | iBu | guani- | CH₂CO | AspB | Orn | 24.2 | A | 1278.9 | 1.44 | 0.0167 |
| | phenyl | 4-OH- | 4- | | dinyl | OH | | | | | | | |
| | | phenyl | OH- | | | | | | | | | | |
| | | | phenyl | | | | | | | | | | |
| 2336 | CH₂- | CH₂- | CH₂- | iBu | NH₂ | CH₂OH | AspB | Asp | 5.7 | A | 1262.1 | 1.56 | 0.0240 |
| | phenyl | 4-OH- | 4- | | | | | | | | | | |
| | | phenyl | OH- | | | | | | | | | | |
| | | | 3- | | | | | | | | | | |
| | | | Cl- | | | | | | | | | | |
| | | | phenyl | | | | | | | | | | |

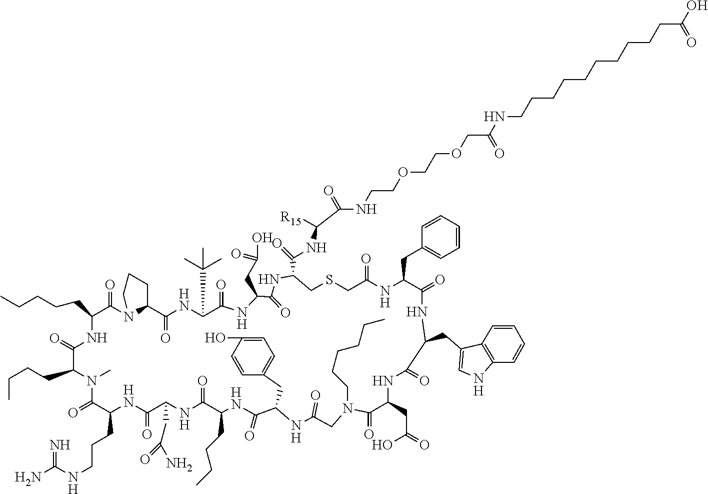

| | | | | | | HC CBA |
|---|---|---|---|---|---|---|
| | | | QC | | | mVISTA |
| | | Yield | Method | Obs. | | IC50 |
| Example Number | R₁₅ | (mg) | IDs | MS Ion | RT | (uM) |
| 2337 | CH2OH | 10.6 | A | 1147.1 | 1.63 | 0.0217 |
| 2338 | CH₂CONH₂ | 42.9 | A | 1161 | 1.65 | 0.0520 |
| 2339 | CH2NH2 | 10.7 | B | 1147.1 | 1.88 | 0.0261 |

### Example 2341

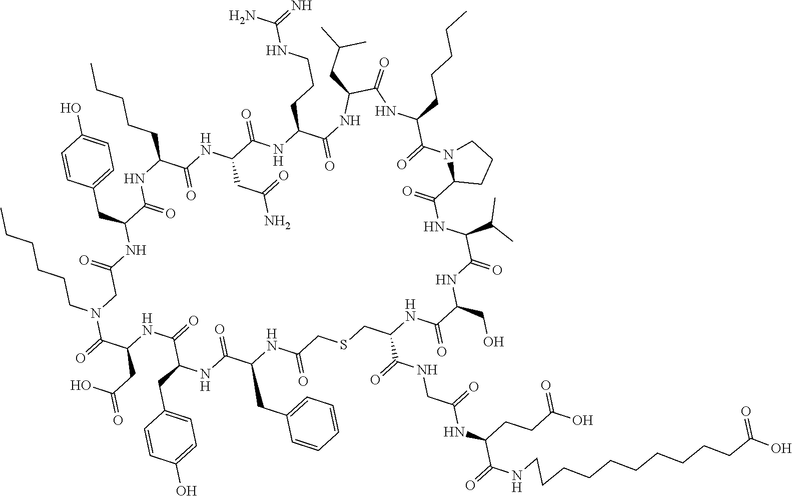 [SMILES: CCCCCCN1CC(=O)N[C@@H](Cc2ccc(O)cc2)C(=O)N[C@@H](CCCCC)C(=O)N[C@@H](CC(N)=O)C(=O)N[C@@H](CCCNC(=N)N)C(=O)N[C@@H](CC(C)C)C(=O)N[C@@H](CCCCC)C(=O)N2CCC[C@H]2C(=O)N[C@@H](C(C)C)C(=O)N[C@@H](CO)C(=O)N[C@H](C(=O)NCC(=O)N[C@@H](CCC(=O)O)C(=O)NCCCCCCCCCCC(=O)O)CSCC(=O)N[C@@H](Cc2ccccc2)C(=O)N[C@@H](Cc2ccc(O)cc2)C(=O)N[C@@H](CC(=O)O)C1=O]

| | | | Obs. | | |
|---|---|---|---|---|---|
| Example | Yield | QC Method | MS | | |
| No. | (mg) | IDs | Ion | RT | HC CBA mVISTA IC50 (uM) |
| 2341 | 14.8 | A | 1091.2 | 1.98 | 0.058 |

We claim:

1. A compound of Formula (I): 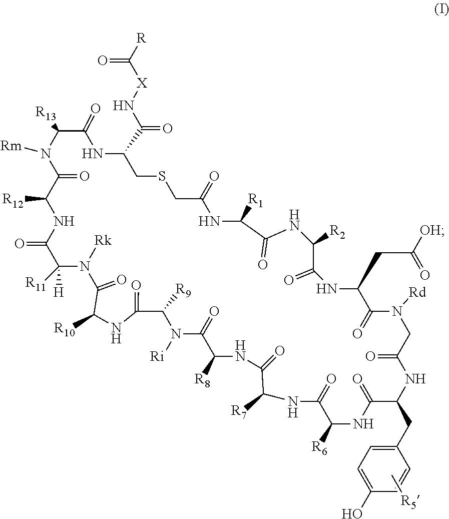
or a pharmaceutically acceptable salt thereof, wherein
R₁ is selected from the group consisting of arylC₁-C₃alkyl, and heteroarylC₁-C₃alkyl; wherein the aryl part of the arylC₁-C₃alkyl is optionally substituted with one, two, or three groups independently selected from halo, nitro, amino, C₁-C₄alkyl, aminocarbonyl, hydroxy, aminoC₁-C₄alkyl, aminoC₂-C₆alkoxy, trifluoromethyl, oxotrifluoromethyl, carboxy, cyano, carboxyC₁-C₄alkyl and carboxyC₁-C₄alkoxy; and
the heteroaryl part of the heteroarylC₁-C₃alkyl is optionally substituted with one, two, or three groups independently selected from C₁-C₃alkyl or halo;
R₂ is selected from the group consisting of arylC₁-C₃alkyl, and heteroarylC₁-C₃alkyl; wherein the aryl part of the arylC₁-C₃alkyl is optionally substituted with one, two, or three groups independently selected from halo, C₁-C₄alkyl, hydroxy, trifluoromethyl, oxotrifluoromethyl, and cyano; and
the heteroaryl part of the heteroarylC₁-C₃alkyl is optionally substituted with one, two, or three groups independently selected from C₁-C₃alkyl or halo;
R₅′ is selected from hydrogen or halo;
R₆ is selected from the group consisting of C₁-C₆alkyl, C₃-C₆cycloalkyl, aminoC₃-C₆alkyl, carboxyC₃-C₆alkyl, guanidinylC₃-C₆alkyl, and arylC₁-C₆alkyl; wherein the aryl part of the arylC₁-C₆alkyl is optionally substituted with halo, nitro, hydroxy, carboxy, and carboxyC₁-C₃alkoxy;
R₇ is selected from the group consisting of hydrogen, C₁-C₆alkyl, carboxyC₁-C₄alkyl, aminoC₁-C₄alkyl, and aminocarbonylC₁-C₄alkyl;
R₈ is selected from the group consisting of C₁-C₆alkyl, guanidinylC₃-C₆alkyl, aminoC₁-C₆alkyl, and aminocarbonylaminoC₃-C₆alkyl;
R₉ is selected from the group consisting of hydrogen, C₁-C₆alkyl, guanidinylC₁-C₄alkyl, carboxyC₁-C₄alkyl, hydroxyC₁-C₄alkyl, aminocarbonylC₁-C₄alkyl, aminoC₁-C₄alkyl, arylC₁-C₆alkyl, and heteroarylC₁-C₆alkyl; wherein the aryl part of the arylC₁-C₆alkyl is optionally substituted with halo or hydroxy;
R₁₀ is selected from the group consisting of C₃-C₆alkyl, C₃-C₆cycloalkyl, and phenylC₂-C₄alkyl;
R₁₁ is C₁-C₄alkyl;
R₁₂ is selected from the group consisting of C₃-C₆alkyl, and C₃-C₆cycloalkyl;
R₁₃ is selected from the group consisting of hydrogen, C₁-C₆alkyl, C₃-C₆cycloalkyl, carboxyC₁-C₄alkyl, hydroxyC₁-C₄alkyl, guanidinylC₃-C₆alkyl, aminocarbonylC₁-C₄alkyl; arylC₁-C₃alkyl, and heteroarylC₁-C₃alkyl;
R_{d} is C₄₋₆alkyl or C₂-C₄-aryl;
Rᵢ is hydrogen or C₁-C₆alkyl; or
Rᵢ and R₉, together with the carbon atom to which they are attached, form a 5-6 ring heterocycle ring, wherein the heterocycle is optionally fused with an aryl ring;
Rₖ is methyl; or
Rₖ and R₁₁, together with the carbon atom to which they are attached, form a 4-6 ring heterocycle ring, wherein the heterocycle is optionally substituted with halo, hydroxy or phenyl group;
Rₘ is hydrogen or methyl; or
Rₘ and R₁₃, together with the carbon atom to which they are attached, form a 5-6 ring heterocycle ring, wherein the heterocycle is optionally substituted with a hydroxy group;
R is NH₂, OH or NH(CH₂)₁₀₋₁₂COOH; and
X is selected from the group consisting of —X₁—, X₁—CONH—X₂—, X₁—CONH—X₂—CONH—X₃—, X₁—CONH—X₂—CONH—X₃—CONH—X₄—, wherein X₁, X₂, X₃, and X₄ are independently selected from CH₂, or any natural or unnatural amino acid side chains, or —(CH₂CH₂O)₂₋₃—; or
X together with COR is a hydrogen.

2. The compound according to claim 1 wherein
R₁ is selected from the group consisting of benzyl, naphthyl, or heteroaryl-CH₂; wherein the aryl part of the benzyl group is optionally substituted with one, two, or three groups independently selected from fluoro, chloro, bromo, nitro, amino, C₁-C₃alkyl, aminocarbonyl, hydroxy, aminoC₁-C₄alkyl, aminoC₂-C₆alkoxy, trifluoromethyl, oxotrifluoromethyl, carboxy, cyano, carboxyC₁-C₂alkyl, and carboxymethoxy.

3. The compound according to claim 1 wherein
R₂ is selected from the group consisting of benzyl, naphthyl, or heteroaryl-CH₂; wherein the aryl part of the benzyl group is optionally substituted with one or two groups independently selected from fluoro, chloro, C₁-C₃alkyl, hydroxy, trifluoromethyl, and cyano; and
the heteroaryl part of the heteroarylC₁-C₃alkyl is optionally substituted with one, two, or three groups independently selected from C₁-C₃alkyl.

4. The compound according to claim 1 wherein R₅′ is selected from hydrogen or chloro.

5. The compound according to claim 1 wherein
R₆ is selected from the group consisting of C₂-C₅alkyl, C₃-C₆cycloalkyl, aminoC₃-C₅alkyl, carboxyC₃-C₅alkyl, guanidinylC₃-C₅alkyl, and benzyl; wherein the aryl part of the benzyl group is optionally substituted with fluoro, chloro, nitro, hydroxy, carboxy, and carboxyC₁-C₂alkoxy.

6. The compound according to claim 1 wherein
R₇ is selected from hydrogen, C₁-C₄alkyl, carboxyC₁-C₂alkyl, aminoC₁-C₄alkyl, and aminocarbonylC₁-C₂alkyl.

7. The compound according to claim 1 wherein
R₈ is selected from the group consisting of C₁-C₄alkyl, guanidinylC₃-C₄alkyl, aminoC₁-C₄alkyl, and aminocarbonylaminoC₃-C₄alkyl.

8. The compound according to claim 1 wherein
R₉ is selected from the group consisting of hydrogen, C₁-C₄alkyl, guanidinylC₃-C₄alkyl, carboxyC₁-C₂alkyl, hydroxyC₁-C₄alkyl, aminocarbonylC₁-C₃alkyl, aminoC₁-C₄alkyl, benzyl, and heteroaryl-CH₂; wherein the aryl part of the benzyl group is optionally substituted with hydroxy; or
Rᵢ and R₉, together with the carbon atom to which they are attached, form a 5-6 ring heterocycle ring, wherein the heterocycle is optionally fused with phenyl ring.

9. The compound according to claim 1 wherein R₁₀ is selected from the group consisting of C₄-C₆alkyl, C₃-C₆cycloalkyl, and phenylC₂-C₄alkyl.

10. The compound according to claim 1 wherein when Rₖ is methyl, R₁₁ is C₁-C₄alkyl; alternatively, Rₖ and R₁, together with the carbon atom to which they are attached, form a pyrrolidinyl, azetidinyl, morpholinyl, or piperidinyl ring, wherein the heterocycle is optionally substituted with fluoro, hydroxy or phenyl group.

11. The compound according to claim 1 wherein
R₁₂ is selected from the group consisting of C₃-C₄alkyl, and C₃-C₅cycloalkyl.

12. The compound according to claim 1 wherein R₁₃ is selected from the group consisting of hydrogen, C₁-C₄alkyl, C₃-C₅cycloalkyl, carboxyC₁-C₂alkyl, hydroxyC₁-C₃alkyl, guanidinylC₃-C₄alkyl, aminocarbonylC₁-C₄alkyl; benzyl, and heteroaryl-CH₂; or
Rₘ and R₁₃, together with the carbon atom to which they are attached, form a pyrrolidinyl or piperidinyl ring, wherein the heterocycle is optionally substituted with a hydroxy group.

13. The compound according to claim 1 wherein R is selected from the group consisting of NH₂, OH, NH(CH₂)₁₀COOH, or NH(CH₂)₁₂COOH.

14. The compound according to claim 1 wherein X₁, X₂, X₃, or X₄ are selected from the group consisting of CH₂, CH(CH₂COOH), CH(CH₂OH), CH(CH₂CH₂COOH), CH(CH₂NH₂), CH(CH₂CH₂NH₂), CH(CH₂CH₂CH₂NH₂), CH(CH₂CH₂CH₂CH₂NH₂), CH(CH₂CONH₂), CH(CH₂CH₂CONH₂), CH(CH₂propargyl), CH(CH₂CH₂CH₂guanidinyl), CH(CH₂(4-hydroxyphenyl)), CH(CH₂indol-3-yl), (CH₂CH₂O)₂, CH(CH₂CH₂)(CH₂CH₂), CH(COOH)CH₂, and CH₂CH(COOH).

15. The compound according to claim 1 or the pharmaceutically acceptable salt thereof, wherein
R₁ is benzyl, 2-pyridinylmethyl, 1-napthylmethyl, 2-naphthylmethyl, 4-indolylmethyl, 3-indolylmethyl, or 3-benzothiophenemethyl, 2-methylphenylmethyl, 2-O-allyl-phenylmethyl, 3,4,5-trifluorophenylmethyl, 3,4-dimethoxyphenylmethyl, 3-trifluoromethylphenylmethyl, 3-chlorophenylmethyl, 3-methylphenylmethyl, 3-bromophenylmethyl, 4-trifluoromethylphenylmethyl, 4-methylphenylmethyl, 4-fluorophenylmethyl, 4-iodophenylmethyl, 4-cyanophenylmethyl, 4-aminocarbonylphenylmethyl, 4-aminophenylmethyl, 4-hydroxyphenylmethyl, 4-ethoxyphenylmethyl, 4-O-allylphenylmethyl, 4-methoxyphenylmethyl, and 2,4-difluorophenylmethyl;
R₂ is benzyl, 2-cyanophenylmethyl, 2-O-allyl-phenylmethyl, 3-chlorophenylmethyl, 3-bromophenylmethyl, 3-methylphenylmethyl, 3-cyanophenylmethyl, 3-fluorophenylmethyl, 4-methylphenylmethyl, 4-trifluoromethylphenylmethyl, 4-hydorxyphenylmethyl, 3-indolylmethyl, N-methyl-3-indolylmethyl, and 2,4-difluorophenylmethyl;
R₅′ is hydrogen or chloro;
R₆ is selected from the group consisting of methyl, ethyl, CHMeEt, n-pentyl, isopropyl, n-propyl, isobutyl, n-butyl, cyclopropyl, cyclohexyl, 3-carboxyphenylmethyl, 4-carboxyphenylmethyl, 4-COOH—CH₂O-phenylmethyl, aminobutyl, carboxypropyl, and guanidinylpropyl;
R₇ is selected from the group consisting of hydrogen, methyl, carboxymethyl, carboxyethyl, aminobutyl, and aminocarbonylmethyl;
R₈ is selected from the group consisting of methyl, guanidinylpropyl, aminoethyl, aminopropyl, aminobutyl, and aminocarbonylaminopropyl;
R₉ is selected from the group consisting of hydrogen, methyl, isopropyl, CHMeEt, n-butyl, isobutyl, guanidinylpropyl, carboxymethyl, carboxyethyl, hydroxymethyl, hydroxyCHMe, aminocarbonylmethyl, aminobutyl, 4-carboxyphenylmethyl, 3-carboxyphenylmethyl, 4-hydroxyphenylmethyl, 3-indolylmethyl, 4-COOH—CH₂—O-phenylmethyl; or,
when Ri is n-hexyl, R₉ is hydrogen; or,
Rᵢ and R₉, together with the carbon atom to which they are attached, form tetrahydroisoquinolin-3-yl;
R₁₀ is selected from the group consisting of npentyl, cyclopentyl, cyclopropyl, and phenylpropyl;
when Rₖ is methyl, R₁₁ is methyl, n-butyl, or isobutyl, or,
Rₖ and R₁₁, together with the carbon atom to which they are attached, form pyrrolidinyl, fluoropyrrolidinyl, hydroxypyrrolidinyl, phenylpyrrolidinyl, azetidinyl, morpholinyl, or piperidinyl ring;
R₁₂ is selected from the group consisting of tert-butyl, isopropyl, C(OH)(CH₃)₃, CH(CH₃)(CH₂CH₃), CH(CH₂CH₃)₂, cyclopropyl, and benzyl;
R₁₃ is selected from the group consisting of hydrogen, methyl, cyclopropyl, npentyl, isopropyl, carboxymethyl, carboxyethyl, hydroxymethyl, OH—CH(CH₃), isobutyl, guanidinylpropyl, aminocarbonylmethyl; benzyl, 4-hydroxyphenylmethyl, and 3-indolylmethyl; and Rₘ is hydrogen or methyl; or
Rₘ and R₁₃, together with the carbon atom to which they are attached, form a pyrrolidinyl or piperidinyl, or hydroxypyrrolidinyl ring.

16. A pharmaceutical composition comprising a compound according to claim 1 and a pharmaceutically acceptable carrier therefor.
